(19) United States
(12) Patent Application Publication
(10) Pub. No.: US 2008/0227813 A1
(43) Pub. Date: Sep. 18, 2008

(54) Pharmaceutical compositions and methods for treating diseases associated with neurodegeneration

(76) Inventor: Jack Raymond Barber, San Diego, CA (US)

Correspondence Address: ROPES & GRAY LLP, PATENT DOCKETING 39/361, 1211 AVENUE OF THE AMERICAS, NEW YORK, NY, 10036-8704, US

(21) Appl. No.: 11/904,534

(22) Filed: Sep. 26, 2007

Related U.S. Application Data
(60) Provisional application No. 60/847,606, filed on Sep. 26, 2006.
(60) Provisional application No. 60/852,791, filed on Oct. 18, 2006.

(51) Int. Cl. A61K 31/4545; A61K 31/15; A61P 25/00

(52) U.S. Cl. 514/318; 514/640

(57) ABSTRACT

The present invention relates to methods for treating diseases, conditions or disorders using hydroxyamine compounds, and in particular, N-[2-hydroxy-3-(1-piperidinyl)-propoxy]-pyridine-1-oxide-3-carboximidoyl chloride (Compound I) alone or in combination with one or more other therapeutic agents for the treatment of conditions, disorders or diseases associated with neurodegeneration in the central nervous system. Additional therapeutic agents are provided. The present invention also relates to pharmaceutical compositions comprising hydroxyamine compounds, an additional therapeutic agent and a pharmaceutically acceptable carrier and methods for treating diseases using them.

This application claims the benefit of U.S. Provisional Application Nos. 60/847,606, filed Sep. 26, 2006, and 60/852,791 filed Oct. 18, 2006, each of which is incorporated by reference herein in its entirety.

### FIELD OF THE INVENTION

The present invention relates to methods for treating conditions, disorders or diseases, using hydroxyamine compounds, in particular, N-[2-hydroxy-3-(1-piperidinyl)-propoxy]-pyridine-1-oxide-3-carboximidoyl chloride (Compound I) alone or in combination with at least one additional therapeutic agent useful for treating conditions, diseases or disorders associated with neurodegeneration in the central nervous system. The present invention also relates to pharmaceutical compositions comprising other hydroxyamine compounds, alone or in combination with an additional therapeutic agent, and a pharmaceutically acceptable carrier and methods for treating conditions, disorders or diseases using them, especially those associated with neurodegeneration in the central nervous system.

### BACKGROUND OF THE INVENTION

Molecular chaperones play an essential role in a variety of cellular processes. For example, molecular chaperones bind noncovalently to nascent proteins and partially folded intermediates, and guide them along correct protein folding pathways, thereby preventing their irreversible aggregation and misfolding. Molecular chaperones also unfold proteins for their translocation across intracellular membranes into organelles. In addition, molecular chaperones facilitate the degradation of misfolded proteins.

Many molecular chaperones are heat shock proteins (HSP), that is, proteins whose expression is increased when the cell is exposed to elevated temperatures or other cellular stresses. HSPs are also referred to as “stress proteins” and their upregulation is sometimes described more generally as part of the “stress response”. The increase in HSP expression induced by a cellular stress appears to protect the cell against what would otherwise be a lethal exposure. Cellular stresses that induce HSP expression include a wide variety of pathological conditions that are associated with many disorders and disease states.

One such pathological condition is ischemic injury. An ischemic injury to a tissue is caused by a decrease in the blood supply to the tissue. For instance, prolonged coronary occlusion causes severe damage to the myocardium, leading to myocardial necrosis and jeopardizing the chances for recovery, even if the blood flow is restored. In the brain, significant damage may frequently be caused by ischemia, leading to the death of brain tissue.

During an ischemic injury to the myocardium, it has been observed that the amount of a particular HSP, hsp70, increases, even if the duration of ischemia is short. In such a case, the enhanced hsp70 expression protects the cell against the consequences of another ischemic injury. See, e.g., DAS, D. K. et al. Cardiovascular Res.: 578. 1993. This has also been observed when rat cells in culture were subjected to ischemia injury. See, J. Clin. Invest., 93: 759-767 (1994).

Likewise, ischemic injury in the brain leads to an increase in HSP expression in the brain tissue. It has been shown that pretreatment of animals with sub-lethal ischemia induces hsp70 expression and protects the brain against more severe subsequent ischemic insult. See, Simon et al., Neurosci. Lett., 163:135-137 (1993).

Another pathological condition associated with molecular chaperone expression is free radical injury. Free radicals are highly unstable molecules produced by cells during normal metabolism, the major source being the mitrochondrion. If free radicals are not neutralized, they can accumulate and cause random damage to DNA, membrane lipids and proteins within the cell. With age, the balance between the production of a free radical reactive oxygen species (ROS) and its neutralization becomes impaired. This imbalance has been implicated in many neurodegenerative diseases of the central nervous system, including Alzheimer's disease (AD), Parkinson's disease (PD), multiple sclerosis (MS), neuropathies, Huntington's disease (HD) and amyotrophic lateral sclerosis (ALS). See, e.g., Lev N et al.: Apoptosis and Parkinson's disease; Progress in Neuro-Psychopharmacology and Biological Psychiatry 27: 245-50, 2003.

AD is the most common neurodegenerative disease and the most common form of dementia (responsible for about 80% of all cases). It is characterized by memory loss, language deterioration, impaired visuospatial skills, poor judgment, indifferent attitude, but preserved motor function. Symptoms of AD first appear as memory decline and, over several years, cognition, personality, and the ability to function is lost.

While the exact initiating events leading to AD are complex, it is widely accepted that neuronal death is mediated partly by free radical injury. See, Pratico D and Delanty N: Oxidative injury in diseases of the central nervous system: Focus on Alzheimer's disease, Am J Med 109: 577-85, 2000. The results are amyloid plaques and neurofibrillary tangles in the brain, as well as a loss of nerve cells in areas of the brain that are vital to memory and other mental abilities.

To date, there is no cure today for AD and patients usually live about 8 to 10 years from the time of diagnosis. Presently, the symptoms of AD are treated by cholinesterase inhibitors, such as Exelon, Reminyle and Aricept. See, Neurodegenerative Disorders: The world market 2002-207; a Visiongain Report; VISIONGAIN™, 2003; see also: Terry A V and Buccafusco J J: The cholinergic hypothesis of age and Alzheimer's disease related cognitive deficits: recent challenges and their implications for novel drug development; The Journal of pharmacology and experimental therapeutics, 306: 821-27, 2003; and Cummings J L: Use of cholinesterase inhibitors in clinical practice: evidence based recommendations; Am J Geriatr Psychiatry 11: 131-45, 2003.). Other treatments for AD include the antioxidant Ginkobiloba extract, nonsteroidal anti-inflammatory agents, and non-specific NMDA antagonists, such as Ebixa (Memantine). Another approach to treating AD is to develop drugs that decrease amyloid beta production and clearing of amyloid deposits through immunization.

The second most common neurodegenerative disease is PD. PD is characterized by tremors, or the involuntary and rhythmic movements of the hands, arms, legs and jaw. Classically, tremors appear while the individual is at rest and improve with intentional movement. Over time, there is a gradual loss of spontaneous movement, which often leads to a variety of other problems, such as “freezing”, decreased mental skill or quickness, voice changes, and decrease facial expression. In addition, muscles become rigid and the limbs become stiff. This results in postural instability, or a stooped, flexed posture with bending at the elbows, knees and hips. Further, there is a gradual loss of automatic movement, including eye blinking and decreased frequency of swallowing. Walking becomes unsteady. PD patients may also suffer from depression and dementia.

PD occurs when certain brain cells in an area of the brain, known as the substantia nigra, die or become impaired. These neurons produce an important chemical known as dopamine, a chemical messenger responsible for transmitting signals between the substantia nigra and the corpus striatum. The exact cause of neuronal death is unknown, but studies have implicated oxidative stress and dysfunction of the mitochondrial electron transport chain. It is believed that ROS is generated either by autooxidation during normal dopamine metabolism or by the action of monoamine oxidase. See, Lev N et al.: Apoptosis and Parkinson's disease; Progress in Neuro-Psychopharmacology and Biological psychiatry 27: 245-50, 2003.

There are many therapies for treating PD. The first effective therapy was carbidopa/levodopa (Sinemet-Bristol Myers Squibb), which controls temor, bradykinesia, balance, and rigidity. Other therapies include dopamine agonists, carbidopa/levodopa therapy, COMT inhibitors, anticholinergics, and MAO inhibitors, such as selegiline/deprenyl. See, Neurodegenerative Disorders: The world market 2002-207; a Visiongain Report; VISIONGAIN™ 2003.

ALS, sometimes called Lou Gehrig's disease, is a rapidly progressive, invariably fatal neurological disease that attacks the nerve cells (neurons) responsible for controlling voluntary muscles. The disease is the most common motor neuron disease and is characterized by the gradual degeneration, and death, of motor neurons. See, Rowland L P, Schneider N A: Amyotrophic lateral sclerosis. N Engl J Med 344: 1688-1700, 2001. Motor neurons are nerve cells located in the brain, brainstem, and spinal cord that serve as controlling units and vital communication links between the nervous system and the voluntary muscles of the body. Messages from motor neurons in the brain (upper motor neurons) are transmitted to motor neurons in the spinal cord (lower motor neurons) and then, to particular muscles. In ALS, both the upper motor neurons and the lower motor neurons degenerate or die, and consequently, cease to send messages to muscles. Unable to function, the muscles gradually weaken, waste away (atrophy), and twitch (fasciculation). Eventually, the ability of the brain to start and control voluntary movement is lost. Most people with ALS die from respiratory failure, usually within 3 to 5 years from the onset of symptoms.

The cause of ALS is not yet known. In some cases of familial ALS, the gene encoding the enzyme superoxide dismustase (SOD) is mutated. See, Rosen D R et al.: Mutations in Cu/Zn superoxide dismutase gene are associated with familial amyotrophic lateral sclerosis. Nature, 362: 59-62, 1993. This enzyme is a powerful antioxidant that protects the body from damage caused by free radicals. Although it is not yet clear how this gene mutation leads to motor neuron degeneration, researchers have theorized that an accumulation of free radicals may result from the faulty functioning of the gene. Currently, the only proven therapy for patients suffering from ALS is Riluzole, which extends survival by approximately 3 months. See, Miller, R. G., Mitchell, J. D., Lyon, M. & Moore, D. H. Riluzole for amyotrophic lateral sclerosis (ALS)/motor neuron disease (MND). Cochrane. Database. Syst. Rev. CD001447 (2002).

Cystic fibrosis (CF) is another disease where molecular chaperones have been implicated. Cystic fibrosis results from defects in the protein Cystic Fibrosis Transmembrane conductance Regulator (CFTR). CFTR normally resides in the plasma (outer) membrane of the cell where it transports chloride. However, in most cases of CF, a specific amino acid in CFTR is deleted, and the resulting mutated protein misfolds and becomes unable to migrate to the plasma membrane. It is believed that the intrinsic biological activity of CFTR can be salvaged by restoring the misfolded protein into a 3-D structure similar to its native structure. For instance, simply lowering the temperature of cells expressing this mutant protein, a condition that tends to stabilize the structure of proteins, allows increased levels of this protein to exit the ER, reach the cell surface, and perform its normal biological function. See, e.g., Dalemans W, Barbry P, Champigny G, Jallat S, Dott K, Dreyer D, Crystal R G, Pavirani A, Lecocq J P, and Lazdunski M. Altered chloride ion channel kinetics associated with the delta F508 cystic fibrosis mutation. Nature 354: 526-528, 1991; Denning G M, Anderson M P, Amara J F, Marshall J, Smith A E, and Welsh M J. Processing of mutant cystic fibrosis transmembrane conductance regulator is temperature-sensitive. Nature 358: 761-764, 1992. Drumm M L, Wilkinson D J, Smit L S, Worrell R T, Strong T V, Frizell R A, Dawson D C, and Collins F S. Chloride conductanceexpressed by delta F508 and other mutant CFTRs in Xenopus oocytes. Science 254: 1797-1799, 1991; each of which is incorporated by reference. It is believed that the proper folding of CRTR can be facilitated by chaperone proteins. Further, chaperones are known to be important mediators in directing proteins to the subcellular compartment in which they belong. It is thus possible that methods of increasing chaperone levels or activity could be therapeutically beneficial to CF patients.

Recently, it has been shown that hydroxyamine compounds are useful in increasing the expression or enhancing the activity of molecular chaperones in cells exposed to a physiological stress. See, e.g., U.S. Pat. No. 6,653,326 and WO 97/16439, both of which are incorporated by reference. These compounds may be used in treatment of conditions, disorders and diseases associated with the function of the chaperone system.

WO 00/50403 reports that the compound N-[2-hydroxy-3-(1-piperidinyl)-propoxy]-pyridine-1-oxide-3-carboximidoyl chloride is capable of lowering insulin resistance. It further reports that this compound is useful in the treatment of chronic diabetic complications, especially retinopathy, neuropathy, nephropathy and the pathological decrease of neuroregeneration in the peripheral nervous system.

WO 01/79174 reports a process for preparing the compound N-[2-hydroxy-3-(1-piperidinyl)-propoxy]-pyridine-1-oxide-3-carboximidoyl chloride.

WO 03/026653 reports that the compound N-[2-hydroxy-3-(1-piperidinyl)-propoxy]-pyridine-1-oxide-3-carboximidoyl chloride in combination with metformin is useful in the treatment of type II diabetes (non-insulin dependent, NIDDM).

WO 05/041965 reports the use of the compound N-[2-hydroxy-3-(1-piperidinyl)-propoxy]-pyridine-1-oxide-3-carboximidoyl chloride in the treatment of certain neurodegenerative diseases.

Unfortunately, the bioavailability of N-[2-hydroxy-3-(1-piperidinyl)-propoxy]-pyridine-1-oxide-3-carboximidoyl chloride is not yet known.

Thus, there remains a great need to identify and develop compounds that can effectively increase the expression and/or enhance the activity of molecular chaperones, and that have good bioavailability. Such compounds would be useful as agents for treating conditions, disorders and diseases where the role of molecular chaperones has been implicated, such as in many neurodegenerative conditions involving the central nervous system.

### SUMMARY OF THE INVENTION

The present invention relates to compositions and methods for treating conditions, disorders or diseases, using hydroxyamine compounds, in particular, N-[2-hydroxy-3-(1-piperidinyl)-propoxy]-pyridine-1-oxide-3-carboximidoyl chloride (Compound I) alone or in combination with at least one additional therapeutic agent wherein the combination shows increased efficacy for treating conditions, diseases or disorders associated with neurodegeneration in the central nervous system. Compound I is represented by:

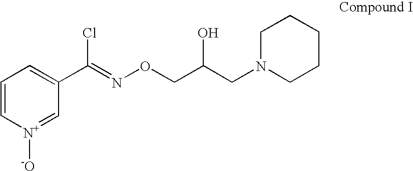

The present invention also provides compositions and methods of treating a condition, disorder or disease in a patient comprising three components: (a) a pharmaceutical composition comprising a therapeutically effective amount of compound (I); (b) an additional therapeutic agent; and (c) a pharmaceutically acceptable carrier; wherein the condition, disorder or disease is associated with neurodegeneration in the central nervous system.

In some embodiments, the disease is ALS. In some embodiments, the disease is Huntington's disease. In some embodiments, the disease is PD. In some embodiments, the disease is stroke. In some embodiments, the disease is cystic fibrosis. Preferred additional therapeutic agents are provided. In some embodiments, the additional agent and Compound I are combined into a single dosage form.

The present invention also provides compositions and methods of treating a condition, disorder, or disease comprising administering Compound II or a pharmaceutical composition comprising a therapeutically effective amount of Compound II and a pharmaceutically acceptable carrier, wherein the condition, disease, or disorder is associated with neurodegeneration in the central nervous system. Compound II is represented below:

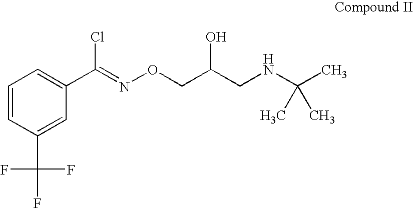

In some embodiments, the disease is ALS. In some embodiments, the disease is Huntington's disease. In some embodiments, the disease is PD. In some embodiments, the disease is stroke. In some embodiments, the disease is cystic fibrosis. Preferred additional therapeutic agents are provided. In some embodiments, an additional agent and Compound II are combined into a single dosage form.

The present invention also provides compositions and methods of treating a condition, disorder, or disease comprising a pharmaceutical compositions comprising three components: (a) a compound of formula (III) or its tautomer compound of formula (IV):

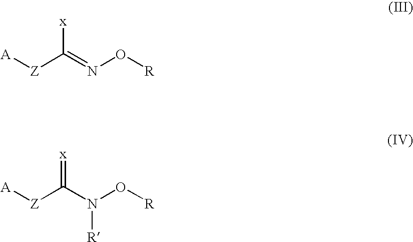

and pharmaceutically acceptable salts thereof, wherein, in each of compounds of formulae (III) and (IV):
A is an alkyl, substituted alkyl, aralkyl, aralkyl substituted in the aryl and/or in the alkyl moiety, aryl, substituted aryl, heteroaryl or substituted heteroaryl group;
Z is a covalent bond, oxygen or ═NR³;
R³ is selected from the group consisting of hydrogen, an alkyl, substituted alkyl, aryl, substituted aryl, aralkyl, or aralkyl substituted in the aryl and/or in the alkyl moiety;
R is an alkyl or substituted alkyl,
X, in compound of formula (III), is halogen or a substituted hydroxy or amino, monosubstituted amino or disubstituted amino group and, in compound of formula (IV), is oxygen, imino or substituted imino group;
R′ is hydrogen, an alkyl, substituted alkyl, aryl, substituted aryl, aralkyl, aralkyl having substituted aryl and/or alkyl moiety, acyl or substituted acyl group;
(b) an additional therapeutic agent; and
(c) a pharmaceutically acceptable carrier, adjuvant or vehicle.

Each of compounds (I), (II), (III) or (IV) may be used alone, together or in combination with one or more additional therapeutic agents for the treatment of a disease, disorder or condition in which molecular chaperones have been implicated. Exemplary conditions, diseases, or disorders which are ameliorated by treatment with these compounds and compositions comprising them are those associated with neurodegeneration in the central nervous system. Preferred additional therapeutic agents are provided.

The present invention thus provides methods for treating a condition, disorder or disease using the compounds or compositions of the present invention. In some embodiments, the disease is a neurodegenerative disease. In other embodiments, the neurodegenerative disease is one of the central nervous system. In some embodiments, the disease is ALS. In some embodiments, the disease is Huntington's disease. In some embodiments, the disease is PD. In some embodiments, the disease is stroke. In some embodiments, the disease is cystic fibrosis.

### BRIEF DESCRIPTION OF THE FIGURES

FIG. 1 depicts that compound I increases spinal HSF1 phosphorylation and increases spinal chaperone protein expression compared to untreated ALS controls.

FIG. 2 depicts that compound I delays disease progression in the ALS transgenic human SOD1^{(G93A)} mouse model.

FIG. 3 depicts Compound I (arimoclomol) single dose pharmacokinetics.

FIG. 4 depicts Compound I (arimoclomol) single dose pharmacokinetics.

FIG. 5 depicts Compound I (arimoclomol) multi-dose serum concentrations.

FIG. 6 depicts the effect of high dose Compound I (arimoclomol) on ALSFRS-R in patients who are not treated with Riluzole.

FIG. 7 depicts the effect of high dose Compound I (arimoclomol) on ALSFRS-R in patients who are also treated with Riluzole.

FIG. 8 depicts the effect of Riluzole on ALSFRS-R in patients who are not treated with Compound I (arimoclomol).

FIG. 9 depicts the effect of Riluzole on ALSFRS-R in patients who are treated with a high dose of Compound I (arimoclomol).

FIG. 10 depicts the effect of the combination of Compound I (arimoclomol) and Riluzole on ALSFRS-R.

FIGS. 11a-b depict the effect of Compound I on Riluzole serum drug levels (a: Cmax; b: AUC).

FIGS. 12a-b depict the effect of Riluzole on Compound I serum drug levels (a: Cmax; b: AUC).

FIG. 13 depicts the ALSFRS-R change from baseline by visit in the open-label study of Compound I against the Celebrex® placebo.

FIG. 14 depicts the Vital Capacity (VC) % predicted maximum change from baseline by visit in the open-label Compound I study against the Celebrex® placebo.

FIG. 15 depicts the weight change from baseline by visit in the open-label Compound I study against the Celebrex® placebo.

FIG. 16 depicts the body-mass index (BMI) change from baseline by visit in the open-label Compound I study against the Celebrex® placebo.

FIG. 17 depicts the ALSFRS-R total with and without Riluzole use in an open-label study of Compound I.

FIG. 18 depicts the ALSFRS-R total change from baseline with and without Riluzole Use in an open-label study of Compound I.

FIG. 19 depicts the Vital Capacity (VC) % predicted maximum with and without Riluzole use in an open-label study of Compound I.

FIG. 20 depicts the Vital Capacity (VC) % predicted maximum change from baseline with and without Riluzole use in an open-label study of Compound I.

### DETAILED DESCRIPTION OF THE INVENTION

In order that the invention herein described may be fully understood, the following detailed description is set forth.

Unless defined otherwise, all technical and scientific terms used herein have the same meaning as those commonly understood by one of ordinary skill in the art to which this invention belongs. Although methods and materials similar or equivalent to those described herein can be used in the practice or testing of the present invention, suitable methods and materials are described below. The materials, methods and examples are illustrative only, and are not intended to be limiting. All publications, patents and other documents mentioned herein are incorporated by reference in their entirety.

Throughout this specification, the word “comprise” or variations such as “comprises” or “comprising” will be understood to imply the inclusion of a stated integer or groups of integers but not the exclusion of any other integer or group of integers.

In order to further define the invention, the following terms and definitions are provided herein.

The term “alkyl” refers to straight or branched, saturated aliphatic hydrocarbon containing 1 to 21 carbon atoms. “Short chain alkyl” refers to an alkyl group containing from 1 to 8 carbon atoms. Examples of short chain alkyl groups include, but are not limited to, methyl, ethyl, propyl, isopropyl, butyl, isobutyl, sec-butyl, pentyl, tert-pentyl, hexyl, heptyl, and octyl groups. Preferably, the short chain alkyl contains from 1 to 6 carbon atoms and is selected from the group consisting of methyl, ethyl, propyl, isopropyl, butyl, isobutyl, sec-butyl, pentyl, tert-pentyl, and hexyl-groups. “Long chain alkyl” refers to an alkyl group containing from 9 to 21 carbon atoms. Examples of long chain alkyl groups include, but are not limited to, nonyl, decyl, undecyl, dodecyl, tridecyl, tetradecyl, pentadecyl, hexadecyl, heptadecyl, octadecyl, nonadecyl, eicosyl and heneicosyl groups. Preferably the long chain alkyl contains from 9 to 17 carbon atoms and is selected from the group consisting of nonyl, decyl, undecyl, dodecyl, tridecyl, tetradecyl, pentadecyl, hexadecyl, and heptadecyl groups.

The term “cycloalkyl” refers to a monocyclic, non-aromatic, hydrocarbon ring system containing 3 to 8 carbon atoms. “Short cycloalkyl chain” refers to a cycloalkyl group containing from 3 to 8 carbon atoms. Examples include, but are not limited to, cyclopropyl, cyclobutyl, cyclopentyl, cyclohexyl, cycloheptyl and cyclooctyl groups. Preferably, the cycloalkyl group contains from 3 to 7 carbon atoms and is selected from the group consisting of cyclopropyl, cyclobutyl, cyclopentyl, cyclohexyl and cycloheptyl.

The term “aryl” refers to a mono- or polycyclic ring system which contains 6, 10, 12 or 14 carbons in which at least one ring of the ring system is aromatic. Examples of aryl ring systems include, but are not limited to, phenyl, naphthyl, pentalenyl, anthracenyl groups. Preferably, the aryl group is phenyl or naphthyl groups.

The term “aralkyl” refers to an alkyl group, wherein one or more hydrogen atoms of the alkyl group is replaced by one or more aryl radical. Examples of aralkyl groups include, but are not limited to, benzyl, benzhydryl, trityl, 1-phenyl-ethyl, 2-phenylethyl, 2-benzhydryl-ethyl, 3-phenylpropyl, 1-methyl-2-phenyl-ethyl, 1-phenylbutyl, 4-tritylbutyl, 1,1-dimethyl-2-phenylethyl, 4-phenylbutyl, 5-phenylpentyl, and 6-phenylhexyl-groups. Preferably, the aralkyl group is a lower alkyl group containing from 1 to 4 carbon atoms, substituted with a phenyl group. Preferred aralkyl groups include, but are not limited to, benzyl, 1-phenylethyl, 2-phenylethyl, and 1-methyl-2-phenylethyl groups.

The term “heterocyclic” refers to a mono ring system which contains 1 to 15 carbon atoms and 1 to 4 heteroatoms, in which the ring system may optionally contain unsaturated bonds but is not aromatic. Heteroatoms are independently sulfur, nitrogen, or oxygen. Examples include, but are not limited to, aziridinyl-, azetidinyl-, oxaziranyl-, pyrrolidinyl-, imidazolidinyl-, pyrazolidinyl-, perhydro-thiazolyl-, perhydro-isoxazolyl-, piperidinyl-, piperazinyl-, perhydro-pyrimidinyl-, perhydro-pyridazinyl-, morpholinyl-, perhidro-1H-azepinyl, oxazolyl, and isoxazolyl, oxadiazolyl (e.g. 1,2,4-oxadiazolyl- and others). Preferably, the heterocyclic ring is a 3-8 membered ring system. More preferably, the heterocyclic ring is a 5-8 membered ring system. More preferably, the heterocyclic ring is 5-6 membered ring, containing 1-2 oxygen atoms and 1-3 N-atoms.

The term “heteroaryl” refers to a mono- or polycyclic ring system which contains 1 to 15 carbon atoms and 1 to 4 heteroatoms, and in which at least one of the rings in the ring system is aromatic. Heteroatoms are sulfur, nitrogen or oxygen. Preferably, the heteroaryl group is an unsaturated, 3-8 membered ring. More preferably, the heteroaryl group is a 5-6 membered, 1-4 N-containing unsaturated hetero-monocyclic group. Examples include, but are not limited to, pyrrolyl, pyrrolinyl, imidazolyl, pyrazolyl, pyridyl group and its N-oxide, primidinyl, pyrazinyl, pyridazinyl, triazolyl, tetrazolyl, and dihydrotriazinyl. Preferably, the heteroaryl group is a polycyclic ring containing 1-5 N-atoms. Examples include, but are not limited to, indolyl, isoindolyl, indolizinyl, benzimidazolyl, quinolyl, isoquinolyl, indazolyl, benzotriazolyl, tetrazolopyridyl, tetrazolopyridazinyl, and dihydro-triazolopyridazinyl. Preferably, the heteroaryl group is a polycyclic ring containing an unsaturated ring, 1-2 oxygen atoms and 1-3 N atoms. Examples include, but are not limited to, benzoxazolyl and benzoxadiazolyl. Preferably, the heteroaryl group is a monocyclic, 3-8 membered ring, more preferably 5-6 membered ring, containing 1-2 sulfur atoms and 1-3 N-atoms. Examples include, but are not limited to, thiazolyl, 1,2-thiazolyl, thiazolinyl, and thiadiazolyl. Preferably, the heteroaryl group is a monocyclic, 3-8 membered ring, more preferably 5-6 membered ring, containing one sulfur atom or one oxygen atom. Examples include, but are not limited to, thienyl and furanyl. Preferably, the heteroaryl is a bicyclic ring containing 1-2 sulfur atoms and 1-3 nitrogen atoms. Examples include, but are not limited to, benzothiazolyl and benzothiadiazolyl.

The term “acyl” group refers to an acyl group which might be a short chain alkanoyl (e.g., formyl, acetyl, propionyl, butyryl and the like), a short chain alkoxy-carbonyl (e.g., methoxy-carbonyl, ethoxy-carbonyl, propoxy-carbonyl, butoxy-carbonyl, tert-butoxy-carbonyl and the like), a short chain alkyl-sulphonyl (e.g., methyl-sulphonyl, ethyl-sulphonyl and the like), aryl-sulphonyl (e.g., phenyl-sulphonyl and the like), aroyl (e.g., benzoyl, naphthoyl and the like), aryl-(short chain alkanoyl) (e.g., phenyl-acetyl, phenyl-propionyl and the like), cyclo-(short chain alkyl)-(short chain alkanoyl) (e.g., cyclohexyl-acetyl and the like), aryl-(short chain alkoxy)-carbonyl (e.g., benzyloxy-carbonyl and the like), aryl-carbamoyl (e.g., phenyl-carbamoyl, naphthyl carbamoyl and the like), cycloalkyl-carbamoyl (e.g., cyclohexyl-carbamoyl and the like), hetero-monocyclic sulphonyl (e.g., thienyl-sulphonyl, furyl-sulphonyl and the like). Acyl group may be optionally substituted with 1-3 substituents as described above.

The term “ω-amino-alkyl” group refers to a short chain alkyl group containing a substituted N-atom in the ω-position of the alkyl chain and in which the alkyl chain is optionally substituted with one or more substituents, preferably with one or two halogen (e.g., chloro, bromo, fluoro, iodo), hydroxyl group or acylated hydroxyl group. Preferably, one or two short chain alkyl groups and the “alkyl” definition is the same as written above. The N-atom in the ω-position of the alkyl chain can be substituted with one or two short chain alkyl substituents, preferably methyl-, ethyl-, tert-butyl- and the like, with cycloalkyl carbamoyl- (e.g., cyclohexyl-carbamoyl- and the like). Preferably, the N-atom can be a part of a saturated heterocyclic group which contains 1-4 nitrogen atoms and is selected from the group consisting of aziridinyl, azetidinyl, oxaziranyl, pyrrolidinyl, imidazolidinyl, pyrazolidinyl, perhydro-thiazolyl, perhydro-isoxazolyl, piperidinyl, piperazinyl, perhydro-pyrimidinyl, perhydro-pyridazinyl, morpholinyl, and perhydro-1H-azepinyl. The N-atom in the ω-position can be substituted with an aryl group (e.g., phenyl and the like), and can be quaternarized by a short chain alkyl substituent or oxidized as well.

The term “halogen” refers to F, Cl, Br, or I.

The term “optionally substituted” aryl or alkyl refers to an aryl- or alkyl group having one or more substituents. Examples of substituents include, but are not limited to, cyano, hydroxyl, short chain alkyl (e.g., methyl, ethyl, propyl, isopropyl, butyl, isobutyl, sec-butyl, pentyl, tert-pentyl, hexyl, heptyl, octyl and the like), short chain alkoxy (e.g., methoxy, ethoxy, propoxy, isopropoxy, butoxy, isobutoxy, sec-butoxy, tert-butoxy, pentyloxy, tert-pentyloxy, hexyloxy and the like), aryl (e.g., phenyl, naphthyl, and the like), nitro, amino, mono-(short chain alkyl)-substituted amino (e.g., methyl, ethyl, propyl, isopropyl, tert-butyl)-amino and the like, di-(short chain alkyl)-substituted amino (e.g., dimethylamino, diethylamino, dipropylamino, diisopropylamino, dibutylamino, dipentylamino, dihexylamino and the like), monohalogen, dihalogen or trihalogen (short chain)-alkyl (e.g., chloromethyl, 2,2-dichloroethyl, trifluoromethyl and the like) or halogen atom (e.g. fluoro-, chloro-, bromio-, and iodine atom).

The term “bioavailable” means that at least some amount of a particular compound is present in the systemic circulation. Formal calculations of oral bioavailability are described in terms of an F value (“Fundamentals of Clinical Pharmacokinetics,” John G. Wegner, Drug Intelligence Publications; Hamilton, Ill. 1975). F values are derived from the ratio of the concentration of the parent drug in the systemic circulation (e.g., plasma) following intravenous administration to the concentration of the parent drug in the systemic circulation after administration by a non-intravenous route (e.g., oral). Therefore, oral bioavailability within the scope of the present invention contemplates the ratio or F value of the amount of parent drug detectable in the plasma after oral administration compared to intravenous administration.

The term “treating” or “treatment” is intended to mean mitigating or alleviating the symptoms a disease in a mammal, such as a human, or the improvement of an ascertainable measurement associated with a disease.

The term “patient” refers to an animal including a mammal (e.g., a human).

The term “pharmaceutically acceptable derivative” refers to any pharmaceutically acceptable salt, ester, or salt of such ester, of a compound of this invention or any other compound which, upon administration to a recipient, is capable of providing (directly or indirectly) a compound of this invention or a metabolite or residue thereof.

### Methods of Treating A Disease Using Compound I

The present invention provides a method of treating a disease, condition or disorder comprising the step of administering a compound (I) or a pharmaceutically acceptable salt thereof:

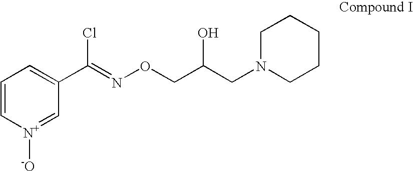

The present invention also provides a method of treating a disease, condition or disorder comprising the step of administering to a patient a pharmaceutical composition comprising a compound (I) or a pharmaceutically acceptable salt thereof and a pharmaceutically acceptable carrier. In some embodiments, the method further comprises administering an additional therapeutic agent. In some embodiments, Compound I and an additional therapeutic agent are combined into a single dosage form.

Compound I exhibits surprisingly good bioavailability in humans. It is rapidly absorbed as an oral formula (low Tmax). It is relatively stable (t1/2=4 h) and is not highly metabolized, but is mostly removed unchanged in urine. It has also been found that this compound crosses the blood:brain barrier in apparent does-dependent fashion, which is surprising given its extremely polar nature (soluble to 14% wt/wt in water). Accordingly, it is well-suited for therapeutic use.

The formula for Compound I is intended to include all stereochemical forms of the compound, including geometric isomers (i.e., E, Z) and optical isomers (i.e., R, S). Single stereochemical isomers as well as enantiomeric and diastereomeric mixtures of the present compounds are within the scope of the invention. Unless otherwise stated, formulas depicted herein are also meant to include compounds which differ only in the presence of one or more isotopically enriched atoms. For example, compounds having the present formulas except for the replacement of a hydrogen by a deuterium or tritium, or the replacement of a carbon by a ¹³C- or ¹⁴C-enriched carbon are within the scope of this invention.

In some embodiments, Compound I has the “R” configuration at the carbon containing the hydroxyl group. In some embodiments, Compound I has the “S” configuration at the carbon containing the hydroxyl group.

In some embodiments, Compound I has the “E” configuration across the carbon-nitrogen double bond. In some embodiments, Compound I has the “Z” configuration across the carbon-nitrogen double bond.

Pharmaceutically acceptable salts of the compounds of this invention include, for example, those derived from pharmaceutically acceptable inorganic and organic acids and bases and amino acids. Examples of suitable acids include hydrochloric, hydrobromic, hydroiodidic, sulfuric, nitric, perchloric, fumaric, maleic, phosphoric, glycolic, lactic, salicylic, succinic, toluene-p-sulfonic, tartaric, acetic, citric, methanesulfonic, formic, benzoic, malonic, naphthalene-2-sulfonic and benzenesulfonic acids. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N—(C1-4 alkyl)₄⁺ salts. Salts derived from amino acids include arginine-salt, glutamic acid salt. In some embodiments, the pharmaceutically acceptable salt is derived from citric acid or maleic acid. In some embodiments, the pharmaceutically acceptable salt is derived from citric acid.

Compound I may be prepared by methods well known to those skilled in the art for analogous compounds. See, e.g., U.S. Pat. No. 6,649,628 and WO 01/79174, both of which are incorporated by reference herein.

Additional therapeutic agents that may be used in the methods of the present invention include, but are not limited to, agents to treat ALS, PD, stroke, AD, Huntington's Disease and cystic fibrosis. Additional therapeutic agents also include, but are not limited to, cholinesterase inhibitors, acetylcholinesterase inhibitors, nerve impulse inhibitors, antioxidants, nonsteroidal anti-inflammatory agents; NMDA antagonists, dopamine agonists, COMT inhibitors, anti-cholinergics, anti-psychotics, anxiolytic agents, dopamine metabolism inhibitors, neuroprotectants, neurotransmitters, neurotransmitter agonists, sedatives, anti-depression agents, neurotransmitter antagonists, stimulants, tranquilizers, and GABA agonists. Other additional therapeutic agents include lumilysergol; benzothialzole, riluzole, phenyl benzothialzole and lifarizine; α-tocopherol.

Suitable acetylcholinesterase inhibitors include galantamine, neostigmine, physostigmine, and edrophonium, and mixtures thereof. Suitable nerve impulse inhibitors include levobupivacaine, lidocaine, prilocalne, mepivacaine, propofol, rapacuronium bromide, ropivacaine, tubocurarine, atracurium, doxaurium, mivacurium, pancuronium, vercuronium, pipecuronium, rocuronium, and mixtures thereof. Suitable anti-cholinergic include acetazolamide, amantadine, carbamazepine, clonazepam, diazepam, divalproex, ethosuximide, ipratropium, lamotrignine acid, levetriacetam, oxcarbazepine, oxitropium, dicycloverine, phenobarbital, phenyloin, pregabalin, primidone, remacemide, trimethadione, topiramate, vigabatrin, zonisamide, and mixtures thereof. Suitable anti-psychotics include amisulpride, aripiprazole bifemelane, bromperidol, clozapine, chlorpromazine, haloperidol, iloperidone loperidone, olanzapine, quetiapine, fluphenazine, fumarate, risperidone, thiothixene, thioridazine, sulpride, ziprasidone, and mixtures thereof. Suitable anxiolytic agents include amitryptiline, atracurium, buspirone, chlorzoxazone, clorazepate, cisatracurium, cyclobenzaprine, eperisone, esopiclone, hydroxyzine, mirtazapine, mivacurium, pagoclone, sulperide, zaleplon, zopiclone, and mixtures thereof. Suitable dopamine metabolism inhibitors include entacapone, lazebemide, selegiline, tolcapone, and mixtures thereof. Suitable agents to treat post stroke sequelae include glatiramer, interferon beta 1A, interferon beta IB, estradiol, progesterone, and mixtures thereof. Suitable neuroprotectants include donepezil, memanine, nimodipine, riluzole, rivastigmine, tacrine, TAK147, xaliproden, and mixtures thereof. Suitable agents to treat Alzheimer's disease include carbidopa, levodopa, tacrine, donezepil (Aricept), rivastigmine (Exelon), galantamine (Reminyl), and mixtures thereof. Suitable neurotransmitters include acetylcholine, serotonin, 5-hydroxytryptamine (5-HT), GABA, glutamate, aspartate, glycine, histamine, epinephrine, norpinephrine, dopamine, adenosine, ATP, nitric oxide, and mixtures thereof. Suitable neurotransmitter agonists include almotriptan, aniracetam, atomoxetine, benserazide, bromocriptine, bupropion, cabergoline, citalopram, clomipramine, desipramine, diazepam, dihydroergotamine, doxepin duloxetine, eletriptan, escitalopram, fluvoxamine, gabapentin, imipramine, moclobemide, naratriptan, nefazodone, nefiracetam acamprosate, nicergoline, nortryptiline, paroxetine, pergolide, pramipexole, rizatriptan, ropinirole, sertraline, sibutramine, sumatriptan, tiagabine, trazodone, venlafaxine, zolmitriptan, and mixtures thereof. Suitable sedatives include dexmedetomidine, eszopiclone, indiplon, zolpidem, zaleplon, and mixtures thereof. Suitable an anti-depression agents include amitriptyline, amoxapine, bupropion, clomipramine, clomipramine, clorgyline, desipramine, doxepin, fluoxetine, imipramine, isocarboxazid, maprotiline, mirtazapine, nefazodone, nortriptyline, paroxetine, phenelzine, protriptyline, sertraline, tranylcypromine, trazodone, venlafaxine, and mixtures thereof. Suitable agents for treating Parkinson's disease include altinicline, amantadine, bomaprine, brasofensine, bromocriptine, budipine, carvidopa, entacapone, ethopropazine, lazabemide, levodopa, memantine, modafinil, pergolide, selegiline, talampanel, tolcapone, trihexyphenidyl, safinamide, droxidopa, rasagline mesylate, cabergoline, pergolide, piribedil, pramipexole, quinagolide, terguride, rotigotine, riluzole, talipexole, piroheptine, bifeprunox, spheramine, sumanirole, lisuride hydrogen maleateor, ropinirole, orphenadrine, and bromocriptine and mixtures thereof. Suitable benzodiazepine antagonists include flumazenil. Suitable neurotransmitter antagonists include deramciclane. Suitable stimulants include amphetamine, dextroamphetamine, dinoprostone, methylphenidate, modafinil, pemoline, and mixtures thereof. Suitable tranquilizers include mesoridazine. Suitable antioxidants include Ginkobiloba extract. Suitable NMDA antagonists include Ebixa (Memantine). Suitable agents for treating ALS include the compounds disclosed in U.S. Pat. No. 5,527,814 and in particular, Riluzole. Suitable GABA agonists include muscimol, progabide, riluzole, baclofen, gabapentin, vigabatrin, valproic acid, tiagabine, lamotrigine, pregabalin, phenyloin, carbamazepine, topiramate.

In some embodiments, the additional therapeutic agent is Riluzole.

Suitable pharmaceutically acceptable carriers that may be used in these pharmaceutical compositions include, but are not limited to, ion exchangers, alumina, aluminum stearate, magnesium stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. In some embodiments, the pharmaceutically acceptable carrier is magnesium stearate.

Compound I and the pharmaceutical compositions of the present invention may be used to treat a patient having a disease, condition or disorder in which molecular chaperones have been implicated. Such diseases include, but are not limited to, neurodegenerative diseases. In some embodiments, the neurodegeneration is in the central nervous system (CNS). In some embodiments, the diseases are selected from the group consisting of stroke, ALS, PD, AD, Huntington's Disease and cystic fibrosis. In some embodiments, the disease is ALS.

### Methods of Treating A Disease Using Compound II

The present invention provides a method of treating a stroke comprising the step of administering compound (II) or a pharmaceutically acceptable salt thereof:

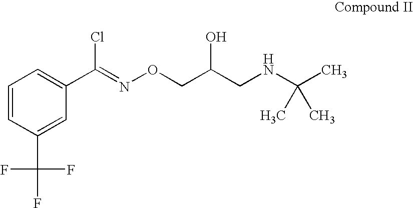

The present invention also provides a method of treating a disease, condition or disorder comprising the step of administering to a patient a pharmaceutical composition comprising compound (II) or a pharmaceutically acceptable salt thereof and a pharmaceutically acceptable carrier. In some embodiments, the method further comprises administering an additional therapeutic agent. In some embodiments, Compound II and an additional therapeutic agent are combined into a single dosage form.

The formula for Compound II is intended to include all stereochemical forms of the compound, including geometric isomers (i.e., E, Z) and optical isomers (i.e., R, S). Single stereochemical isomers as well as enantiomeric and diastereomeric mixtures of the present compounds are within the scope of the invention. Unless otherwise stated, formulas depicted herein are also meant to include compounds which differ only in the presence of one or more isotopically enriched atoms. For example, compounds having the present formulas except for the replacement of a hydrogen by a deuterium or tritium, or the replacement of a carbon by a ¹³C- or ¹⁴C-enriched carbon are within the scope of this invention.

In some embodiments, Compound II has the “R” configuration at the carbon containing the hydroxyl group. In some embodiments, Compound II has the “S” configuration at the carbon containing the hydroxyl group.

In some embodiments, Compound II has the “E” configuration across the carbon-nitrogen double bond. In some embodiments, Compound II has the “Z” configuration across the carbon-nitrogen double bond.

Pharmaceutically acceptable salts of the compounds of this invention include those described above.

Compound II may be prepared by methods well known to those skilled in the art for analogous compounds. See, e.g., U.S. Pat. No. 6,649,628 and WO 01/79174, both of which are incorporated by reference. Compound II may be prepared, for example, using methods described for the preparation of Compound I in the above references, e.g., by starting with CF₃-cyanopyridine instead of CN-pyridine and substituting piperidine with tert-butylamine.

Additional therapeutic agents include those described above.

Suitable pharmaceutically acceptable carriers include those described above.

### Pharmaceutical Compositions Comprising Compounds And Methods of Using Them

The present invention further provides pharmaceutical compositions comprising three components. The first component is a compound represented by formula (III) or its tautomer represented by formula (IV):

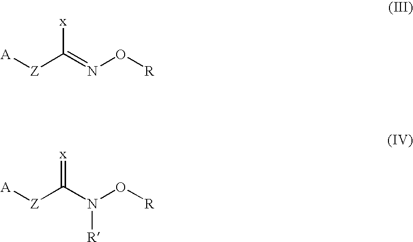

and pharmaceutically acceptable salts thereof,

wherein, in each of compounds of formulae (III) and (IV):

A is an alkyl, substituted alkyl, aralkyl, aralkyl substituted in the aryl and/or in the alkyl moiety, aryl, substituted aryl, heteroaryl or substituted heteroaryl group;

Z is a covalent bond, oxygen or ═NR³;

R³ is selected from the group consisting of hydrogen, an alkyl, substituted alkyl, aryl, substituted aryl, aralkyl, or aralkyl substituted in the aryl and/or in the alkyl moiety;

R is an alkyl or substituted alkyl;

X, in compound of formula (III), is halogen or a substituted hydroxy or amino, monosubstituted amino or disubstituted amino group and, in compound of formula (IV), is oxygen, imino or substituted imino group;

R′ is hydrogen, an alkyl, substituted alkyl, aryl, substituted aryl, aralkyl, aralkyl having substituted aryl and/or alkyl moiety, acyl or substituted acyl group;

and the compounds of formula (I) optionally contain intramolecular ring formulas formed by coupling X and a reactive substituent.

The formulas of compounds of formula (III) and (IV) are intended to include all stereochemical forms of the compound, including geometric isomers (i.e., E, Z) and optical isomers (i.e., R, S). Single stereochemical isomers as well as enantiomeric and diastereomeric mixtures of the present compounds are within the scope of the invention. Unless otherwise stated, formulas depicted herein are also meant to include compounds which differ only in the presence of one or more isotopically enriched atoms. For example, compounds having the present formulas except for the replacement of a hydrogen by a deuterium or tritium, or the replacement of a carbon by a ¹³C- or ¹⁴C-enriched carbon are within the scope of this invention.

In some embodiments, the compound of formula III or IV has the “R” configuration at the carbon containing the hydroxyl group. In some embodiments, the compound of formula III or IV has the “S” configuration at the carbon containing the hydroxyl group.

In some embodiments, the compound of formula III or IV has the “E” configuration across the carbon-nitrogen double bond. In some embodiments, the compound of formula III or IV has the “Z” configuration across the carbon-nitrogen double bond.

In one embodiment, in compounds of formula (III), Z is a covalent bond and X is a halogen. In some aspects of this embodiment, X is chloro or bromo. In some aspects of this embodiment, A is selected from the group consisting of (i) aralkyl or aralkyl having substituted aryl moiety; (ii) aryl or substituted aryl; (iii) naphthyl; (iv) an N-containing heteroaryl group, including those which may be condensed with a benzene ring; (v) an S-containing heteroaryl group and (vi) an O-containing heteroaryl group. In some aspects of this embodiment, A is phenyl alkyl or phenyl alkyl having one or more substituents, preferably alkoxy. In other aspects of this embodiment, A is phenyl or substituted phenyl. In some aspects of this embodiment, A is substituted phenyl containing one or more substituents selected from the group consisting of alkyl, halo, haloalkyl, alkoxy and nitro. In some aspects of this embodiments, A is pyridyl. In further aspects of this embodiment, R is selected from the group consisting of (i) ω-amino-alkyl, (ii) ω-amino-alkyl having mono or disubstituted amino moiety; (iii) ω-amino alkyl having substituted alkyl moiety; (iv) ω-amino alkyl having mono or disubstituted amino moiety and also substituted alkyl moiety. In some aspects of this embodiment, when R is (iv), the alkyl group is substituted with a hydroxy or acyloxy group. In some aspects of this embodiment, the ω-amino-alkyl group is a 3-8 carbon atom alkyl moiety.

Compounds of formula III in which Z is a covalent bond and X is a halogen are disclosed in U.S. Pat. Nos. 5,147,879, 5,328,906, and 5,296,606, all of which are incorporated by reference. These compounds can be prepared by procedures described in the cited U.S. patents, preferably by diazotization of the corresponding X═NH₂ derivatives in the presence of the appropriate hydrohalide. The starting compounds can be obtained by known procedures, e.g., those described in Hungarian Patent No. 177.578 (1976), namely by coupling an amidoxime of formula 1 (R¹═R²═H).

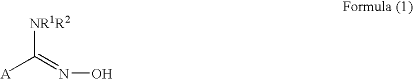

with e.g. a reactive derivative of formula 2:

R-L Formula (2)

in the presence of a base, and can be diazotized usually without isolation or purification. The terminal groups A and R of the compounds can be further amidified or derivatized, as desired.

In another embodiment, in compounds of formula (III), Z is covalent bond and X is a substituted hydroxy group O-Q, wherein Q is an unsubstituted or substituted alkyl or aralkyl group. In one aspect of this embodiment, Q is a straight or branched alkyl. In one aspect of this embodiment, A is aryl or heteroaryl; and R is selected from the group consisting of (i) ω-amino-alkyl, (ii) ω-amino-alkyl having mono or disubstituted amino moiety; (iii) ω-amino alkyl having substituted alkyl moiety; (iv) ω-amino alkyl having mono or disubstituted amino moiety and also substituted alkyl moiety. In some aspects of this embodiment, A is a N-containing heteroaromatic group. In some aspects of this embodiment, when R is (iv), the alkyl group is substituted with a hydroxy or acyloxy group. In some aspects of this embodiment, the ω-amino-alkyl group is a 3-8 carbon atom alkyl moiety.

In another embodiment, in the compound of formula (III), Z is a covalent bond, X is O-Q, Z is a covalent bond, and R is a—CH₂—CH(OH)—R″. The compound is cyclized through the hydroxy group and is represented by formula (III′):

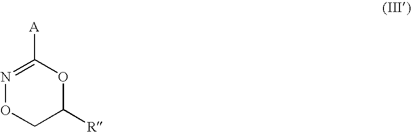

R″ is selected from the group consisting a straight or branched alkyl and a substituted straight or branched alkyl. In some aspects of this embodiment, R″ is ω-amino-alkyl which is optionally substituted on its amino group. In some aspects of this embodiment, R″ is ω-amino-alkyl which is substituted on its amino group with a C₁₋₅ straight or branched alkyl chain. In some aspects, R″ is ω-amino-alkyl mono- or disubstituted on the amino group, wherein the amino-substituents, independently from each other may be one or two straight or branched alkyl or cycloalkyl, or the two amino-substituents, together with the adjacent N-atom form a 3 to 7 heterocyclic ring. In some aspects, the ring is a 5 to 7-membered hetero ring, optionally containing an additional heteroatom. In some aspects, A is selected from the group consisting of phenyl, substituted phenyl, N-containing heteroaryl, substituted N-containing heteroaryl, S-containing heteroaryl, and substituted S-containing heteroaryl.

Compounds of formula III in which Z is a covalent bond and X is a O-Q are disclosed in Hungarian Patent Application No. 2385/1992, which is incorporated by reference. These compounds may be prepared from compounds of formula III in which Z is covalent bond and X is halogen by procedures described in the Hung. Pat. Appln. No. 2385/1992 e.g., by reaction with alkoxides, or by alkaline ring closure for the cyclic compounds of formula (III′).

In another embodiment, in the compounds of formula (III), Z is a covalent bond and X is NR₁R₂, wherein R₁ and R₂ are independently selected from the group consisting of H, straight or branched alkyl, substituted straight or branched alkyl, cycloalkyl, or R₁ and R₂, together with the nitrogen atom to which they are bound, form a saturated ring containing 3 to 7 membered ring. In some aspects of this embodiment, R₁ and R₂ form a saturated 5-7 membered ring. In some aspects of this embodiment, R is selected from the group consisting of (i) ω-amino-alkyl, (ii) ω-amino-alkyl having mono or disubstituted amino moiety; (iii) ω-amino alkyl having substituted alkyl moiety; and (iv) ω-amino alkyl having mono or disubstituted amino moiety and also substituted alkyl moiety. In some aspects of this embodiment, when R is (iv), the alkyl group is substituted with a hydroxy or acyloxy group. In some aspects of this embodiment, the ω-amino-alkyl group is a 3-8 carbon atom alkyl moiety. In some aspects of this embodiment, A is selected from the group consisting of (i) aralkyl or aralkyl having substituted aryl moiety; (ii) aryl or substituted aryl; (iii) naphthyl; (iv) an N-containing heteroaryl group, including those which may be condensed with a benzene ring; (v) an S-containing heteroaryl group and (vi) an O-containing heteroaryl group. In some aspects of this embodiment, A is phenylalkyl or substituted phenylalkyl having one or more substituents. In some aspects of this embodiment, A is phenyl alkyl substituted by one or more alkoxy groups. In some aspects of this embodiment, A is phenyl or substituted phenyl. In some aspects of this embodiment, A is substituted phenyl containing one or more substituents selected from the group consisting of alkyl, halogen, haloalkyl, alkoxy, nitro, and acylamino group. In other aspects of this embodiment, A is pyridyl.

Compounds of formula (III) in which Z is a covalent bond and X is NR¹R² are disclosed in Hungarian Patent No. 177578 (1976) and U.S. Pat. No. 6,653,326, both of which are incorporated by reference. These compounds may be synthesized by alkylation of unsubstituted amidoxime derivatives of compounds of formula (III) (formula (III), wherein R¹═R²═H) with a reactive derivative of compounds of formula (IV) in presence of a base.

In another embodiment, in the compound of formula (III), Z is a covalent bond, X is NR₁R₂, and R is a —CH₂—CH(OH)—R″. The compound is cyclized through the NR₁R₂ group and is represented by formula (III″):

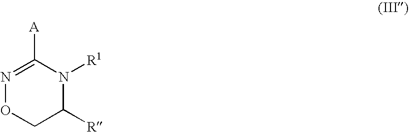

R″ is selected from the group consisting of straight or branched alkyl or a substituted straight or branched alkyl. R¹ is selected from the group consisting of hydrogen, unsubstituted or substituted straight or branched alkyl, cycloalkyl, unsubstituted aralkyl and aralkyl substituted in the aryl- and alkyl moiety. In some aspects of this embodiment, A is selected from the group consisting of (i) aryl or substituted aryl; (ii) naphthyl; (iii) an N-containing heteroaryl group, including those which may be condensed with a benzene ring; (iv) S-containing heteroaryl group; and (v) O-containing heteroaryl group. In some aspects, A is phenyl or substituted phenyl. In some aspects, A is substituted phenyl containing one or more of alkyl, halogen, haloalkyl, alkoxy, amino or nitro group. In further aspects, R″ is selected from the group consisting of (i) ω-amino-alkyl having mono or disubstituted amino moiety, or (ii) ω-amino alkyl having mono or disubstituted amino moiety and also substituted alkyl moiety. In some aspects of this embodiment, the ω-amino-alkyl group is a 3-8 carbon atom alkyl moiety. In some aspects, the ω-amino-alkyl group has disubstituted amino moiety, wherein the substituents, together with the nitrogen atom attached thereto, form a saturated 3-7 membered heterocyclic ring. In some aspects, the ring is 5-7 membered and optionally contains an additional heteroatom. In some aspects, the ω-amino-alkyl groups the amino-substituent is a straight or branched alkyl group or cycloalkyl.

Compounds of formula (III″) may be prepared by ring closure between atoms N(4) —C(5) using the open chain compound of formula (III) in which Z is a covalent bond, X is ═NR¹R², wherein R¹ is as defined in connection with the compounds of the formula (1″) above, R² is H, R is —CH₂—CHY⁵—R″, where Y⁵ is a leaving group, e.g., a halogen atom. Such derivatives may be obtained from the corresponding Y⁵═OH compounds with inorganic halogenating agents, e.g., thionyl chloride or phosphorus pentachloride. The halogenation may be carried out with or without an inert solvent e.g. benzene, chloroform, tetrahydroturane etc., usually by boiling. After removing the excess of the reagent, e.g., by evaporation of the thionyl chloride, the crude halogen derivative may be cyclized—either after or with-out isolation or purification—by treatment with a strong base, e.g., potassium butoxide in t-butanol to give compound of formula (III″), which is finally isolated and purified by standard procedures (extraction, recrystallization, etc.

According to one embodiment, in the compound of formula (III), Z is oxygen and X is O-Q, wherein Q is selected from the group consisting of alkyl, substituted alkyl, aralkyl, and substituted aralkyl having substituted aryl or substituted alkyl moiety. In some aspects of this embodiment, when A is alkyl or substituted alkyl, it contains 1-4 carbon atoms. In some aspects, A is selected from the group consisting of a C₁₋₄ alkyl or substituted alkyl, aralkyl and substituted aralkyl having substituted aryl or substituted alkyl moiety. In some aspects of this embodiment, R is selected from the group consisting of (i) ω-amino-alkyl, (ii) ω-amino-alkyl having mono or disubstituted amino moiety; (iii) ω-amino alkyl having substituted alkyl moiety; and (iv) ω-amino alkyl having mono or disubstituted amino moiety and also substituted alkyl moiety. In some aspects of this embodiment, when R is (iv), the alkyl group is substituted with a hydroxy or acyloxy group.

The compounds of formula (III) in which Z is oxygen and X is O-Q may be prepared by the reaction of O-substituted hydroxylamines of formula 6: (see e.g., Ger. Off. 2,651,083 (1976)) and orthoesters of formula 7:

H₂N—O—R Formula (6)

(COQ)₄. Formula (7)

The condensation may be carried out in the regent itself, as a solvent, preferably by boiling. After evaporation, the product may be isolated by crystallization, if there is an amine function in the side chain R, in the form of acid addition salt.

According to one embodiment, in the compound of formula (III), Z is oxygen, X is NR¹R², and R¹ and R² are independently selected from the group consisting of H, a straight or branched alkyl, a substituted straight or branched alkyl, cycloalkyl, aryl, and substituted aryl, or R¹ and R², together with the nitrogen atom attached thereto, form a saturated ring containing 3 to 7 member saturated ring. In some aspects, R¹ and R² form a 5-7 membered saturated ring. In some aspects of this embodiment, R is selected from the group consisting of (i) ω-amino-alkyl, (ii) ω-amino-alkyl having mono or disubstituted amino moiety; (iii) ω-amino alkyl having substituted alkyl moiety; and (iv) ω-amino alkyl having mono or disubstituted amino moiety and also substituted alkyl moiety. In some aspects of this embodiment, when R is (iv), the alkyl group is substituted with a hydroxy or acyloxy group. In some aspects of this embodiment, the ω-amino-alkyl group is a 3-8 carbon atom alkyl moiety. In some aspects of this embodiment, A is selected from the group consisting of (i) alkyl or substituted alkyl; (iii) aralkyl or aralkyl having substituted aryl and/or substituted alkyl moiety; and (iv) aryl or substituted aryl. In some aspects of this embodiment, A is phenyl or substituted phenyl.

The compounds of formula (III) may be prepared as described hereinbelow, wherein the methods depend on the nature of X, namely whether X is an unsubstituted amino (NH₂) or a substituted amino functionality.

Compounds of formula (III) in which X is NH₂ may be prepared by the addition of hydroxylamine of formula 6 to an organic cyanate of formula A-O—CN (see, e.g., Chem. Ber. 98, 144 (1965)). The reaction may carried out preferably in an inert organic solvent, usually at room temperature. The isolation often requires chromatographic purification.

Compounds of formula (III) in which X is monosubstituted amino group (e.g., NHR¹) may be prepared from known haloformimidates of formula 9:

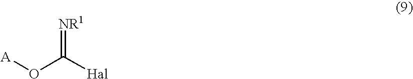

(see e.g. Houben-Weil, “Methoden der Organischen Chemie,” Band E/4, p. 544 (1983) and a compound of formula 6 in the presence of an organic base (e.g., triethylamine) or an inorganic base, such as sodium carbonate in an inert solvent, as benzene, tetrahydroturane, etc., followed by standard work-up and purification procedures.

Compounds of formula (III) in which X is a disubstituted amino group may be prepared by the reaction of a secondary amine of formula 5 with a compound of formula III, where Z is oxygen and X is O-Q (which may be prepared by the method described above):

HNR¹R² Formula (5)

These amination reactions are performed in polar organic solvents, e.g., ethanol, by refluxing, if necessary.

According to another embodiment, in the compound of formula (III), Z is ═NR³, wherein R³ is selected from the group consisting of hydrogen, an alkyl, substituted alkyl, aryl, substituted aryl, aralkyl, and aralkyl having substituted aryl or substituted alkyl moiety; and X is NR¹R², wherein R¹ and R² independently selected from the group consisting of H, a straight or branched alkyl, a substituted straight of branched alkyl, aryl or substituted aryl, cycloalkyl, and R¹ and R², together with the nitrogen atom attached thereto, form a 3 to 7 membered saturated ring.

In some aspects of this embodiment, A is selected from the group consisting of alkyl, substituted alkyl, aralkyl, aralkyl having substituted aryl or substituted alkyl moiety, aryl, and substituted aryl group. In some aspects, R¹ and R² form a 5-7 membered saturated ring. In further aspects of this embodiment, R is selected from the group consisting of (i) ω-amino-alkyl, (ii) ω-amino-alkyl having mono or disubstituted amino moiety; (iii) ω-amino alkyl having substituted alkyl moiety; and (iv) ω-amino alkyl having mono or disubstituted amino moiety and also substituted alkyl moiety. In some aspects of this embodiment, when R is (iv), the alkyl group is substituted with a hydroxy or acyloxy group. In some aspects of this embodiment, the ω-amino-alkyl group is a 3-8 carbon atom alkyl moiety.

Compounds of formula (III) in which Z is NR³ and X is NR¹R², may be prepared by aminolysis of the corresponding isourea derivatives belonging to a group of compounds described above (i.e., compounds of formula (III) in which Z is oxygen and X is NR¹R²) with ammonia or a primary or secondary amine. The reaction may be carried out preferably in a polar solvent, e.g., water or ethanol, using excess of the amine. Alternatively, haloformamides of formula 10 (Houben-Weil “Methoden der Organischen Chemie,” Band E/4, page 553 (1983)) may be reacted with a compound having formula 6 in the presence of an organic or inorganic base to give compounds of this group as well:

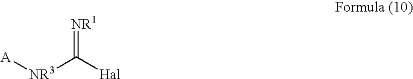

The reaction may be carried out in inert organic solvent, usually at ambient temperature.

Compounds of formula (III) in which R is a group of the formula (b):

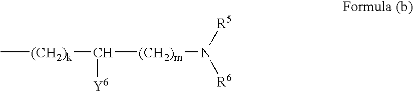

wherein R is acyl, may be prepared by esterifying the corresponding compounds containing hydrogen as R⁷. The alkyl or aryl esters may be obtained by using an acid chloride or anhydride in the presence of a tertiary amine or an inorganic base, preferably in an inert solvent.

According to another embodiment, the present invention provides compounds of formula (IV), which represents the tautomeric form of the compounds of formula (III). In one aspect of this embodiment, in the compound of formula (IV), Z is covalent bond and X is oxygen. In further aspects of this embodiment, A is selected from the group consisting of (i) alkyl, aralkyl or aralkyl having substituted aryl or alkyl moiety; (ii) aryl or substituted aryl; (iii) an N-containing heteroaryl group; and (iv) S-containing heteroaryl group. In some aspects of this embodiment, A is phenyl or substituted phenyl having one or more substitutents. In some aspects of this embodiment, A is substituted phenyl containing one or more substituents selected from the group consisting of alkyl, haloalkyl and alkoxy. In other aspects of this embodiment, A is pyridyl.

In further aspects, R is selected from the group consisting of (i) ω-amino-alkyl, (ii) O-amino-alkyl having mono or disubstituted amino moiety; (iii) ω-amino alkyl having substituted alkyl moiety; and (iv) ω-amino alkyl having mono or disubstituted amino moiety and also substituted alkyl moiety. In some aspects of this embodiment, when R is (iv), the alkyl group is substituted with a hydroxy or acyloxy group. In some aspects of this embodiment, the ω-amino-alkyl group is a 3-8 carbon atom alkyl moiety.

In further aspects, R′ is selected from the group consisting of hydrogen, an alkyl, substituted alkyl, aryl, substituted aryl, aralkyl, and aralkyl having substituted aryl or alkyl moiety.

Compounds belonging to this group are disclosed in the Hungarian Patent Application No. 2385/1992, which is incorporated by reference. These compounds may be prepared according to the methods described therein, most preferably, they can be obtained by acylation of O-substituted hydroxylamine derivatives having formula 6 (see also, e.g., Ger. Off. 2,651,083 (1976)) with an acid chloride having formula 11:

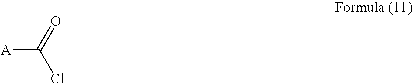

This route may also be employed for the preparation compounds in which R′ is other than hydrogen, using compound of formula 12—instead of formula 6—as starting material:

R¹HN—O—R Formula (12)

According to another embodiment, in compounds of formula (IV), Z is a chemical bond; X is ═NR⁴, wherein R⁴ is selected from the group consisting of H, an alkyl, substituted alkyl, aryl, substituted aryl, aralkyl, aralkyl having substituted aryl or substituted alkyl group, cycloalkyl; and R⁴ is selected from the group consisting of alkyl, substituted alkyl, aryl, substituted aryl, aralkyl, and aralkyl having substituted aryl or substituted alklyl moiety. In some aspects of this embodiment, A is (i) aralkyl or aralkyl having substituted aryl moiety; (ii) aryl or substituted aryl; (iii) naphthyl; (iv) an N-containing heteroaryl group; and (v) S-containing heteroaryl group. In some aspects of this embodiment, A is phenyl alkyl or phenyl alkyl having one or more substituents. In some aspects of this embodiment, A is phenyl alkyl substituted by one or more alkoxy groups. In some aspects of this embodiment, A is phenyl or substituted phenyl. In some aspects of this embodiment, A is substituted phenyl containing one or more substituents selected from the group consisting of alkyl, haloalkyl and nitro. In other aspects of this embodiment, A is pyridyl.

In some embodiments, R is selected from the group consisting of (i) ω-amino-alkyl, (ii) co-amino-alkyl having mono or disubstituted amino moiety; (iii) ω-amino alkyl having substituted alkyl moiety; and (iv) ω-amino alkyl having mono or disubstituted amino moiety and also substituted alkyl moiety. In some aspects of this embodiment, when R is (iv), the alkyl group is substituted with a hydroxy or acyloxy group. In some aspects of this embodiment, the ω-amino-alkyl group is a 3-8 carbon atom alkyl moiety.

These compounds may be prepared either by O-alkylation of a N,N′-disubstituted amidoxime of formula 13:

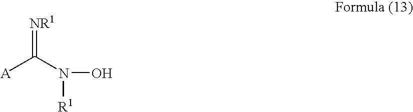

with a chemical compound having formula 2 (for the reaction conditions, see preparation of compounds of formula (III), wherein Z is covalent bond and X is NR¹R²), or by O-acylating an N,O-disubstituted hydroxylamine of the formula 12 with an imidoyl halide of the formula (16):

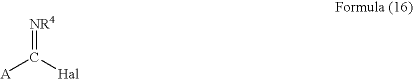

The reaction may be carried out in an inert solvent, preferably in the presence of an organic or inorganic acid scavenger.

The compounds wherein R is a group of the formula (b) wherein R is acyl, may be prepared by esterifying the corresponding compounds containing hydrogen as R⁷. The alkyl or aryl esters may be obtained by using an acid chloride or anhydride in the presence of a tertiary amine or an inorganic base, preferably in an inert solvent.

According to one embodiment, in compounds of formula (IV), Z is oxygen and X is oxygen. In some aspects of this embodiment, A is selected from the group consisting of alkyl, substituted alkyl, aralkyl, and aralkyl with substituted aryl or alkyl moiety. In some aspects, R is selected from the group consisting of (i) ω-amino-alkyl, (ii) ω-amino-alkyl having mono or disubstituted amino moiety; (iii) ω-amino alkyl having substituted alkyl moiety; and (iv) ω-amino alkyl having mono or disubstituted amino moiety A) and also substituted alkyl moiety. In some aspects of this embodiment, when R is (iv), the alkyl group is substituted with a hydroxy or acyloxy group. In some aspects of this embodiment, the ω-amino-alkyl group is a 3-8 carbon atom alkyl moiety. In some aspects of this embodiment, R′ is selected from the group consisting of hydrogen, an alkyl, substituted alkyl, aryl, substituted aryl, aralkyl, and aralkyl with substituted aryl or alkyl moiety.

According to this embodiment, the compounds are disclosed in Hungarian Patent Application No. 1756/95 (filed Jun. 15, 1995), which is incorporated by reference. These compounds may be prepared by acylation of a hydroxylamine having, formula (6) or formula (12) with a chloroformate having formula (14), in a similar manner as with the simple acid chlorides, as described for the synthesis of compounds of formula (IV) wherein Z is covalent bond and X is oxygen. The reaction requires the presence of a base, inorganic or organic, and may be performed in an inert solvent, e.g., in chloroform. The side-product salt is removed, e.g., by extraction with water, and the product is isolated from the organic solution.

In yet another embodiment, in the compounds of formula (IV), Z is oxygen; X is ═NR⁴, wherein R⁴ is selected from the group consisting of alkyl, substituted alkyl, aralkyl, substituted aralkyl having substituted aryl or substituted alkyl group, aryl, substituted aryl, heteroaryl and substituted heteroaryl group. In some aspects of this embodiment, A is selected from the group consisting of alkyl, substituted alkyl, aryl, substituted aryl, aralkyl and aralkyl with substituted aryl or alkyl moiety. In some aspects of this embodiment, A is an unsubstituted or substituted phenyl.

In some aspects of this embodiment, R is ω-aminoalkyl, which suitably contains a hydroxy or acyloxy group in the alkyl chain, and is optionally substituted on the amine nitrogen, wherein the alkyl chain of the ω-aminoalkyl group preferably contains 3 to 8 carbon atoms. In some aspects of this embodiment, R′ is selected from the group consisting of alkyl, aryl or aralkyl which groups may be unsubstituted or substituted.

According to this embodiment, these compounds of formula (III), wherein Z is oxygen and X is NR¹R² may be prepared, similarly from haloformimidates having formula (9) and a chemical compound having formula (12), in the presence of an organic base (e.g., triethylamine) or inorganic base, e.g sodium carbonate in an inert solvent, as benzene, tetrahydrofurane etc., followed by standard work-up and purification procedures.

In another embodiment, in the compounds of formula (IV), Z is ═NR³, wherein R³ is selected from the group consisting of hydrogen, an alkyl, substituted alkyl, aryl, substituted aryl, aralkyl, and aralkyl having substituted aryl or substituted alkyl moiety; and X is oxygen. In some aspects of this embodiment, A is selected from the group consisting of (i) aralkyl or aralkyl having substituted alkyl or aryl moiety; (ii) aryl or substituted aryl, (iii) an N-containing heteroaryl group; (iv) an alkyl or substituted alkyl, straight or branched; and (v) a cycloalkyl group. In some aspects of this embodiment, A is phenyl alkyl or phenyl alkyl having one or more substituents. In some aspects of this embodiment, A is phenyl or substituted phenyl. In some aspects of this embodiment, A is substituted phenyl containing one or more substituents selected from the group consisting of alkyl, alkoxy, halogen, haloalkyl and nitro group. In other aspects of this embodiment, when a is (iv), the alkyl group contains 4 to 12 carbon atoms.

In some aspects of this embodiment, R is selected from the group consisting of (i) ω-amino-alkyl, (ii) ω-amino-alkyl having mono or disubstituted amino moiety; (iii) ω-amino alkyl having substituted alkyl moiety; and (iv) ω-amino alkyl having mono or disubstituted amino moiety and also substituted alkyl moiety. In some aspects of this embodiment, when R is (iv), the alkyl group is substituted with a hydroxy or acyloxy group. In some aspects of this embodiment, the ω-amino-alkyl group is a 3-8 carbon atom alkyl moiety. In some aspects, R′ is selected from the group consisting of hydrogen, an alkyl, substituted alkyl, aralkyl, aralkyl having substituted aryl or alkyl moiety, aryl, substituted aryl, acyl and substituted acyl group.

According to this embodiment, these compounds are disclosed in a Hungarian Patent Application No. 1756/95, which is incorporated by reference, and may be prepared by the reaction of a hydroxylamine compound having formula (6) or formula (12) with an isocyanate having formula (15):

A-N═C═O Formula (15)

in an inert solvent, usually by simple stirring of the mixture at room temperature for 2-24 hours. Finally, the products may be isolated, following evaporation of the solvent. In some aspects, the product may be isolated by crystallization.

In another embodiment, in the compounds of formula (IV), Z is ═NR³, wherein R³ is selected from the group consisting of hydrogen, an alkyl, substituted alkyl, aryl, substituted aryl, aralkyl, and aralkyl having substituted aryl or substituted alkyl moiety; X is ═NR⁴, wherein R⁴ is selected from the group consisting of H, an alkyl, substituted alkyl, aryl, substituted aryl, aralkyl, aralkyl having substituted aryl or substituted alkyl group, and cycloalkyl; and R′ is selected from the group consisting of alkyl, substituted alkyl, aralkyl, substituted aralkyl having substituted aryl or substituted alkyl moiety, aryl and substituted aryl. In some aspects of this embodiment, R³ is selected from the group consisting of hydrogen, alkyl and substituted alkyl; R⁴ is hydrogen or an aryl group; and A is selected from the group consisting of alkyl, substituted alkyl, aryl and substituted aryl, or aralkyl, which may be substituted in the aryl and/or alkyl moiety. In further aspects, R is selected from the group consisting of (i) ω-amino-alkyl, (ii) ω-amino-alkyl having mono or disubstituted amino moiety; (iii) ω-amino alkyl having substituted alkyl moiety; and (iv) co-amino alkyl having mono or disubstituted amino moiety and also substituted alkyl moiety. In some aspects of this embodiment, when R is (iv), the alkyl group is substituted with a hydroxy or acyloxy group. In some aspects of this embodiment, the ω-amino-alkyl group is a 3-8 carbon atom alkyl moiety.

According to this embodiment, the compounds may be prepared by aminolysis of the corresponding isourea derivatives (compounds of formula (IV), wherein Z is oxygen and X is NR⁴) with a primary or secondary amine or ammonia. The reaction may be carried out preferably in a polar solvent, e.g., water or ethanol, using an excess of the amine. Alternatively, the compounds may be prepared by reacting haloformamidines of formula (10) with a compound of formula (12) in the presence of an organic or inorganic base in inert solvents, usually at their boiling point.

According to one embodiment, the present invention provides compounds of formula (III) in which X is halogen; Z is a chemical bond and A is a group of the formula (a) wherein Y¹ is selected from the group consisting of halo, alkoxy, a nitro group and a haloalkyl group; and n is selected from the group consisting of 1, 2, and 3; or O-containing heteroaryl, S-containing heteroaryl, or N-containing heteroaryl group which may be condensed with a benzene ring; and R is a group having formula (b), wherein R⁵ and R⁶, independently from each other, are selected from the group consisting of H, a straight or branched alkyl, and cycloalkyl, or R⁵ and R⁶, when taken together with the nitrogen atom attached thereto, form a 3 to 7; Y⁶ is —OR⁷ wherein R⁷ is H or an acyl group; k is 1, 2 or 3; and m is 1, 2, or 3, with the proviso, that when A is pyridyl or naphthyl, or a group of the formula (a) wherein Y¹ is halo or alkoxy, then R⁷ is other than H. These compounds may optionally contain as A an N-containing heteroaromatic group with N-quaternary C₁₋₄ alkyl or the oxide of the said N-containing heteroaromatic group and/or an R wherein the ring formed by the terminal groups R⁶ and R⁷ is an N-quaternary or N-oxidized saturated heterocyclic ring.

In some aspects of this embodiment, X is chloro or bromo. In some aspects of this embodiment, Y¹ is haloalkyl containing 1-4 carbon atoms. In other aspects, Y¹ is selected from the group consisting of furyl, thienyl, piridyl, quinolyl, and isoquinolyl. In some aspects of this embodiment, R⁵ and R⁶, independently from each other, is substituted straight or branched alkyl. In some aspects, R⁵ and R⁶ is C₁₋₄ alkyl. In other aspects, when R⁵ and R⁶ together with the nitrogen atom attached thereto form a 3 to 7, the resulting ring is a 5 to 7-membered saturated heterocyclic ring. In some aspects, R⁷ is selected from the group consisting of alkyl carbonyl, substituted alkyl carbonyl, aryl carbonyl or substituted aryl carbonyl, and aminoacyl or substituted aminoacyl.

In some aspects of this embodiment, A is a group of the formula (a) wherein Y¹ is trifluoromethyl. In some aspects of this embodiment, X is halo, A is pyridyl, Z is a chemical bond, and R is the group of the formula (b) wherein R⁵ and R⁶ independently from each other are selected from the group consisting of H, straight or branched alkyl, and cycloalkyl, or R⁵ and R⁶ together with the adjacent N atom form a 3 to 7-membered, Y⁶ is —OR⁷, wherein R⁷ is aminoacyl, k is 1, 2 or 3 and m is 1, 2 or 3. In some aspects, R⁵ and R⁶ independently from each other are C₁₋₄ alkyl or cycloalkyl. In other aspects, R⁵ and R⁶ together with the adjacent N atom form y 5 to 7-membered heterocyclic ring. According to each aspect of this embodiment, the compounds may be optically active.

According to this embodiment, these compounds may be prepared using procedures that are analogous to those described in U.S. Pat. Nos. 5,147,879: 5,398,906; and 5,996,606, all of which are incorporated by reference. For example, compounds in which both R⁵ and R⁶ are other than hydrogen, may be prepared by the diazotization of the corresponding NH₂ derivatives (i.e., the compound of formula (III) in which Z is covalent bond and X is NH₂) in the presence of the appropriate hydrogen halide, similarly to the procedure described in U.S. Pat. Nos. 5,147,879; 5,328,906, and 5,296,606. The starting compounds may be obtained also by a known procedure, e.g., those described in Hungarian Patent No. 177578, which is incorporated by reference, namely by coupling an amidoxime having formula 1, wherein R¹ and R² of formula 1 is H, with e.g., a reactive derivative having formula 2 in the presence of a base, and may be diazotized usually without isolation or purification.

Alternatively, for compounds in which R⁷ is H and m is 1, the compounds may be prepared by the reaction of an oxyrane of formula 3 and amine of formula 4. This procedure also may be used for the preparation of compound in which R⁵ is H.

Alternatively, for compounds in which R is represented by formula (b) and R⁷ is an acyl group, the compounds may be prepared by the esterification of the corresponding compounds in which R⁷ is H. Alkyl or aryl esters may be obtained with an acid chloride or anhydride in the presence of a tertiary amine or an inorganic base, preferably in an inert solvent, or in certain cases by the Schotten-Bauman procedure using aqueous inorganic base in a two-phase system. For the preparation of the aminoacyl esters, carboxyl-activated N-protected amino acid derivatives (e.g., active esters) may be used as reagents in procedures basically known from the peptide chemistry. This coupling also requires the presence of a base (e.g., triethylamine). The isolation and purification of the products may be performed by using standard preparative techniques; the final preparation may often be in the form of a salt with appropriate inorganic or organic acids. Starting from chiral amino acids, the products may be frequently diastereomers, possessing the second chiral center in the R group. During the isolation, these diastereomers often may separate, and the product may be obtained in stereo-pure form.

In yet another embodiment of compounds of formula (III), Z is a chemical bond, X is halo; A is a group of the formula (c) and R is a group of the formula (d):

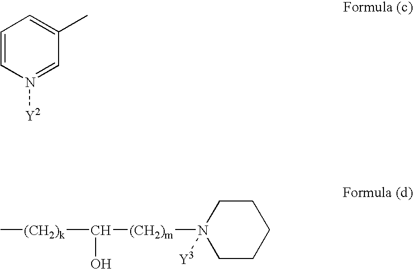

one or both of Y² and Y³ from which at least one must be present in the molecule, are oxygen, or an alkyl or substituted alkyl having 1-4 carbon atoms, k is 1, 2, or 3; and m is 1, 2, or 3. Y² and Y³ are attached by the dotted line. In some aspects of this embodiment, X is chloro or bromo. When the compound is a mono- or bivalent cation, the anion thereof is one or two halide ions. In some aspects of this embodiment, the anion is an iodide ion.

According to this embodiment, the compounds may be prepared by chemical modifications of the terminal pyridine and/or piperidine groups in their unsubstituted precurors, e.g., by N-oxidation or quaternerization. In some aspects of this embodiment, the compounds may be prepared by oxidation with peracids in inert solvents. In further aspects of this embodiment, the peracid is a substituted perbenzoic acid. In further aspects of this embodiment, the inert solvent is chloroform or dichloromethane. If both oxidizable groups are present, mono- or dioxides may form depending on the quantity of the reagent used. At the end of the oxidation reaction, the excess reagent is decomposed and the product is isolated by evaporation. In other aspects of this embodiment, the compounds may prepared by quaternerization. In some aspects of this embodiment, the compounds may be prepared by quaternization with alkyl halides. In some aspects of this embodiment, the alkyl halide is methyliodide. In further aspects of this embodiment, the compound may be prepared by refluxing the reagent in a suitable solvent. In some aspects, the solvent is acetone. In some aspects of this embodiment, the compound is insoluble in the medium, and may be isolated by simple filtration.

In yet another embodiment of compounds of formula (III), Z is a chemical bond, A is selected from the group consisting of aralkyl, substituted aralkyl, phenyl, substituted phenyl having one or more substituents, a N-containing heteroaryl group, which may be condensed with benzene ring, and a sulfur containing heteroaromatic group; X is —NR¹R², wherein R¹ and R², independently from each other, are selected from the group consisting of H, a straight or branched alkyl, a substituted straight or branched alkyl, cycloalkyl and R¹ and R² taken together with the nitrogen atom attached thereto may form a 3 to 7; R is a group of the formula (e)

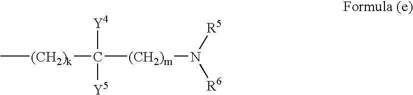

wherein R⁵ and R⁶, independently from each other, are selected from the group consisting of H, a straight or branched alkyl, or a substituted straight or branched alkyl, or cycloalkyl, or R⁵ and R⁶ taken together with the nitrogen atom attached thereto form a 3-7, which may contain additional hetero atoms and substituents; Y⁴ is selected from the group consisting of H, alkyl and substituted alkyl having 1-4 carbon atoms; Y⁵ is selected from the group consisting of H, alkyl and substituted alky; having 1-4 carbon atoms, or OR⁷, wherein R⁷ is H or an acyl; k is 1, 2, or 3; and m is 1, 2, or 3, with the proviso that when A is phenyl which is unsubstituted or substituted with halogen or alkoxy, or phenylalkyl substituted with alkoxy, or a pyridyl group, and R⁷ is H, then at least one of R¹ and R² is other than H, or when A is phenyl which is unsubstituted or substituted with halogen or alkoxy phenylalkyl substituted with alkoxy, or pyridyl, and R¹ and R² are each H, then R⁷ is other than H.

In some aspects of this embodiment, A is phenylalkyl or phenyl. In some aspects, when A is phenylalkyl, the phenyl may be substituted with one or more alkoxy groups. In some aspects, the alkoxy group has 1 to 4 carbon atoms. In other aspects, A is substituted phenyl having one or more substituents. In some aspects, the substituent groups are selected from the group consisting of an alkyl, preferably alkyl or haloalkyl having 1 to 4 carbon atom, halo, acylamino or nitro group. In other aspects, A is selected from the group consisting of pyrrolyl, pyridyl, isoquinolyl, quinolyl and thienyl. In some aspects, when A is a heteroaryl group, it may be substituted with one or more alkyl, preferably alkyl having 1 to 4 carbon atoms.

In some aspects of this embodiment, R¹ and R², independently from each other, are alkyl having 1 to 6 carbon atoms. In other aspects, when R¹ and R² are taken together with the nitrogen atom attached thereto form a ring, the ring is a 5-7 membered saturated hetero ring.

In some aspects of this embodiment, R⁵ and R⁶, independently from each other, are alkyl having 1 to 4 carbon atoms. In other aspects, when R⁵ and R⁶ are taken together with the nitrogen atom attached thereto to form a ring, the ring is a 5-7 membered saturated hetero ring, which may contain additional hetero atoms and substituents. In this aspect, the substituents may be alkyl having 1 to 4 carbon atoms.

According to this embodiment, compounds wherein X is NH₂ may be prepared, similarly to the above-mentioned procedure, by the reaction of the corresponding compound of formula 1, wherein R¹ and R² of formula 1 are H, with a compound of formula 2. The alkylating agent of formula 2 may contain hydroxyl and/or amino substituents. The reaction requires the presence of an inorganic or organic base, in a preferable manner alcoholic alcoholate solution is used as medium and base. The compounds may be isolated as a salt with a suitable organic or inorganic acid.

According to this embodiment, compounds wherein R¹ and R², one or both of them are other than H may be prepared by two methods. In the first method, an amidoxime of formula 1, having the required substituents R¹ and/or R², may be reacted with a reactive compound of formula 2, similarly to the procedure described in the previous paragraph. The substituted amidoximes of formula 1, used as starting materials, are known from the literature. See, e.g., Chem. Rev. 62, 155-183 (1962), which is incorporated by reference.

In the second method, substitution of the halogen atoms in the compounds of formula (IIII), wherein Z is covalent bond and X is halogen, by an amine of formula (5) may result in similar compounds as well. In the case of derivatives bearing an OH substituent in the R group (Y⁴═OH), this hydroxyl group has to be protected before, and deprotected after the substitution reaction, otherwise formation of the cyclic derivatives of formula (I′) is favored. For the protection, acetyl type protecting groups, e.g., tetrahydropyranyl group, have proven most satisfactory. The protection may be carried out by the reaction of the unprotected compound with dihydropyrane, followed by the halogen/amine displacement, which usually requires refluxing in a solvent, e.g., in alcohol. The deprotection of the product, finally, may be accomplished by acidic treatment, e.g., by boiling the ethanolic solution in the presence of e.g., p-toluenesulphonic acid.

According to another embodiment, compounds of formula (III) include those wherein Y⁵ is an acyloxy group. They can be prepared by acylation of the corresponding compound in which Y⁵ is OH, which are either known from the literature (e.g, Haung. Patent No. 177578) or described in the present invention. The reactions may be accomplished identically to what is described for the analogous halo derivatives, wherein R⁷ is an acyl group.

According to another embodiment, compounds of formula (III) also include those wherein Z is oxygen or an ═NR³ group wherein R³ is an unsubstituted or substituted alkyl group; X is —NR¹R², wherein R¹ and R², independently from each other, are selected from the group consisting of hydrogen, unsubstituted or substituted straight or branched alkyl, unsubstituted or substituted aryl, and unsubstituted or substituted aralkyl group, or R¹ and R² are taken together with the nitrogen atom attached thereto to form a 3 to 7 membered saturated heterocyclic ring which optionally contains one or more hetero atoms. According to this embodiment, A is selected from the group consisting of an unsubstituted or substituted alkyl, an unsubstituted or substituted aryl, and unsubstituted or substituted aralkyl group. Further according to this embodiment, R is a group of the formula (b) wherein R⁵ and R⁶, independently from each other are selected from the group consisting of H, straight or branched alkyl, and cycloalkyl, or R⁵ and R⁶ together with the N-atom attached thereto form a 3 to 7-membered saturated heterocyclic ring. According to this embodiment, Y⁶ is H or —OR⁷, wherein R⁷ is H or acyl, k is 1, 2 or 3 and m is 1, 2 or 3.

In one aspect of this embodiment, R¹ and R², independently from each other, are phenyl. In other aspects, when R¹ and R² are taken together with the nitrogen atom attached thereto to form a ring, the ring is a 5 to 7 membered saturated heterocyclic ring which optionally contains one or more heteroatoms. According to some aspects, A is phenyl or substituted phenyl group. According to some aspects, R⁵ and R⁶, independently from each other, are C₁₋₄ alkyl. Alternatively according to some aspects, R⁵ and R⁶ together with the N-atom attached thereto, form a 3 to 7-membered ring, the ring is a 5 to 7-membered saturated heterocyclic ring. According to some aspects, R⁷ is unsubstituted or substituted alkylcarbonyl or arylcarbonyl.

According to another embodiment, compounds of formula (III) also include those wherein Z is oxygen and X is —OR, wherein Q is an unsubstituted or substituted alkyl or unsubstituted or substituted aralkyl group, A is an unsubstituted or substituted alkoxy group or an unsubstituted or substituted aralkyl group and R is a group of the formula (b), wherein R⁵ and R⁶, independently from each other, are selected from the group consisting of H, straight or branched alkyl, and cycloalkyl, or R⁵ and R⁶, together with the N-atom attached thereto, form a 3 to 7-membered saturated heterocyclic ring, Y⁶ is H or —OR⁷, wherein R⁷ is H or acyl, k is 1, 2 or 3 and m is 1, 2 or 3.

In some aspects of this embodiment, R⁵ and R⁶, independently from each other, are C₁₋₄ alkyl. In other aspects, R⁵ and R⁶, when taken together with the N atom attached thereto form a 3 to 7-membered, the ring is a 5 to 7-membered heterocyclic ring. In some aspects, R⁷ is unsubstituted or substituted alkylcarbonyl or arylcarbonyl.

According to another embodiment, compounds of formula (III) also include those wherein A is selected from the group consisting of unsubstituted or substituted aryl, N-containing heteroaromatic group and S-containing heteroaromatic group, Z is a chemical bond, X is —OQ wherein Q is C₁₋₄ alkyl and R is a group of the formula (b), wherein R⁵ and R⁶, independently from each other are selected from the group consisting of H, straight or branched alkyl, and cycloalkyl, or R⁵ and R⁶, when taken together with the N atom attached thereto form a 3 to 7-membered heterocyclic ring, Y⁶ is H, k is 1, 2 or 3 and m is 1, 2 or 3.

In some aspects of this embodiment, A is phenyl. In other aspects, A is pyridyl. In some aspects of this embodiment, R⁵ and R⁶, independently from each other, are C₁₋₄ alkyl. In other aspects, R⁵ and R⁶ are taken together with the N atom attached thereto to form a 5 to 7-membered heterocyclic ring.

According to this embodiment, these compounds may prepared by the reaction of the corresponding compound of formula (III) wherein X is halo and the corresponding alcoholates, preferably in an alcohol corresponding to the alcoholate, preferably by refluxing. The reaction mixture may be treated with methods known in the art and the product may be isolated by chromatography or salt-forming.

According to yet another embodiment, compounds of formula (IV) include those wherein X is oxygen, A is selected from the group consisting of C₁₋₂₀ straight or branched alkyl, unsubstituted or substituted aryl, unsubstituted or substituted aralkyl, naphthyl and N-containing heteroaromatic group, Z is a chemical bond, R′ is selected from the group consisting of H, C₁₋₄ alkyl and aralkyl, Z is a group of the formula (b), wherein R⁵ and R⁶ independently from each other, are selected from the group consisting of H, straight or branched alkyl, and cycloalkyl, or R⁵ and R⁶ are taken together with the N atom attached thereto to form a 3 to 7-membered heterocyclic ring, Y⁶ is H or —OR⁷, R⁷ is H, k is 1, 2 or 3 and m is 1, 2 or 3, with the proviso, that when A is other than alkyl and R¹ is H, Y⁶ is H.

In some aspects of this embodiment, A is phenyl or halophenyl. In other aspects, A is pyridyl. In some aspects of this embodiment, R′ is phenylalkyl. In some aspects of this embodiment, R⁵ and R⁶ independently from each other, are C₁₋₄ alkyl. In other aspects, R⁵ and R⁶ are taken together with the N atom attached thereto to form a 5 to 7-membered heterocyclic ring.

According to yet another embodiment, compounds of formula (IV) also include those wherein Z is selected from the group consisting of a covalent bond, oxygen and an ═NR³ group, wherein R³ is hydrogen or an unsubstituted or substituted alkyl group, X is ═NR⁴, wherein R⁴ is selected from the group consisting of hydrogen, an unsubstituted or substituted alkyl, an unsubstituted or substituted aryl, and a substituted or unsubstituted aralkyl. According to this embodiment, A is selected from the group consisting of an unsubstituted or substituted alkyl, an unsubstituted or substituted aryl, an unsubstituted or substituted aralkyl, and cycloalkyl, R′ is selected from the group consisting of an unsubstituted or substituted alkyl, an unsubstituted or substituted aryl, and an unsubstituted or substituted aralkyl, R is a group of the formula (b), wherein R⁵ and R⁶, independently from each other, are selected from the group consisting of H, straight or branched alkyl, or R⁵ and R⁶ are taken together with the N atom attached thereto to form 3 to 7-membered heterocyclic ring, Y⁶ is H or —OR⁷, R⁷ is H or acyl, k is 1, 2 or 3 and m is 1, 2 or 3.

In some aspects of this embodiment, R⁴ is phenyl or phenylalkyl. In some aspects of this embodiment, A is selected from the group consisting of phenyl, substituted phenyl, and phenylalkyl. In some aspects of this embodiment, R′ is phenyl or pheylalkyl. In some aspects of this embodiment, R⁵ and R⁶, independently from each other, are C₁₋₄ alkyl. In other aspects, R⁵ and R⁶ are taken together with the N atom attached thereto to a form 5 to 7-membered heterocyclic ring. In some aspects of this embodiment, R⁷ is unsubstituted or substituted alkylcarbonyl or arylcarbonyl.

According to yet another embodiment, compounds of formula (IV) also include those wherein X is oxygen, A is unsubstituted or substituted alkyl, unsubstituted or substituted aralkyl, Z is oxygen, R′ is alkyl or aralkyl, preferably phenylalkyl, R is a group of the formula (b), wherein R⁵ and R⁶, independently from each other, are selected from the group consisting of H, straight or branched alkyl, and cycloalkyl, or R⁵ and R⁶, when taken together with the N atom attached thereto form a 3 to 7-membered, Y⁶ is H or —OR⁷, R⁷ is H or acyl, k is 1, 2 or 3 and m is 1, 2 or 3. In some aspects, R⁵ and R⁶, independently from each other, are C₁₋₄ alkyl. In other aspects, R⁵ and R⁶ are taken together with the N atom attached thereto to form a 5 to 7-membered heterocyclic ring. In some aspects, R⁷ is unsubstituted or substituted alkylcarbonyl or arylcarbonyl.

In some aspects of this embodiment, A is phenylalkyl. In some aspects, R′ is phenylalkyl.

According to yet another embodiment, compounds of formula (IV) also include those wherein X is oxygen and Z is ═NH.

According to one embodiment, compounds of formula (IV) include those wherein A is selected from the group consisting of unsubstituted or substituted alkyl, cycloalkyl, and unsubstituted or substituted aralkyl, R is a group of the formula (b), wherein R⁵ and R⁶, independently from each other, are selected from the group consisting of H, straight or branched alkyl, and cycloalkyl, or R⁵ and R⁶ are taken together with the N atom attached thereto to form a 3 to 7-membered heterocyclic ring, Y⁶ is H or —OH, k is 1, 2 or 3 and m is 1, 2 or 3.

In some aspects of this embodiment, A is phenylalkyl, unsubstituted phenyl or phenyl substituted with halo, alkyl, haloalkyl, alkoxy or nitro. In other aspects, R⁵ and R⁶, independently from each other, are C₁₋₄ alkyl. In other aspects, R⁵ and R⁶ are taken together with the N atom attached thereto to form a 5 to 7-membered heterocyclic ring.

According to one embodiment, compounds of formula (IV) include those wherein A is a group of the formula (a):

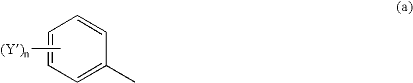

wherein Y¹ is haloalkyl, n is 1, 2 or 3, R′ is H and R is a group of the formula (b), wherein R⁵ and R⁶, independently from each other, are selected from the group consisting of H, straight or branched alkyl, and cycloalkyl, or R⁵ and R⁶ are taken together with the N atom attached thereto to form a 3 to 7-membered heterocyclic ring, Y⁶ is H or —OH, k is 1, 2 or 3 and m is 1, 2 or 3.

In some aspects of this embodiment, Y¹ is trifluoromethyl. In other aspects, R⁵ and R⁶, independently from each other, are C₁₋₄ alkyl. In other aspects, R⁵ and R⁶ are taken together with the N atom attached thereto to form a 3 to 7-membered heterocyclic ring.

According to one embodiment, compounds of formula (IV) also include the cyclic compounds of the formula (III″), wherein A is selected from the group consisting of unsubstituted phenyl, phenyl substituted with halo or nitro, and N-containing heteroaryl, R′ is H and R″ is an ω-amino-alkyl group mono- or disubstituted on the amino group, the alkyl chain of which having 1 to 5 carbon atoms and the amino substituents, independently from each other, may be one or two straight or branched alkyl or cycloalkyl, or the two amino-substituents, together with the N atom adjacent thereto, form a 3 to 7-membered, preferably 5 to 7-membered saturated heterocyclic ring, or a C₁₋₄ alkyl N-quaternary derivative thereof, with the proviso, that when A is 3-piridyl, R″ is different from 1-piperidinylmethyl.

Pharmaceutically acceptable salts of the compounds of this invention include, for example, those derived from pharmaceutically acceptable inorganic and organic acids and bases and amino acids. Examples of suitable acids include hydrochloric, hydrobromic, hydroiodidic, sulfuric, nitric, perchloric, fumaric, maleic, phosphoric, glycolic, lactic, salicylic, succinic, toluene-p-sulfonic, tartaric, acetic, citric, methanesulfonic, formic, benzoic, malonic, naphthalene-2-sulfonic and benzenesulfonic acids. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N—(C1-4 alkyl)₄⁺ salts. Salts derived from amino acids include arginine-salt, glutamic acid salt. In some embodiments, the pharmaceutically acceptable salt is derived from citric acid or maleic acid. In some embodiments, the pharmaceutically acceptable salt is derived from citric acid.

The second component of the pharmaceutical composition of the present invention is an additional therapeutic agent. Suitable additional therapeutic agents include those defined above.

The third components is a pharmaceutically acceptable carrier. Suitable pharmaceutically acceptable carriers include those defined above.

According to a preferred embodiment, the pharmaceutical composition comprises a compound of formula (III); Riluzole and a pharmaceutically acceptable carrier.

According to a preferred embodiment, the pharmaceutical composition comprises a compound of formula (IV); Riluzole and a pharmaceutically acceptable carrier.

According to another preferred embodiment, the pharmaceutical composition comprises compound (I); Riluzole and a pharmaceutically acceptable carrier.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, favoring or coloring agents may also be added.

Alternatively, the pharmaceutical compositions of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, beeswax and polyethylene glycols.

According to a preferred embodiment, the pharmaceutical compositions of this invention are orally administered.

The amount of both, the compound and the additional therapeutic agent that may be combined with the carrier materials to produce a single dosage form will vary depending upon the host treated and the particular mode of administration. Preferably, the compositions of this invention should be formulated so that a dosage of between 0.1-1 g/kg body weight/day, preferably 0.1-300 mg/kg body weight, can be administered. The dose of the compound depends on the condition and the illness of the patient, and the desired daily dose. In human therapy, the oral daily dose is preferably 10-300 mg. These doses are administered in unit dosage forms, which may be divided into 2-3 smaller doses for each day in certain cases, especially in oral treatment.

In the compositions of the present invention, the additional therapeutic agent and the compound of this invention may act synergistically. Therefore, the amount of additional therapeutic agent in such compositions will be less than that required in a monotherapy utilizing only that therapeutic agent. In such compositions a dosage of between 0.1-1 g/kg bodyweight/day of the additional therapeutic agent can be administered.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The dosage of compound will also depend upon which particular compound is in the composition.

According to another embodiment, the present invention provides method of treating a disease, disorder or condition in which molecular chaperones have been implicated. In one aspect of this embodiment, the method is used to treat a neurodegenerative disease in a patient by administering any of the pharmaceutical compositions described above. In one aspect of this embodiment, the neurodegenerative disease is selected from the group consisting of stroke, ALS, PD, AD, Huntington's Disease and cystic fibrosis. In some embodiments, the disease is ALS.

In another embodiment, the method is used to treat a neurodegenerative disease ex vivo.

In order that this invention be more fully understood, the following example are set forth. This example is for the purpose of illustration only and is not to be construed as limiting the scope of the invention in any way.

### EXAMPLES

### Example 1

### Preparation of Pharmaceutical Compositions

Pharmaceutical compositions comprising Compound I were prepared as hard gelatin capsule form suitable for oral administration. Capsule strengths were 25, 50, and 100 mg. Placebo capsules were also prepared. The content of each capsule strength is below.

| Capsules | Compound I (mg) | MC cellulose (mg) | Talc (mg) |
|---|---|---|---|
| Placebo | 0 | 277.0 | 3.0 |
| 25 mg | 25.0 | 252.0 | 3.0 |
| 50 mg | 50.0 | 227.0 | 3.0 |
| 100 mg | 100.0 | 187.0 | 3.0 |

The capsules were subjected to stability testing according to the ICH guidelines: they were stored in a qualified climatic chamber at a temperature of 40±2° C. and a relative humidity (RH) of 75±5% for six months. The capsules were stable under these conditions.

### Example 2

### Methods of Treating ALS In Mice

Transgenic mice over-expressing human mutant SOD1 have a phenotype and pathology that are very similar to that seen in human ALS patients. In this study, Compound I was tested for the ability to prevent the progressive loss of motor neurons and muscle function known to occur in mSOD1^{(G93A)} mice. mSOD1^{(G93A)} mice of both sexes were treated daily with Compound I (10 mg/kg, ip) from 35 or 70 days of age. Part of the data is reported below, and is reported in Kieran D, Kalmar B, Dick J R, Riddoch-Contreras J, Burnstock G, Greensmith, L: Treatment with arimoclomol, a coinducer of heat shock proteins, delays disease progression in ALS mice. Nat. Med. 10(4), p. 345-7 (2004), which is incorporated by reference.

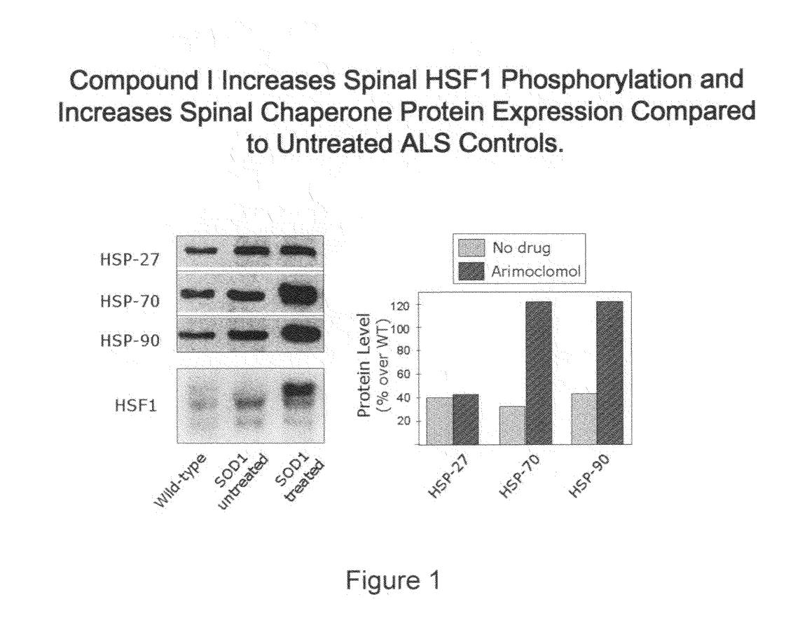

The results from this experiment are as follows. The activation of HSF-1, as well as the expression of Hsp70 and Hsp90, was examined in the spinal cord of experimental mice. We found that HSF-1 was present in the spinal cord of wild-type, untreated and Compound I-treated mSOD1^{(G93A)} mice. However, in Compound I-treated mSOD1^{(G93A)} mice, Western blot analysis revealed an observable ‘band shift’ of HSF-1, indicative of a stress-induced activation of HSF-1 by hyper-phosphorylation. Immunostaining revealed that, at 120 days of age, expression of Hsp70 and Hsp90 was increased in the lumbar spinal cords of both untreated and Compound 1-treated SOD1^{(G93A)} mice, although there was a clear increase in the intensity of Hsp70 and Hsp90 immunoreactivity in motor neurons of Compound I-treated mSOD1^{(G93A)} mice (FIG. 1). Immunostaining for human SOD1 confirmed that the human mutant protein was indeed present in motor neurons of both treated and untreated mice although no immunoreactivity was observed in spinal cords from wild type mice.

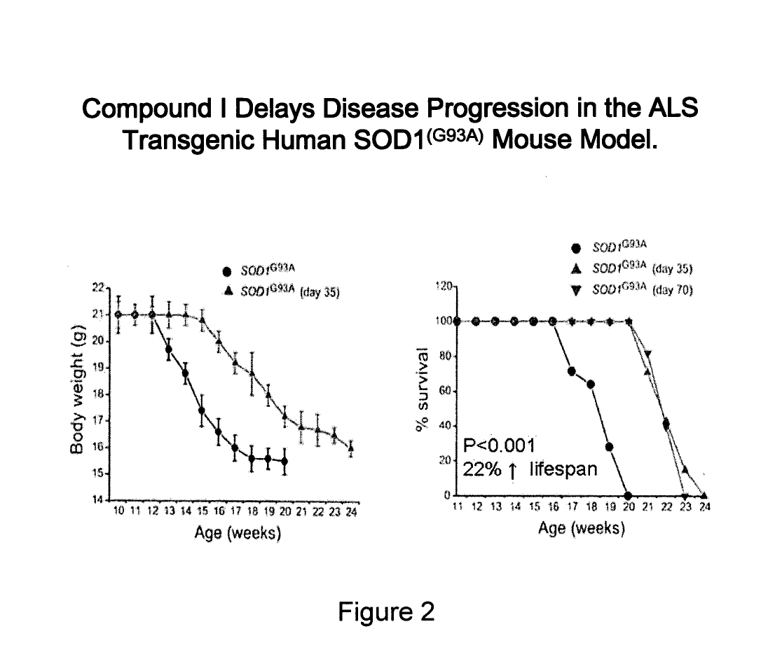

Untreated mSOD1^{(G93A)} mice had a average lifespan of 125 days (±1.8 SEM, n=18), as determined by both an inability of the mouse to right itself when put on its side as well as a 20% reduction in body weight. However, in the Compound I-treated group, the decline in body weight was delayed and lifespan was significantly improved (FIG. 2). Thus, Compound I-treated mice lived an average of 153 days (±2.6 SEM, n=7). This represents a significant increase in lifespan of over 22% (p=<0.001). The effect of beginning Compound I treatment at the time of disease onset was also tested by starting treatment at 70 days of age, when the first signs of locomotor defects are observed. Compound I treatment from 70 days of age extended the mean lifespan of mSOD1^{(G93A)} mice by 23 days, from 125 days (±1.8 SEM, n=18) in the untreated group to 148 days (±1.5 SEM, n=5) in the treated group. This represents an increase in lifespan of 18% (p=<0.001).

Interestingly, this increase in lifespan was not significantly different from the increase in lifespan observed in mSOD1^{(G93A)} mice treated from 35 days of age (p=0.3), indicating that Compound I treatment is equally beneficial, even when administered at the time of symptom onset.

In conclusion, Compound I significantly delayed the progression of disease in the ALS transgenic mouse model, apparently by increasing phosphorylation HSF-1 and the resulting increased expression of Hsp70 and Hsp90. Please note that the effect reported above indicating a highly significant improvement in lifespan has not proven to be reproducible between laboratories. While the reason for the lack of reproducibility with regard to lifespan in this model is unclear, the improvements in motor neuron and muscle viability described in the published paper are reproducible.

### Example 3

### Pharmacokinetics in Dogs

Pharmacokinetics and elimination of total radioactivity were studied in male Beagle dogs (n=6) following intravenous and oral administration of ¹⁴C-labeled Compound I at 6.45 mg/kg (equal to 4 mg/kg free base) dose level. In the same serum samples the concentrations of parent compound were also measured by HPLC.

Pharmacokinetic analysis was performed on the individual serum level versus time curves using non-compartmental analysis. Pharmacokinetic analysis of Compound I serum level curves was also carried out using compartmental models. After iv administration the two-compartmental model resulted in the best fitting. Oral serum level curves were evaluated with the one-compartmental model. Following intravenous administration, little inter-individual variability was found. In the course of HPLC analysis, the first concentration measured 5 min after dosing was 2.19±0.27 μg/ml. Compound I has a large central volume of distribution (V_{c}=1.50±0.5 l/kg) indicating very good distribution properties. Inspecting the intravenous curve two straight decreasing phases was seen. The first apparent elimination half-life was short (0.33±0.51 h). This phase is characteristic of the distribution process of Compound I. The second apparent elimination half-life was 1.26±0.20 h, which is dominantly characteristic of the elimination processes of Compound I. Around the 8^{th} post-treatment hour the levels decreased near to the quantification limit (10 ng/ml). Considering the relatively high total serum clearance, metabolic elimination of Compound I seemed to be likely.

However, the Compound I oral serum kinetic profile showed high inter-individual variability. The absorption of Compound I started between 15-120 min after oral dosing. The process proved to be fast, the absorption half-life was 0.38 h. The peak concentrations (0.94±0.37 μg/ml) occurred between 1-2 h (except the dogs SM01, 0.5 h and SM04, 4 h). In general, the serum levels decreased from the peaks to near to the quantification limit at 8 h at a constant rate. The elimination half-lives were similar to that of intravenous curves. The oral bioavailability of Compound I was 77%.

After intravenous administration similarly to the parent compound, the radioactivity in serum also showed little inter-individual variability. The first concentration measured 5 min after dosing was 2.29±0.32 μg/ml. After a short decrease of levels a plateau or slight increase of concentrations could be seen. At the same time the concentration of parent compound decreased systematically. The unusual behavior of radioactivity was probably due to the metabolic transformation of Compound I resulting in metabolites having a smaller volume of distribution. A continuous decrease of the serum levels was noticeable during the two weeks. The terminal elimination half-life of radioactivity was much longer than that of Compound I. The mean residence time of total radioactivity was significantly longer compared to the parent compound and consequently was the clearance, where the total radioactivity was smaller than in the case of Compound I. The AUC values for radioactivity were about 8 times higher than those of the parent compound. The facts mentioned above indicate that intense metabolic transformation of Compound I took place resulting in metabolite(s) having a longer elimination half-life and probably a different volume of distribution.

After oral dosing the peaks of radioactivity could be detected later than those of the parent compound. The peak concentrations of radioactivity were 1.7 times higher than those of Compound I. From the peaks until 12 h the serum radioactivity levels decreased at a constant rate. At 12 h the concentrations were about 15% of the peaks. The terminal elimination half-life was similar to that following intravenous dosing and was also much longer than the corresponding value of the parent compound. The radioactivity concentrations were measurable for two weeks (0.014±0.004 μgE/ml). The mean AUC of individuals was about 10 times higher than that of the parent compound. The oral bioavailability of total radioactivity proved to be 104% indicating the perfect absorption of Compound I from the gastrointestinal tract.

During the 7 day collecting period the total recoveries were near complete. The major part of the compound and its metabolites was eliminated with urine. The elimination was rapid, the half of total radioactivity was excreted during the first 12 h. The total recoveries in feces samples were lower. Biliary excretion proved to be a minor elimination route.

In good agreement with the result of serum level studies, the large amount of radioactivity in urine and the similar rate and extent of elimination after both intravenous and oral ways of treatment indicated perfect oral absorption.

### Example 4

### Pharmacokinetics in Rats

The pharmacokinetics of unlabeled Compound I were studied in rats after a single intravenous and oral doses of 25.8 mg/kg (equal to 16 mg/kg free base) in male and female animals (Study A: Compound I/PRE SK-005). Each (male and female) group consisted of 8 animals and blood samples were taken from four animals at each time point from the sublingual vein. After oral administration the serum level curves did not show differences between males and females, but after intravenous dosing the terminal half life was different in male and female rats.

The pharmacokinetic properties of total radioactivity were studied in male and female Wistar rats at the dose of 25.8 mg/kg ¹⁴C labeled Compound I (equal to 16 mg/kg free base) administered intravenously and orally (Study B: Compound I/PRE SK-007). The concentrations of total radioactivity were monitored by liquid scintillation counting and whole-body autoradiographic methods in serum, organs, tissues, and excreta. All parts of the study were performed in both sexes (n=5 in both sexes).

After intravenous administration the serum level curves did not show differences between males and females, but after oral dosing both the serum and the organ levels indicated marked sex-dependent differences. Pharmacokinetic analysis was performed on the basis of the mean serum levels of parent compounds as well as on individual serum levels of radioactivity versus time curves using non-compartmental analysis.

Study A: Compound I might be characterized as having rapid absorption and distribution and slightly slower, but still fast, elimination. It was detectable in the serum in a relatively high concentration 10 min after the oral treatment and reached the peak values in the 15-minute samples in both male and female animals. The serum level decreased significantly from the second hour and it was below the quantification limit (0.01 μg/ml) from the 8^{th} hour.

The Compound I level decreased to 4% and 10% of the appropriate initial concentrations in male and female animals, respectively, two hours after the intravenous treatment. The concentrations were below the quantification limit (0.01 μg/ml) from the 6^{th} hour in male animals and from the 12^{th} hour in female rats.

The values of MRT (Mean Residence Time) suggest that the absorption and the elimination of the drug were fast.

The clearance of Compound I was higher than the hepatic clearance suggesting that the drug is eliminated not only by the liver but also by other organs (possibly by the kidneys). The bioavailability of the drug was good, 78% for the male, 90% for the female group. These data suggest that the role of the first pass effect in the elimination of Compound I is less important than other routes, possibly the excretion via the kidneys.

In conclusion, Compound I showed a biphasic kinetics, with a rapid distribution and a slower elimination phase. The clearance of the drug was high, higher than the hepatic plasma flow, suggesting that the drug is eliminated not only by the liver but also by the kidneys. The elimination and absorption were also fast. The bioavailability of the drug was 78 and 90%, respectively (in the male and female groups).

Study B: After single intravenous dosing the distribution and/or elimination of radioactivity from the serum was very rapid resulting in fast first decrease of radioactivity levels. By the 3^{rd} post-treatment hour the concentrations decreased to 20% of the first measured concentration (about 10 μgE/ml at 5 min). From this time the levels hardly decreased or increased indicating enterohepatic recirculation. From 24 h post-dose the radioactivity eliminated very slowly. The Mean Residence Time was long and the total clearance was small.

After oral administration the absorption of radioactivity was started immediately. The first maximal serum concentrations occurred within 1 h and were about two times higher in females than those in males.

After the first peak, further peak(s) could be detected and the elimination of the residual radioactivity was as slow as that after intravenous administration.

After both intravenous and oral administration most of the administered radioactivity eliminated rapidly. The total recovery in urine and feces was about 80% during 48 h. At the same time, the remaining part of the radioactivity was eliminated very slowly. At the end of the one-week period almost 20% of the dose remained in the rats.

The drug related materials were eliminated in both urine and feces approximately in the same ratio. The same elimination pattern after intravenous and oral administration indicated the good absorption of radioactivity from the gastro-intestinal tract.

A significant part of the administered dose was eliminated with bile (20-30% of dose) during 24 h. The intense elimination with bile would make enterohepatic recirculation possible.

Organ levels of total radioactivity were measured at the time of the first serum peak (45 min after single oral administration). Most of the radioactivity in blood was localized in serum fraction. In good correlation with the serum concentrations the organ radioactivity levels were about 2-times higher in females than those in males. The levels in almost all organs and tissues were higher than the actual serum concentration indicating good distribution properties of Compound I. The low radioactivity in the brain indicated weak penetration through the blood-brain barrier. The highest tissue concentrations were measured in liver and kidney due to the intense elimination processes.

At the 4^{th} post-treatment hour, the radioactivity concentrations were approximately 20-60% of the 45 min values. However, while the differences decreased, the concentrations in general remained a little higher in females than in males. The only exceptions were in the blood, serum and the excretory organs.

Contrary to the earlier time-point, the radioactivity levels were similar or lower in most organs than the actual serum concentration. The metabolite(s) may have smaller volume(s) of distribution than that of the parent compound.

By the 24^{th} post-treatment hour, in general, further decrease of radioactivity levels could be observed. Relatively high levels remained in the liver, kidney and adrenals. The concentrations increased slightly in brown fat and Harderian glands. Although the absolute concentration was low in the brain, the levels remained the same as at 4 h. In contrast to the results at the earlier time-points, at 24 h post-dose the distribution in blood was changed indicating strong binding of the residual radioactivity to the red blood cells.

In pregnant females, the distribution of radioactivity showed only slight differences compared to that in non-pregnant females. The radioactivity penetrated into the fetuses, but considering the radioactivity found in fetal membrane and placenta, they had barrier function. At the early time-point the concentrations in the fetuses were smaller than their mother's actual serum levels. By 24 h the organ levels in the fetus as became similar to those of the mothers. It should be emphasized that the brain levels in fetuses were low but higher than the corresponding brain levels of the mothers at all of the three investigated time-points.

Once daily multiple oral administrations of ¹⁴C labeled Compound I were carried out in male and female Wistar rats, at the dose of 25.8 mg/kg (equal to 16 mg/kg free base) for 7 days (Study B: Compound I/PRE SK-007). They resulted in marked accumulation of radioactivity in serum. The pre-dose concentrations in the 7^{th} day approximated the peak levels after single treatment; the total exposure during the last day (0-24 h AUD) was more than two times higher than that in the first day.

During the multiple administration, the radioactivity accumulated not only in the serum but also in all organs and tissues. The ratios of increase were very different (4- to 12-fold) in the different organs and tissues.

### Example 5

### Toxicokinetics in Rats

A 28-day repeated dose oral toxicokinetic study of Compound I was performed in rats involving dose groups of 375 mg/kg, 750 mg/kg and 1500 mg/kg/day (equal to 233 mg/kg, 465 mg/kg and 930 mg/kg free base) (n=5 animals/sex/group). Blood samples were taken after the first and after the last doses to determine the kinetic parameters.

The serum concentration of Compound I increased during the first hour (375 mg/kg) or during the second hour (750 and 1500 mg/kg) both in male and female rats after the first treatment. A rapid decrease was observed from the 4^{th} hour (375 mg/kg) and 8^{th} hour (750 and 1500 mg/kg).

Terminally, the peak concentrations were found 0.5 h (375 mg/kg male and female, 750 mg/kg male), 1 h (750 mg/kg and 1500 mg/kg female) and 2 h (1500 mg/kg male) after the treatment. The serum levels decreased rapidly from the 2^{nd} (375 mg/kg), 4^{th} (750 mg/kg) and 8^{th} hour (1500 mg/kg) both in male and female animals.

The peak concentrations of Compound I increased, but not straightly, with the increasing doses after the first and 28^{th} day treatments in female animals and the differences between the peak concentrations were slightly less at termination of the study than at the first treatment.

The AUCₜₒₜ increased with the doses, but not linearly. This might mean that the first pass metabolism of the drug decreased due to the high doses. After the 28^{th} dose, the AUC values were below the expected value which was measured at the first treatment, suggesting that the metabolism of the drug increased and the bioavailability decreased. This phenomenon may result from increased first pass metabolism, or from decreased absorption. It is possible that Compound I induced some metabolic enzymes and in this way its own elimination.

The MRT became longer after the first 1500 mg/kg dose, but hardly shows any dose dependency after the 28^{th} dose. This also suggests that some inductive effect may be in the background. t_{1/2} runs parallel with MRT.

The clearance was high after every dose, it increased with time and was not influenced by the dose, showing that the elimination capacity of the rats is very high. Summary data are recorded in Tables 1 (Day 1 treatment) and 2 (Day 28 treatment).

TABLE 1
Summary of rat pharmacokinetic data - Day 1.

| | Cmax | | AUCₜₒₜ | | | Clear- | |
|---|---|---|---|---|---|---|---|
| | (mg/ | Tmax | (h * | t_{1/2} | MRT | ance | Vz |
| | L) | (h) | mg/L) | (h) | (h) | (L/h/kg) | (L/kg) |
| Male | | | | | | | |
| 375 mg/kg | 17.64 | 1 | 48.39 | 3.09 | 2.60 | 4.78 | 21.34 |
| 750 mg/kg | 43.48 | 2 | 143.51 | 2.63 | 3.00 | 3.23 | 12.22 |
| 1500 mg/kg | 31.86 | 2 | 222.46 | 19.20 | 17.22 | 4.16 | 115.24 |
| Female | | | | | | | |
| 375 mg/kg | 19.96 | 1 | 47.94 | 5.55 | 2.37 | 4.83 | 38.61 |
| 750 mg/kg | 29.15 | 2 | 154.96 | 2.28 | 4.08 | 2.99 | 9.83 |
| 1500 mg/kg | 38.57 | 2 | 278.19 | 7.59 | 9.88 | 3.33 | 36.44 |
| Male + Female | | | | | | | |
| 375 mg/kg | 18.8 | 1 | 48.16 | 3.86 | 2.46 | 4.80 | 26.76 |
| 750 mg/kg | 36.32 | 2 | 149.33 | 2.27 | 3.56 | 3.10 | 10.16 |
| 1500 mg/kg | 34.84 | 2 | 247.04 | 9.81 | 11.07 | 3.75 | 53.01 |

Mean values were calculated by software KINETICA ™.

TABLE 2
Summary of rat pharmacokinetic data - Day 28.

| | Cmax | | AUCₜₒₜ | | | Clear- | |
|---|---|---|---|---|---|---|---|
| | (mg/ | Tmax | (h * | t_{1/2} | MRT | ance | Vz |
| | L) | (h) | mg/L) | (h) | (h) | (L/h/kg) | (L/kg) |
| Male | | | | | | | |
| 375 mg/kg | 12.82 | 0.5 | 28.03 | 2.64 | 1.99 | 8.26 | 31.42 |
| 750 mg/kg | 20.41 | 0.5 | 72.28 | 2.12 | 2.77 | 6.40 | 19.54 |
| 1500 mg/kg | 18.42 | 2 | 132.01 | 3.46 | 5.93 | 7.01 | 35.01 |
| Female | | | | | | | |
| 375 mg/kg | 21.18 | 0.5 | 47.35 | 2.19 | 2.63 | 4.89 | 15.42 |
| 750 mg/kg | 25.84 | 1 | 81.77 | 3.07 | 3.17 | 5.66 | 25.09 |
| 1500 mg/kg | 31.73 | 1 | 139.32 | 2.75 | 4.20 | 6.64 | 26.38 |
| Male + Female | | | | | | | |
| 375 mg/kg | 17 | 0.5 | 37.87 | 2.42 | 2.41 | 6.11 | 21.37 |
| 750 mg/kg | 22.24 | 1 | 77.06 | 2.70 | 2.96 | 6.01 | 23.44 |
| 1500 mg/kg | 22.48 | 2 | 135.95 | 3.29 | 5.04 | 6.81 | 32.34 |

Mean values were calculated by software KINETICA ™.

In conclusion, the systemic exposition of the animals increases with the doses. This increase was not linear because either the absorption decreased or more probably the elimination increased, possibly due to enzyme induction. The exposition decreased slightly after the repeated treatment in comparison with the starting value at each dose level. There were no differences in the kinetic parameters between the male and female animals.

### Example 6

### 28-day Repeated Dose Oral Toxicokinetic Study In Dogs

A 28-day repeated dose oral toxicokinetic study of Compound I was performed in dogs involving four dose groups of 70, 130, 190 and 210 mg/kg/day (equal to 43.4 mg/kg, 80.6 mg/kg, 117.8 mg/kg and 130.2 mg/kg free base) (n=4 animal/sex/group). The aim of the study was the evaluation of the toxicokinetic characteristics of the test item Compound I.

The test item was applied once daily (on a 7 days/week basis) in gelatin capsule. Blood samples were collected from all animals/sex/dose for serum analysis on the first treatment day and on the last (28^{th}) day before and after the treatment. Serum profiles of males and females were compared. Furthermore, the effect of dose escalation and the multiple dosing was also investigated. Summary data are recorded in Tables 3 (Day 1 treatment) and 4 (Day 28 treatment).

TABLE 3
Summary of dog pharmacokinetic data - Day 1.
Compound I Dog (Day 1)
M + F

| | | | AUCₜₒₜ | | | | |
|---|---|---|---|---|---|---|---|
| | Cmax | Tmax | (mg/ | t_{1/2} | MRT | Clearance | Vz |
| Dosage | (mg/L) | (h) | L × h) | (h) | (h) | (L/h/kg) | (L/kg) |
| 70 mg/kg | | | | | | | |
| Mean | 20.32 | 1.38 | 70.59 | 2.06 | 4.01 | 0.62 | 1.87 |
| SD | 3.50 | 0.22 | 11.57 | 0.54 | 0.29 | 0.10 | 0.72 |
| 130 mg/kg | | | | | | | |
| Mean | 37.97 | 2.50 | 162.14 | 1.98 | 4.61 | 0.50 | 1.43 |
| SD | 10.49 | 0.93 | 17.68 | 0.21 | 0.76 | 0.06 | 0.20 |
| 190 mg/kg | | | | | | | |
| Mean | 56.47 | 1.75 | 222.20 | 2.09 | 4.23 | 0.67 | 2.45 |
| SD | 21.85 | 0.77 | 90.03 | 0.63 | 1.30 | 0.48 | 2.00 |
| 210 mg/kg | | | | | | | |
| Mean | 64.47 | 2.31 | 255.46 | 2.06 | 4.58 | 0.52 | 1.52 |
| SD | 18.70 | 1.07 | 31.02 | 0.20 | 0.58 | 0.06 | 0.18 |

TABLE 4
Summary of dog pharmacokinetic data - Day 28.
Compound I Dog (Day 28)
M + F

| | | | AUCₜₒₜ | | | | |
|---|---|---|---|---|---|---|---|
| | Cmax | Tmax | (mg/ | t_{1/2} | MRT | Clearance | Vz |
| Dosage | (mg/L) | (h) | L × h) | (h) | (h) | (L/h/kg) | (L/kg) |
| 70 mg/kg | | | | | | | |
| Mean | 21.24 | 1.56 | 76.26 | 2.21 | 3.95 | 0.57 | 1.84 |
| SD | 2.28 | 0.45 | 4.98 | 0.10 | 0.35 | 0.04 | 0.16 |
| 130 mg/kg | | | | | | | |
| Mean | 39.09 | 1.69 | 166.31 | 2.03 | 4.23 | 0.49 | 1.44 |
| SD | 6.40 | 0.37 | 24.22 | 0.29 | 0.60 | 0.06 | 0.27 |
| 190 mg/kg | | | | | | | |
| Mean | 39.98 | 2.44 | 172.55 | 2.22 | 4.63 | 0.78 | 2.56 |
| SD | 15.95 | 0.94 | 65.48 | 0.68 | 1.29 | 0.31 | 1.15 |
| 210 mg/kg | | | | | | | |
| Mean | 81.83 | 2.44 | 288.55 | 2.21 | 4.63 | 0.46 | 1.46 |
| SD | 29.97 | 1.29 | 35.76 | 0.37 | 0.87 | 0.06 | 0.33 |

In conclusion, Compound I showed straight kinetics in dogs within the dose range of 70-210 mg/kg. The Cₘₐₓ and AUCₜₒₜ values were dose dependent, the clearance, t_{1/2} and MRT remained constant. Male and female animals showed the same kinetic properties.

A 110-day dog oral multi-dose toxicokinetic study was also run, and these results are depicted in Table 4a. Drug exposure was dose linear and did not change with repeat dosing.

TABLE 4a
110 Day Dog Oral Multi-Dose Toxicokinetics

| | Cmax | | AUCtot | t½ | MRT | Clearance |
|---|---|---|---|---|---|---|
| Dosage | (mg/L) | Tmax (h) | (mg/L × h) | (h) | (h) | (L/h/kg) |
| 50 mg/kg | | | | | | |
| Mean | 10.84 | 1.78 | 40.22 | 1.89 | 3.78 | 0.78 |
| SD | 1.33 | 1.01 | 7.80 | 0.09 | 0.44 | 0.16 |
| 80 mg/kg | | | | | | |
| Mean | 22.00 | 1.88 | 87.01 | 2.10 | 4.04 | 0.57 |
| SD | 4.78 | 0.69 | 10.12 | 0.56 | 0.48 | 0.07 |
| 160 mg/kg | | | | | | |
| Mean | 37.95 | 1.94 | 166.39 | 2.44 | 4.57 | 0.62 |
| SD | 9.51 | 0.81 | 33.54 | 0.44 | 0.57 | 0.12 |

Relative animal toxicokinetic and human pharmacokinetic drug exposures are summarized in Table 4b. These data indicate that pharmacologic exposures greater than the minimum effective dose in animals can safely be achieved in humans.

TABLE 4B
Relative Animal Toxicokinetic and Human Pharmacokinetic Drug
Exposures

| | | | | | Fold- |
|---|---|---|---|---|---|
| | | | Fold- | | above |
| | Maximum | Fold-below | below | Fold-above | minimum |
| | human drug | dog drug | rat drug | minimum | effective |
| | exposure | exposure at | exposure | effective rat | mouse |
| | (100 mg tid) | NOAEL | at NOAEL | exposure | exposure |
| AUC | (1.0) | 17 | 6.0 | 5.9 | 12 |
| (ug × | | | | | |
| h/ml) | | | | | |
| Cmax | (1.0) | 5 | 28 | 1.4 | 2.8 |
| (ug/ | | | | | |
| ml) | | | | | |

### Example 7

### In Vitro Metabolism

Metabolic studies in primary hepatocytes were performed to estimate the rate of metabolism and to demonstrate the similarities and differences in biotransformation of Compound I by comparing catalytic activities of rat, dog and human hepatocytes. Different concentrations of Compound I (10, 20 and 40 μM) were added to the hepatocyte preparation directly. The samples were examined in 0, 5, 15, 30, 60, and 240 min.

Rat hepatocytes were the most active in biotransformation of Compound I; the rate of Compound I metabolism was much slower in dog liver cells and human hepatocytes were the least active. During the metabolism of ¹⁴C-Compound I, rat hepatocytes produced four metabolites (Nos. 1-4). Two of them could be considered as the main metabolites (No. 3: 47.7%; No. 4: 15.1% of the total radioactivity) and two more were minor products (No. 1: 0.32%; No. 2: 1.89%).

Two metabolites were detected in the extract of the incubation media of dog liver cells. The amounts of No. 3 (4.9% of the total radioactivity) and No. 4 (3.3%) were less than those produced by rat hepatocytes. Dog cells did not form metabolites No. 1 and No. 2.

Although human liver cells were not so active in biotransformation of Compound I as rat cells, five metabolites were produced during the 4 hour incubation period. Four of them (No. 1-4) were found to be identical with the metabolites produced by rat hepatocytes, but there was one more (No. 5) formed by human hepatocytes that was not detected among the metabolites produced by rat or dog cells. The amounts of the metabolite No. 1, No. 2, No. 3 and No. 4 were quite small (0.64%, 0.60%, 0.80% and 0.62%, respectively), while the amount of No. 5 was 2.59% of the total radioactivity. Subsequent formula determination studies concluded that this human specific metabolite is bimoclomol, whose safety has been demonstrated in several clinical studies.

The kinetic parameters of Compound I in 10 μM concentration: t_{1/2} was 0.82, 12.6 and 17.2 hour and the clearance was 0.84, 0.055 and 0.040 ml/hour in rats, dogs and human samples, respectively.

### Example 8

### Acute Oral Toxicology in Rats and Mice

All animal toxicology studies were performed at Toxicological Research Centre (Veszprém, Hungary) using protocols compliant with OECD guidelines and Principles of Good Laboratory Practice. Following is a summary of the studies conducted.

Acute oral toxicity studies were performed in rats (CRL(WI:)BR) and in mice (CRL: NMRI BR). A control and five dose groups were involved in the rat study at dose levels of 2000 mg/kg, 2600 mg/kg, 3400 mg/kg, 4400 mg/kg and 5700 mg/kg (equal to 1241 mg/kg, 1613 mg/kg, 2180 mg/kg, 2729 mg/kg and 3535 mg/kg free base, n=5 animals/sex/group). A control and one dose group were involved in the mouse study at dose level 5000 mg/kg (equal to 3101 mg/kg free base, n=5 animals/sex/group).

In the case of rats the following clinical signs due to the test item effect were observed in all dose groups: decreased activity, tremor, convulsions, ventral position, decreased righting reflex, decreased grip and limb tone, decreased body tone, squatting position, piloerection, dyspnea. Tremor and convulsions occurred in several cases as a provoked reaction to different stimuli and those were localized on the head or on the upper part of the body. The first symptoms were observed in most cases one hour after the treatment. Surviving animals became symptom-free between one and three days thereafter.

In mice the test item Compound I caused decreased activity, tremor, ventral position, squatting position, in-coordination, decreased righting reflex, decreased grip and limb tone, decreased body tone, piloerection and dyspnea. The first symptoms appeared 5-12 min after the application. Most of the animals became symptom-free on the first day. Two animals became normal on the second day.

Slight lowering of body weight gain was found in the male surviving rats on the first week and terminally.

The test item did not influence the mean body weight and body weight gain of mice during the study.

In the dead rats severe focal necrosis, vacuolar degeneration and hemorrhages were found in the liver, hyperemia and acute tubulonephrosis were observed in the kidney, erosion, hemorrhages and acute catarrh were noticed in the stomach and were related to the high dose of the test item.

In the surviving rats slight vacuolar degeneration in the liver, slight acute tubulonephrosis in the kidneys and slight catarrh in the stomach were present in a lesser degree and those were related to the high dose of the test item (male rats at dose levels of 2000 and 2600 mg/kg, male and female rats at dose levels of 3400 mg/kg).

In mice no macroscopic alteration related to the toxic effect of the test item was found during the necropsy.

In conclusion, the acute oral LD₅₀ values of test item Compound I in CRL:(WI)BR rats and CRL:NMRI BR mice are presented in Table 5.

TABLE 5
The acute oral LD₅₀ values.

Oral_{LD50} value (mg/kg)

| Species | Male | Female |
|---|---|---|
| RAT | 2828 (2297-3460) - by citrate | 3188 - by citrate |
| | 1754 (1425-2146) - by base | 1977 - by base |
| MOUSE | >5000 - by citrate | >5000 - by citrate |
| | >3101 - by base | >3101 - by base |

### Example 9

### Acute Intraperitoneal Toxicology in Rats and Mice

Acute intraperitoneal toxicity studies were performed in rats (CRL(WI:)BR) and in mice (CRL: NMRI BR).

A control and five dose groups were involved in the rat study at dose levels of 500 mg/kg, 650 mg/kg, 800 mg/kg, 950 mg/kg and 1200 mg/kg (equal to 310 mg/kg, 403 mg/kg, 496 mg/kg, 589 mg/kg and 744 mg/kg free base, n=5 animals/sex/group).

In the rat study no clinical symptoms appeared in the control group at the dose levels of 500 mg/kg (equal to 310 mg/kg free base in male) and 650 mg/kg (equal to 403 mg/kg free base in female). In the higher dose groups, the reactions of the rats due to the test item effect were: decreased activity, tremor, convulsion, squatting position, ventral position, decreased righting reflex, decreased grip and limb tone, decreased body tone, in-coordination and dyspnea. The first symptoms were observed between 30-60 min after the treatment and deaths occurred between 40 min-5 h after the treatment. The surviving animals became symptom free on the first day.

In the case of the mouse study a control and five dose groups were involved at dose levels of 850 mg/kg, 1100 mg/kg, 1500 mg/kg, 1900 mg/kg and 2600 mg/kg (equal to 527 mg/kg, 682 mg/kg, 930 mg/kg, 1178 mg/kg and 1613 mg/kg free base, n=5 animals/sex/group).

The following clinical symptoms were observed in mice due to the test item: decreased activity, tremor, convulsion, squatting position, ventral position, decreased righting reflex, decreased grip and limb tone, decreased body tone, dyspnea and piloerection. The first symptoms appeared 3-15 minutes after the application and deaths occurred between 5 and 60 min after the treatment. All surviving animals became normal on the first day.

The body weight changed in the same manner in the control and dose groups both in rats and mice.

The test item did not cause macroscopic alterations in the examined organs of surviving rats and mice. In the dead rats where a test item related effect cannot be excluded, pale and nutmeg-like patterned liver was found in a relative high frequency.

In conclusion, the acute intraperitoneal LD₅₀ values of test item Compound I in CRL:(WI)BR rats and CRL:NMRI BR mice are presented in Table 6.

TABLE 6
Acute intraperitoneal LD₅₀ values.

Intraperitoneal LD₅₀ value (mg/kg)

| Species | Male | Female |
|---|---|---|
| RAT | 1125 (931-6241) by citrate | 1241 - by citrate |
| | 698 (577-3871) by base | 770 - by base |
| MOUSE | 1352 (1087-1650 by citrate | 1189 - by citrate |
| | 839 (674-1023) by base | 738 - by base |

### Example 10

### Acute Intravenous Toxicology in Rats and Mice

A control and five dose groups were involved in the rat study at dose levels of 150 mg/kg, 200 mg/kg, 250 mg/kg, 300 mg/kg and 350 mg/kg (equal to 93 mg/kg, 124 mg/kg, 155 mg/kg, 186 mg/kg and 217 mg/kg free base, n=5 animals/sex/group).

A control and five dose groups were involved in the mouse study, at dose levels of 200 mg/kg, 250 mg/kg, 300 mg/kg, 360 mg/kg and 450 mg/kg (equal to 142 mg/kg, 155 mg/kg, 186 mg/kg, 223 mg/kg and 279 mg/kg free base, n=5 animals/sex/group).

Clinical signs of rats and mice due to the test item effect were: decreased activity, vocalization, tremor, convulsions, ventral position, squatting position, dyspnea and piloerection. Symptoms appeared immediately after or during the treatment and those animals that died did so 10-120 s after the treatment. Duration of symptoms was between 5 and 12 min.

No significant differences from the control were found in the body weight and body weight gain during either the rat or the mouse study.

No macroscopic alterations referred to the toxic effect of the test item were found in the surviving and dead rats and mice at the necropsy.

TABLE 7
The acute intravenous LD₅₀ values.

Intravenous_{LD50} value (mg/kg)

| Species | Male | Female |
|---|---|---|
| RAT | 317 (271-485) by citrate | 346 (306-1232) by citrate |
| | 197 (168-301) by base | 215 (190-764) by base |
| MOUSE | 307 (265-359) by citrate | 254 - by citrate |
| | 190 (164-223) by base | 158 - by base |

In conclusion, the acute intravenous LD₅₀ values of test item Compound in CRL:(WI)BR rats and CRL:NMRI BR mice are presented in Table 7.

### Example 11

### Repeated Dose Toxicity in Dogs

A 14-day repeated dose oral toxicity study of Compound I was performed in Beagle dogs involving control and 210 mg/kg/day dose groups (equal to 130 mg/kg free base) in both sexes (n=3 animals/sex/group). The test item was applied once daily (on a 7 days/week basis) by oral application, in gelatin capsule. The control animals were treated in the same way with placebo gelatin capsules.

The checks of mortality were done twice daily. The clinical observations were performed at least twice daily and the weighing of non-consumed food was done daily. The body weights of the animals were measured once a week. The hematological, clinical chemical and ECG investigations, urinalysis and the opthalmological examinations were performed prior to the treatment and at the end of the treatment period. Terminally gross necropsy and histopathological examinations were performed according to the study plan.

The following seven results were obtained. First, no death occurred during the study. Signs due to the test item Compound I in the 210 mg/kg dose group were decreased activity, tremor, ptosis, salivation, increased muscle tone, vomiting and thin feces. Considering the above we can state that, among other systems, the nervous system proved to be one point of attack of the test item as manifested in neurological clinical signs. Second, the treatment did not influence the mean body weight gain and the average food consumption of the animals. Third, no direct treatment related ECG or opthalmological alterations were found. Fourth, the applied dose levels of Compound I did not cause any changes of the parameters of the hematology, clinical chemistry and urinalysis examinations during the 14-day oral administration which could refer to the injury of any organs of vital importance. Fifth, macroscopic alteration in connection with the toxic effect of the test item could not be found. Sixth, in the important increase in the liver organ weight observed in the male animals the effect of 210 mg/kg Compound I cannot be excluded. Seventh, the test item caused moderate degree proliferation of cells belonging to the mononuclear phagocyte system (MPS) of the liver, slight decrease of glycogen content of hepatocytes in the male and female animals and moderate vacuolar degeneration in the liver of female animals. In addition it cannot be excluded that the 210 mg/kg dose of test item played a role in a neurohormonal disorder, affecting the cyclic function of the ovaries of the female animals.

### Example 12

### 28-Day Repeated Toxicity in Rats

28-day repeated dose oral toxicity study of Compound I was performed in rats involving control and dose groups of 375 mg/kg, 750 mg/kg and 1500 mg/kg/day (equal to 233 mg/kg, 465 mg/kg and 930 mg/kg free base) (n=5 animals/sex/group). To assess recovery post treatment, an additional 5 males and females were included in both the control and highest dosage groups. Treatment was carried out by gavage once daily. Control animals were treated with physiologic saline in the same way.

Pre-treatment: general condition and behavior activity patterns were observed. Also in a subgroup of 5 male and 5 female animals, hematological and clinical chemistry tests were performed.

During the treatment period and following a two-week recovery period the clinical observations were made daily. At weekly intervals body weight and food consumption were measured.

At the end of the treatment, hematological, clinical chemistry evaluation and gross necropsy with organ weight assessment were conducted. Full histopathology was performed on the preserved organs and tissues of the control and high dose groups. In addition, the liver and kidneys and the organs showing macroscopic changes were examined microscopically in the low and middle dose groups, too.

The following results were obtained. No deaths occurred during the study. Decreased activity, tremor, squatting position, piloerection and dyspnea were observed at the dose level of 1500 mg/kg between days 1 and 13. Tremor was localized on the head and on the upper part of the body.

Reversible body weight gain and food consumption depression due to the test item effect was found in the male animals at the dose level of 1500 mg/kg.

Dose dependent slight decrease in the RBC count was observed which was connected with the decrease in the HTC value and HGB concentration in female animals.

Dose dependent increase in the cholesterol levels (750 mg/kg and 1500 mg/kg male, 375 mg/kg, 750 mg/kg and 1500 mg/kg female) and total bilirubin concentration (male: 750 mg/kg and 1500 mg/kg and female: 1500 mg/kg) was assessed as a test item-related effect.

A test-item induced liver weight increase was observed in male and female animals at the dose levels of 750 mg/kg and 1500 mg/kg. This alteration was not completely reversible at the end of the recovery period at the dose level of 1500 mg/kg.

A dose dependent decrease of the thymus weight was found terminally both in male and female groups. The influence of the test item cannot be excluded and verified on the basis of the results of the study.

Macroscopic and microscopic alterations related to the toxic effect of the test item were not found.

The histometrical examination of the lymphoid organs revealed neither hyperplasic nor regressive alterations in any dose groups, referring to an immunostimulating or immunosuppressive effect of the test item.

The bone marrow smear evaluation did not reveal any differences between the control and dose group of 1500 mg/kg in the hemopoiesis.

In conclusion, based upon these findings in male and female CRL(:WI)BR rats the dose of 375 mg/kg/day by citrate (233 mg/kg/day by base) is the “No observed adverse effect level” (NOAEL).

### Example 13

### 28-Day Repeated Dose Oral Toxicity in Dogs

28-day repeated dose oral toxicity study of Compound I was performed in dogs involving control and four dose groups of 70, 130, 190 and 210 mg/kg/day (equal to 43.4 mg/kg, 80.6 mg/kg 117.8 mg/kg and 130.2 mg/kg free base) (n=4 animals/sex/group). The test item was applied once daily (on a 7 days/week basis) by oral application, in gelatin capsules. The control animals were treated in the same manner with placebo gelatin capsules.

Pre-treatment: hematological, clinical chemistry and ECG investigations and opthalmoscopic examinations were performed.

During the treatment period: check of mortality and clinical observation were performed at least twice daily and weighing the non-consumed food was performed daily. The body weights of animals were measured once a week. At the end of the treatment, hematological laboratory and ECG investigations, opthalmoscopic examinations, gross necropsy, histopathological and histometrical examinations were performed.

The following results were obtained. No deaths occurred during the study. The test item Compound I caused in the 130 mg/kg, 190 mg/kg, 210 mg/kg dose groups decreased activity, tremor, ataxia, vomiting, thin and thin sanguineous feces. The frequency of these clinical signs showed dose dependency. In the 130 mg/kg dose group fear, effort-induced ataxia, tonico-clonic bodily extended twitching, weakness, abasia for a short period, tachypnea, salivation and tenesmus occurred in the 190 mg/kg dose group fear, ataxia, pelvic limb weakness occurred and in the 210 mg/kg dose group dancing-like movements, salivation and tenesmus occurred. All were treated as test item related clinical signs.

Body weight gain depression due to the test item effect was found in the 130 mg/kg, 190 mg/kg and 210 mg/kg dose groups. The appetite of the animals (female 130 mg/kg and male 210 mg/kg dose groups) was also affected.

The test item did not cause ECG and opthalmologic alterations.

The test item Compound I did not cause any severe changes of the hematological and clinical chemical parameters which could refer to the injury of any organs of vital importance. The test item had a suspected glucose-level decreasing effect in the male animals of 190 mg/kg and 210 mg/kg dose groups.

Macroscopic alteration in connection with the toxic effect of the test item Compound I could not be found.

A test item induced moderate increase of the liver weight in the male 130, 190 and 210 mg/kg dose groups occurred.

The test item showed moderate hepato-toxic effect in the 130 mg/kg, 190 mg/kg and 210 mg/kg doses causing moderate degree proliferation of cells belonging to the mononuclear phagocyte system (MPS) of the liver of all female animals in the 210 and 190 mg/kg dose groups and 2 female animals (50%) in the 130 mg/kg dose group. This alteration occurred in the liver of male animals only in the 210 mg/kg-dose group. In some of the female and male animals belonging to the higher dose groups, zonal decrease of glycogen content in the liver—without any degenerative lesions—was also detectable. No treatment related histopathological alteration occurred in the 70 mg/kg female, and in the 70, 130 and 190 mg/kg male dose groups.

The item Compound I did not influence the bone marrow function.

In the histometrical examination of the lymphoid organs neither hyperplastic nor regressive alterations could be detected in any dose groups, referring to an immunostimulating or immunosuppressive effect of the test item.

In conclusion, based upon these findings in male and female Beagle dogs the dose of 70 mg/kg/day by citrate (43.4 mg/kg/day by base) is the “No observed adverse effect level” (NOAEL).

### Example 14

### 110-Day Repeated Dose Oral Toxicity Study in Dogs

Evaluation of the toxic characteristics of the test item Compound I in a 90-day repeated dose oral toxicity study was performed in beagle dogs involving control and three dose groups of 50, 80 and 160 mg/kg/day (equal to 31.0 mg/kg, 49.6 mg/kg and 99.2 mg/kg free base).

Four animals of both sexes/groups were used, except the control and high dose group where the groups were completed with 2 recovery animals in both sexes. The test item was applied once daily (on a 7 days/week basis) by oral application, in gelatin capsules for 112 days.

The check of mortality and the clinical observations were performed twice daily, the weighing of non-consumed food was done daily. The body weight of the animals was measured once a week. The urine, hematological, clinical chemical and ECG investigations were performed before the first treatment day and at termination of the treatment and at the end of the recovery period. The blood samples for kinetic analysis were taken in the 2^{nd} and in the 104^{th}, 110^{th} and 111^{th} treatment days. The opthalmoscopic examinations were performed prior to the treatment, at termination of the treatment and at the end of the recovery period. Terminally gross necropsy and histopathological examinations were performed in all animals.

The following results were obtained. No animals died during the study.

The test item Compound I caused vomiting, salivation, thin feces, hypoactivity, tremor, convulsive legs, in-coordination and fear.

The frequency and duration of vomiting, salivation, thin feces, hypoactivity and tremor showed dose dependency in doses of 80 and 160 mg/kg/day.

The convulsive legs, in-coordination and fear as clinical symptoms were observed only in high dose groups.

The mean body weight of the male and female dogs treated at 160 mg/kg dose level showed slight reversible decrease at the end of the treatment period.

The mean food consumption of the female dogs was slightly below the control level in the 160 mg/kg/day dose group. This alteration could be in connection with the test-item treatment.

No ECG and opthalmological alterations were found during the study.

Hematological investigation revealed a slight but statistically and biologically significant decrease in RBC count and in HGB and HTC values in males and females in the high dose group which could be in connection with the reversible effect of the test item.

Clinical chemistry investigation and urinalysis revealed slight changes of a few parameters like cholesterol, total protein, albumin concentrations and specific gravity of urine within physiological range that were probably not in relation to the effect of test item.

No alterations related to the test item effect were found at the macroscopic and microscopic examinations of organs and tissues during the necropsy.

The test item Compound I did not influence the bone marrow function of dogs at dose level 50, 80 and 160 mg/kg during this study.

Compound I shows straight kinetics in dogs within the dose range of 50-160 mg/kg. The Cₘₐₓ and AUCₜₒₜ, values increase straightly with the dose, the clearance t_{1/2} and MRT remain constant. Male and female animals show the same kinetic properties.

Under the present experimental conditions the NOEL is 50 mg/kg/day in both sexes.

### Example 15

### 180-Day Repeated Dose Oral Toxicity Study in Rats

A six-month repeated dose oral toxicity study of Compound I was performed in rats involving a control (physiologic saline, n=32/sex) and dose groups of 200 mg/kg/day, 400 mg/kg/day and 900 mg/kg/day (equal to 124 mg/kg, 248 mg/kg and 558 mg/kg free base, n=20/sex, 20/sex and 32/sex, respectively). The test item was dissolved in distilled water. Treatment was carried out by a stomach tube daily. The application volume was adjusted weekly according to the animal's body weight changes.

Clinical observations were made once daily. Body weight and food consumption were measured and evaluated weekly. Urine collection, blood sampling for hematological, clinical chemistry evaluation and gross necropsy were conducted at the end of the treatment period. Full histopathologic examination was performed on the preserved organs and tissues of the vehicle control and high dose groups. In addition, the liver, kidneys, testes, epididymis and organs showing macroscopic alterations were examined microscopically in the low and medium doses.

Compound I content (pre-dose concentration) was determined in serum on days 1, 49, 111 and 173. The serum samples were analyzed by liquid/liquid extraction, and ion-pair HPLC. Twelve animals per sex of the controls and high dose groups were kept alive for a 28-day post-treatment recovery period. They were processed in the same way as animals at the termination of treatment.

The following results were obtained. No test item related mortality occurred.

No test item influence appeared in the clinical symptoms, body weight and body weight gain data.

A slight increase was observed in daily food intake at 400 mg/kg/day (female) and 900 mg/kg/day (male and female).

No ocular alterations were observed in male and female animals involved in the study.

There were no treatment related effects on the hematological parameters examined.

The cholesterol (male and female group of 900 mg/kg/day) and bilirubin (male at 400 mg/kg/day and 900 mg/kg/day, female at 900 mg/kg/day) levels were elevated at the end of the treatment. The cholesterol concentration remained above the control value at the end of the recovery period in male animals, as well.

The urinalysis revealed significant proteinuria both in male and female animals which was relevant at dose level of 900 mg/kg/day. In male animals it was connected with slightly increased specific gravity and decreased pH.

Test item related gross findings were found in the liver and kidneys. Enlargement of the liver was noted at dose level of 400 mg/kg/day (male) and at 900 mg/kg/day (males and females). Frequency of pale kidneys was five fold and six fold higher than in the control in dose groups of 400 mg/kg/day and 900 mg/kg/day, respectively, both in male and female animals. Atrophy of testes and epididymis occurred only in the high dose group terminally (2/20) and at the end of the recovery period (1/12).

These gross observations correlated with the organ weight data. Significant increase occurred in the liver weight (male and female) at 400 mg/kg/day and 900 mg/kg/day and in the kidney weight (male and female) treated with 900 mg/kg/day. The liver weight increase at 400 mg/kg/day and 900 mg/kg/day (male and female) was not completely reversible. There were no histological lesions associated with the liver weight changes.

In the kidney, microscopic examinations revealed chronic nephropathy in both control and treated animals, however, the incidence was higher in the 400 mg/kg/day and 900 mg/kg/day dose groups (male 14/20 and 14/20; female: 6/20 and 8/20, respectively) in contrast with the control group (male: 4/20, female: 3/20). In the recovery group of 900 mg/kg/day, the incidence was also higher (8/12 male and 4/12 female in contrast with the control 1/12 male and 0/12 female). The 400 mg/kg/day and 900 mg/kg doses of test item could be a predisposing factor for the pathogenesis of chronic nephropathy.

Atrophy of germinal cell layers in the testes and the lack of mature spermatocytes in the epididymis were observed. These alterations were considered individual disorders because of the low incidence. However, a test item influence could not be excluded entirely. No accumulation of Compound I was found after the six month oral application. The serum levels were very low and were only measurable in dose group of 200 mg/kg/day both in male and female animals during the entire study. An enzyme induction might be presumed at 900 mg/kg/day since the serum levels at the end of the treatment were below the values on Day 1.

Under the present experimental conditions the NOEL is 200 mg/kg/day in both sexes.

### Example 16

### Genotoxicity in Bacterial Assays

Seven, 312-5000 μg/plates (equal to 194, 388, 775, 1551 and 3101 μg/plates free base) and two separate experiments were performed using Salmonella typhimurium strains TA98, TA100, TA1537, TA1535 and a strain of Escherichia coli (Escherichia coli WP2 uvrA) in the presence and in the absence of rat liver fraction (3 plates/concentration).

The test item Compound I had no mutagenic activity at concentrations up to 5000 μg test item/plate.

### Example 17

### Genotoxicity in CHO Assays

The test item Compound I was examined at concentrations of 200, 800 and 1400 μg/ml (equal to 124, 496 and 868 μg/ml free base) in the presence and in the absence of metabolic activation (ongoing study). Compound I proved to be non-clastogenic in this metaphase chromosome aberration assay in Chinese Hamster Ovary cells.

### Example 18

### Genotoxicity in Micronucleus Test

On the basis of acute oral toxicity of test item Compound I in CRL: NMRI BR mice the dose level 5000 mg/kg (equal to 3101 mg/kg free base) was examined in the micronucleus test.

The test item did not induce a significant increase in the number of the micronucleated polychromatic erythrocytes (MPCEs) at 5000 mg/kg dose level after single administration (in 24^{th}, 48^{th} hours after the treatment) in male and female mice.

The test item Compound I proved to be non-mutagenic in this in vivo model. In conclusion, test item Compound I tested in vitro both with and without metabolic activation and in vivo proved to be non-mutagenic.

### Example 19

### Single Ascending Dose First-Into-Man Study of Oral Administrations of Compound I in Male Healthy Volunteers

All human studies were conducted according to the study protocols in accordance with GCP.

The primary objective of the study was to assess the pharmacokinetics of Compound I after single ascending oral doses. Compound I was applied in six different doses in two groups of volunteers. Group A had 4 treatment levels (single doses of 50, 200, 400 and 800 mg) and Group B had 3 treatment levels (single doses of 100, 400 and 600 mg). There was at least a 6-day wash-out period after applying each dose.

Descriptive pharmacokinetic statistics by doses are presented in Table 8.

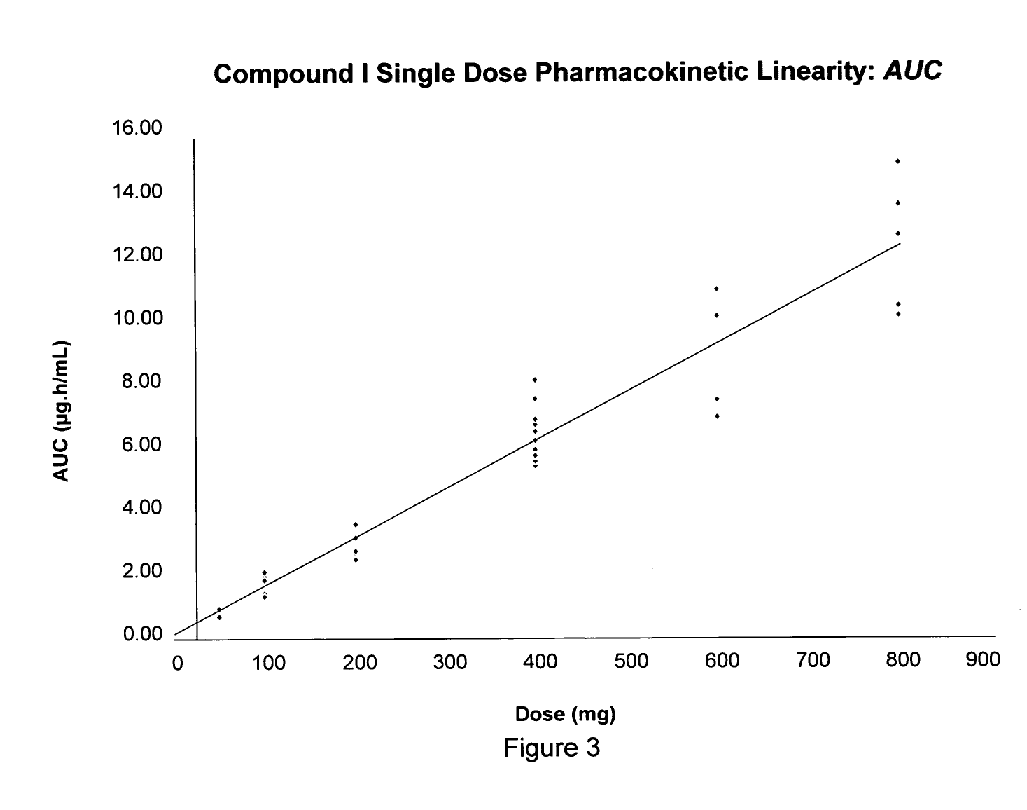

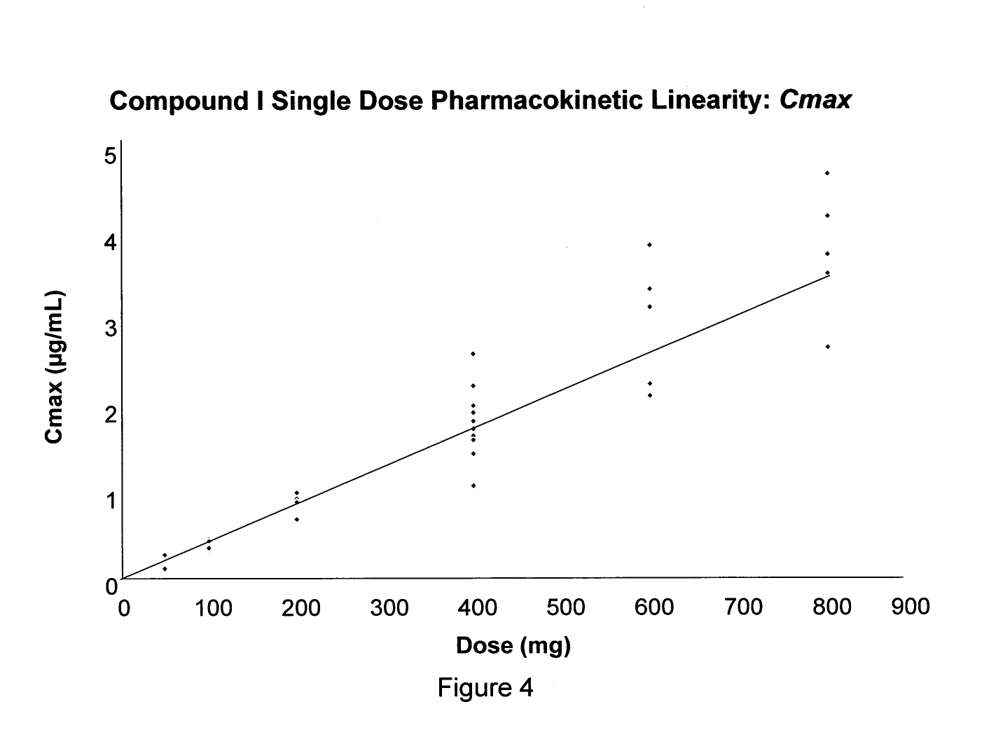

Compound I was absorbed rapidly with Tₘₐₓ values ranged between 0.5 and 1.1 h. Mean t_{1/2} values ranged between 2.5 and 6.2 h. There was a good dose-proportional increase in AUC and Cₘₐₓ values. See, FIGS. 3 and 4.

TABLE 8
Descriptive pharmacokinetic parameters of Compound I.

| | | % | | | | | |
|---|---|---|---|---|---|---|---|
| | Mean | CV | Stdev | N | Min | Max | GeoMean |
| | dose: | | | | | | |
| | 50 mg (group A) | | | | | | |
| Cmax (μg/mL) | 0.17 | 42 | 0.069 | 5 | 0.13 | 0.29 | 0.16 |
| Tmax (h) | 0.9 | 46 | 0.42 | 5 | 0.5 | 1.5 | 0.82 |
| t½ (h) | 3.1 | 15 | 0.47 | 5 | 2.6 | 3.8 | 3 |
| AUC(0-t) (μg · | 0.6 | 19 | 0.12 | 5 | 0.48 | 0.76 | 0.59 |
| h/mL) | | | | | | | |
| AUC (μg · h/mL) | 0.65 | 16 | 0.11 | 5 | 0.56 | 0.81 | 0.64 |
| MRT (h) | 3.5 | 14 | 0.5 | 5 | 2.8 | 4 | 3.4 |
| | dose: | | | | | | |
| | 200 mg (group A) | | | | | | |
| Cmax (μg/mL) | 0.89 | 13 | 0.12 | 5 | 0.7 | 1 | 0.89 |
| Tmax (h) | 0.9 | 46 | 0.42 | 5 | 0.5 | 1.5 | 0.82 |
| t½ (h) | 3.1 | 18 | 0.57 | 5 | 2.6 | 4.1 | 3.1 |
| AUC(0-t) (μg · | 2.7 | 18 | 0.49 | 5 | 2.2 | 3.4 | 2.7 |
| h/mL) | | | | | | | |
| AUC (μg · h/mL) | 2.8 | 16 | 0.45 | 5 | 2.4 | 3.5 | 2.8 |
| MRT (h) | 3.8 | 30 | 1.1 | 5 | 3.1 | 5.9 | 3.7 |
| | dose: | | | | | | |
| | 400 mg (group A and B) | | | | | | |
| Cmax (μg/mL) | 1.8 | 23 | 0.42 | 10 | 1.1 | 2.6 | 1.8 |
| Tmax (h) | 0.9 | 44 | 0.39 | 10 | 0.5 | 1.5 | 0.82 |
| t½ (h) | 4.1 | 12 | 0.51 | 10 | 3.4 | 4.7 | 4 |
| AUC(0-t) (μg · | 6.3 | 14 | 0.85 | 10 | 5.3 | 7.8 | 6.2 |
| h/mL) | | | | | | | |
| AUC (μg · h/mL) | 6.4 | 14 | 0.88 | 10 | 5.3 | 8 | 6.3 |
| MRT (h) | 4.5 | 14 | 0.62 | 10 | 3.7 | 5.7 | 4.5 |
| | dose: | | | | | | |
| | 800 mg (group A) | | | | | | |
| Cmax (μg/mL) | 3.8 | 20 | 0.75 | 5 | 2.7 | 4.7 | 3.7 |
| Tmax (h) | 0.8 | 34 | 0.27 | 5 | 0.5 | 1 | 0.76 |
| t½ (h) | 3.5 | 19 | 0.66 | 5 | 3 | 4.6 | 3.4 |
| AUC(0-t) (μg · | 12 | 17 | 2 | 5 | 10 | 15 | 12 |
| h/mL) | | | | | | | |
| AUC (μg · h/mL) | 12 | 17 | 2.1 | 5 | 10 | 15 | 12 |
| MRT (h) | 4.1 | 13 | 0.54 | 5 | 3.4 | 4.7 | 4.1 |
| | dose: | | | | | | |
| | 100 mg (group B) | | | | | | |
| Cmax (μg/mL) | 0.4 | 15 | 0.058 | 5 | 0.34 | 0.47 | 0.39 |
| Tmax (h) | 1.1 | 50 | 0.55 | 5 | 0.5 | 2 | 1 |
| t½ (h) | 3.7 | 39 | 1.5 | 5 | 2.5 | 6.2 | 3.5 |
| AUC(0-t) (μg · | 1.4 | 17 | 0.24 | 5 | 1.2 | 1.7 | 1.4 |
| h/mL) | | | | | | | |
| AUC (μg · h/mL) | 1.6 | 22 | 0.35 | 5 | 1.2 | 2 | 1.6 |
| MRT (h) | 3.8 | 12 | 0.46 | 5 | 3.1 | 4.2 | 3.7 |
| | dose: | | | | | | |
| | 600 mg (group B) | | | | | | |
| Cmax (μg/mL) | 3 | 25 | 0.74 | 5 | 2.1 | 3.9 | 2.9 |
| Tmax (h) | 0.5 | 0 | 0 | 5 | 0.5 | 0.5 | 0.5 |
| t½ (h) | 4.3 | 9.7 | 0.41 | 5 | 3.7 | 4.7 | 4.2 |
| AUC(0-t) (μg · | 9.1 | 21 | 1.9 | 5 | 6.8 | 11 | 8.9 |
| h/mL) | | | | | | | |
| AUC (μg · h/mL) | 9.2 | 21 | 2 | 5 | 6.9 | 11 | 9 |
| MRT (h) | 4 | 11 | 0.43 | 5 | 3.4 | 4.4 | 3.9 |

### Example 20

### Double-Blind Multiple Dose Study of Oral Administrations of Compound I in Male Healthy Volunteers

The study was designed to compare the pharmacokinetic profile of Compound I from the following dose regimens:
A: Administration of 1 capsule containing 50 mg of Compound I or of 1 capsule of placebo, three times a day, total dose 150 mg.
B: Administration of 1 capsule containing 100 mg of Compound I or of 1 capsule of placebo, three times a day, total dose 300 mg.

The treatments were applied in parallel groups. The study was performed in 18 healthy subjects divided into 2 groups of 9 subjects (groups A and B). Subjects of group A received 50 mg Compound I as a single dose on the morning of Day 1; then 50 mg Compound I tid on Days 2 to 9; and a single 50 mg dose on the morning of day 10. Subjects of group B were treated with 100 mg Compound I in a similar regimen as per group A. Three subjects in each group received placebo. These subjects were not considered for the pharmacokinetic evaluation. The study was conducted according to the study protocol and in accordance with GCP.

The design of the study was adequate: (1) to determine the relative ADME characteristics, including dose proportionality, from two different doses and, (2) to compare the multiple dose pharmacokinetics with the single dose pharmacokinetics in order to assess the accumulation rate and to evaluate the linearity of pharmacokinetics.

If dose proportionality applies, the expectation for the dose-adjusted AUC and Cₘₐₓ ratios (2×50 mg dosing level/100 mg dosing level) will be 1.00 at both Day 1 and Day 10. According to Table 9.6, the dose-adjusted AUC_{0-8 h}'s are 0.95 and 1.02, on Day 1 and Day 10, respectively. Similarly, the dose-adjusted Cₘₐₓ's are 0.94 and 0.88 on Day 1 and Day 10, respectively. Thus AUC_{0-8 h} and Cₘₐₓ are approximately dose-proportional at the doses and time intervals tested.

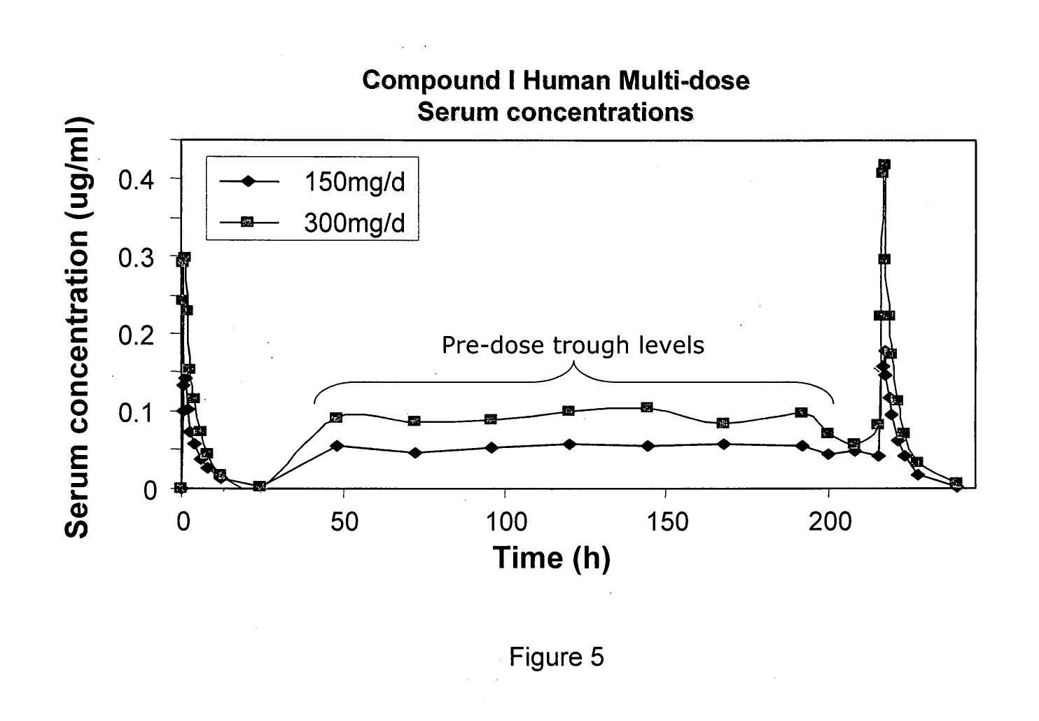

The results are presented in FIG. 5 and Tables 8a and 9. Multiple doses separated by a dosage interval of 8 h (tid) resulted in a multi-dose increasing ratio (R_{inc}=AUC_{0.8 Day 10}/AUC_{0-8 Day 1}) of 1.4 to 1.5 at steady-state, independently from the dose (Table 9). Following daily three time administrations the pre-dose concentrations became different from zero. The morning trough concentrations were 50-60 ng/ml after A and 90-100 ng/ml after B and after 48 h they showed no trend. It may thus be concluded that the subjects were in a steady state.

TABLE 8a
Compound I Human Multi-Dose Pharmacokinetic Parameters

| | AUC0-8 h | | | | | |
|---|---|---|---|---|---|---|
| | (μg × h/ml) | | Cmax (μg/ml) | | t½ (h) | |
| Dose time | 150 mg | 300 mg | 150 mg | 300 mg | 150 mg | 300 mg |
| First dose | 0.52 | 1.10 | 0.17 | 0.36 | 4.2 | 3.4 |
| Last dose | 0.79 | 1.54 | 0.21 | 0.47 | 4.1 | 4.2 |

After multiple doses the tₘₐₓ and elimination half-lives remained unchanged and only the peak concentrations were shifted due to the non-zero pre-dose values. Thus, under steady state conditions, the total exposures separated by a dosage interval after multiple doses (AUC_{0-8 h Day 10}) were about 40-50% higher compared to those after single administration (AUC_{0-8 h Day 1}) (see r_{acc}, Table 9).

TABLE 9
Compound I pharmacokinetic variables.
Compound I PK-Variables - Treatment A

| | Day-1 Dose: 50 mg Compound I | | | | | | | Day-10 dose: 50 mg Compound I tid | | | | | |
|---|---|---|---|---|---|---|---|---|---|---|---|---|---|
| | AUC_{0-8 h} | AUC_{0-tz} | AUC_{0-inf} | | Cₘₐₓ | | | AUC_{0-8 h} | Cₘₐₓ | | | | |
| Subj. | μg/ml * h | μg/ml * h | μg/ml * h | rAUC % | μg/ml | tₘₐₓ h | t_{1/2} h | μg/ml * h | μg/ml | tₘₐₓ h | t_{1/2} h | R_{acc} | LinPK |
| N | 6 | 6 | 6 | 6 | 6 | 6 | 6 | 6 | 6 | 6 | 6 | 6 | 6 |
| MEAN | 0.520 | 0.632 | 0.691 | 8.4 | 0.170 | 0.92 | 4.22 | 0.786 | 0.208 | 217.00 | 4.14 | 1.525 | 1.166 |
| STD | 0.068 | 0.127 | 0.132 | 7.0 | 0.028 | 0.49 | 1.02 | 0.091 | 0.039 | 0.55 | 1.28 | 0.226 | 0.230 |
| GeoM | 0.517 | 0.621 | 0.680 | 5.9 | 0.168 | | 4.12 | 0.781 | 0.205 | | 3.99 | 1.512 | 1.148 |
| GeoCV | 13.6 | 20.5 | 19.5 | 130.6 | 17.7 | | 22.9 | 12.6 | 18.6 | | 29.7 | 14.1 | 19.3 |

Compound I PK-Variables - Treatment B

| | Day-1 dose: 100 mg Compound I | | | | | | | Day-10 dose: 100 mg Compound I tid | | | | | |
|---|---|---|---|---|---|---|---|---|---|---|---|---|---|
| | AUC₀₋₈ | AUC_{0-tz} | AUC_{0-inf} | | Cₘₐₓ | | | AUCₜ | Cₘₐₓ | | | | |
| Subj. | μg/ml * h | μg/ml * h | μg/ml * h | rAUC % | μg/ml | tₘₐₓ h | t_{1/2} h | μg/ml * h | μg/ml | tₘₐₓ h | t_{1/2} h | R_{acc} | LinPK |
| N | 6 | 6 | 6 | 6 | 6 | 6 | 6 | 6 | 6 | 6 | 6 | 6 | 6 |
| MEAN | 1.101 | 1.307 | 1.344 | 2.8 | 0.360 | 0.92 | 3.43 | 1.540 | 0.473 | 217.00 | 4.19 | 1.426 | 1.187 |
| STD | 0.241 | 0.361 | 0.367 | 1.5 | 0.046 | 0.38 | 0.94 | 0.214 | 0.087 | 0.45 | 1.00 | 0.194 | 0.209 |

Compound I PK-Variables - Treatment A

| | Day-1 Dose: 50 mg Compound I | | | | | | | Day-10 dose: 50 mg Compound I tid | | | | | |
|---|---|---|---|---|---|---|---|---|---|---|---|---|---|
| | AUC_{0-8 h} | AUC_{0-tz} | AUC_{0-inf} | | Cₘₐₓ | | | AUC_{0-8 h} | Cₘₐₓ | | | | |
| Subj. | μg/ml * h | μg/ml * h | μg/ml * h | rAUC % | μg/ml | tₘₐₓ h | t_{1/2} h | μg/ml * h | μg/ml | tₘₐₓ h | t_{1/2} h | R_{acc} | LinPK |
| GeoM | 1.079 | 1.266 | 1.303 | 2.5 | 0.358 | | 3.32 | 1.527 | 0.466 | | 4.10 | 1.415 | 1.172 |
| GeoCV | 22.3 | 27.9 | 27.8 | 66.3 | 12.6 | | 27.9 | 14.6 | 19.5 | | 21.9 | 13.3 | 17.3 |

R_{acc} accumulation ratio = AUC_{0-8 Day 10}/AUC_{0-8 Day 1;}
LinPK = AUC_{0-8 Day 10}/AUC_{0-inf}

### Example 21

### Single Ascending Dose First-Into-Man Study of Oral Administrations of Compound I in Male Healthy Volunteers

The study was designed as a randomized, double-blind and single-center study, without therapeutic benefit for the subjects. The objective of the study was to assess the safety of single ascending oral doses of Compound I in healthy young male subjects. The study was conducted in 12 volunteers, in accordance with the Helsinski Declaration, European Good Clinical Practices and Huriet law. The volunteers were divided into two groups (n=6 in each group). Compound I was applied in six different doses in these two groups of volunteers. Group A had 4 treatment periods (doses 50, 200, 400 and 800 mg) and Group B had 3 treatment periods (doses 100, 400 and 600 mg). Randomization, at each dose level investigated, was in the ratio of 1 placebo to 5 active treatments (5:1). There was at least a 6-day wash-out period after applying each dose.

The dose was administered orally, under medical supervision, after a 10 h abstinence from food. No concomitant medication was allowed within the period of time from the screening examination to 24 h after the last administration.

There were no serious adverse events or deaths reported during the study.

Two subjects reported mild sleepiness (with an approx. 2-2.5 h duration) after administration of the 400 mg dose. The duration of these events were 2.25 and 5.50 h and they were stated by the investigators as “possibly related” to the study treatment. However, after unblinding it has been found that one of these patients was treated with placebo at that dose level. Following these events both patients continued the study and took the capsules of higher dose levels without any adverse event. No changes in any safety parameters (such as laboratory parameters, vital signs, or ECGs) were reported during the study. Therefore, the safety of the compound was considered as very good.

### Example 22

### A Double-Blind Multiple Dose Study of Oral Administrations of Compound I in Male Healthy Volunteers

The study was designed as a randomized, double-blind and single-center study, without therapeutic benefit for the subjects. The objective of the study was to assess the safety of multiple oral doses of Compound I in healthy young male subjects.

The study was performed in 18 healthy subjects divided into 2 groups of 9 subjects (groups A and B). Subjects of group A received 50 mg Compound I as a single dose on the morning of day 1; then 50 mg Compound I tid on days 2 to 9; and a single 50 mg dose on the morning of day 10. Subjects of group B were treated with 100 mg Compound I in a similar regimen as per group A. Randomization, at each dose level investigated, was in the ratio of 3 placebo to 6 active treatments.

There were no serious adverse events or deaths were reported during the study. Compound I was generally well tolerated by the study subjects. Fourteen subjects reported 31 adverse events. Seven events were reported by the subjects receiving placebo, 11 adverse events were reported by subjects receiving the 150 mg Compound I treatment and 20 adverse events were reported on the 300 mg Compound I treatment. The intensity of these events was rated as mild to moderate. No clinically significant abnormal values were reported in the vital signs or ECG assessments. There were no statistically significant changes in laboratory parameters. Three abnormal hematology values in 3 subjects were stated as “clinically significant” (leukocytes at screening in subject No. 11; eosinophils at day 11 in subject No. 2 [who showed elevated eosinophil values from screening and was treated with 3×50 mg Compound I]; and eosinophils at day 5 in subject No. 6 [the value decreased to day 10 and this subject was treated with placebo]). However, although changes in serum creatinine levels were mentioned neither by the investigators nor by the statistical evaluation, it is fair to point out that modest increases were observed in a number of volunteers. The increases were within the clinically accepted normal range and disappeared after completion of the dosing regimen. Similar increases followed by a return to initial levels were observed during clinical trials of bimoclomol, the parent compound of Compound I, and appeared to have no safety implications.

Thus, no clinically significant adverse events were observed, nor were any significant changes seen in laboratory parameters, vital signs, or ECGs.

### Example 23

### Safety and Pharmacokinetics Study of Compound I in ALS

A clinical trial was conducted to assess the safety and tolerability of Compound I, at three dosages (75, 150, and 300 mg/day) as compared with placebo over 12 weeks of treatment in 80 patients with ALS. It was also conducted to determine the pharmacokinetic characterization of Compound I in serum, as well as cerebrospinal fluid (CSF) penetration, in a subset of the 80 patients participating in the study. This information was correlated with safety measures.

The study rational and significance: Compound I is a small molecule that upregulates heat shock proteins in cells under stress. When given both pre-symptomatically and at disease onset in a mutant superoxide dismutase transgenic mouse model of ALS, Compound I extends survival by five weeks. Compound I delays the death of motor neurons in treated mice and delays the associated loss of motor unit potentials. The effect of Compound I is greater than that found with most other compounds, including riluzole and minocycline, when tested in this in vivo model of ALS.

ALS is a severe and ultimately fatal disease, for which there is no known effective treatment. Any compound proven to slow the course of the illness will be of immediate importance clinically; moreover, a positive outcome will enhance our understanding of the underlying biology of ALS.

Methodology: This study was a multicenter, double-blind, placebo-controlled study of outpatients with ALS. Eighty subjects at 8-10 centers were enrolled. Subjects received placebo, 25 mg tid, 50 mg tid or 100 mg tid Compound I daily. All 80 subjects received treatment to determine safety and tolerability after 12 weeks of daily treatment. Follow-up visits occurred at 2, 4, 6, 8, 10, 12, and 16 weeks. A subset of participants were admitted to the General Clinical Research Center (GCRC), clinical research center or other department for serum pharmacokinetic studies at Baseline and Week 4, with additional trough serum sampling at Weeks 2, 8, and 12. Cerebrospinal fluid (CSF) penetration was only be evaluated at Week 4.

Tolerability: Compound I was well tolerated at all three doses tested. Tolerability was determined by the number of patients who did not finish the 12-week study in each dose group. In the 12-week dosing period, the number of patients who did not complete dosing at each of the doses was one (4.5%), two (10%), zero (0%), and three (13.7%) in the placebo, 75 mg/day, 150 mg/day, and 300 mg/day groups, respectively. The time to early dose discontinuation was not significantly affected by dose.

Safety: There were no statistically significant (p<0.05) drug related adverse events or serious adverse events. ASTHENIA (weakness) events decreased marginally with increasing treatment dose, six (27%) patients with ASTHENIA on placebo, five (25%) on 75 mg/day, one (5%) on 150 mg/day, two (9%) on 300 mg/day, p=0.053. There was a patient on 75 mg/day having the ASTHENIA event twice, when this was counted this effect became statistically significant, p=0.047. There were statistically significant, but clinically irrelevant treatment-related changes in the laboratory results for serum creatinine and for creatinine clearance, two indicators of possible kidney dysfunction. These changes were small, were within the normal range of values, and did not seem to be dose- or time-dependent.

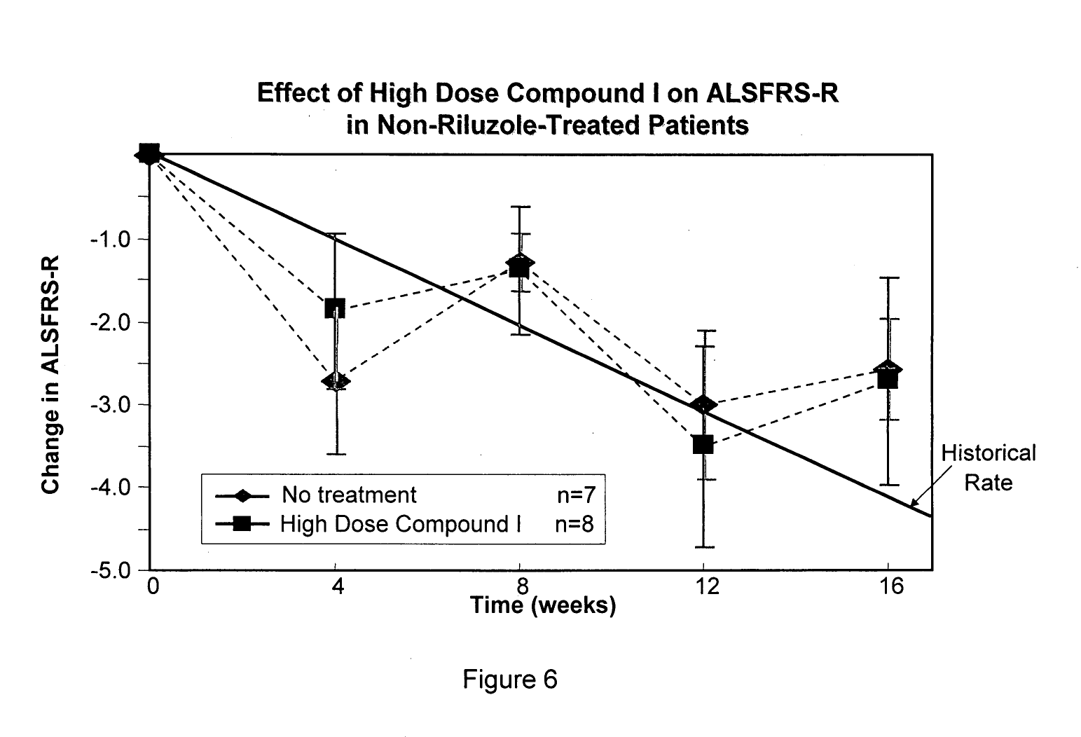

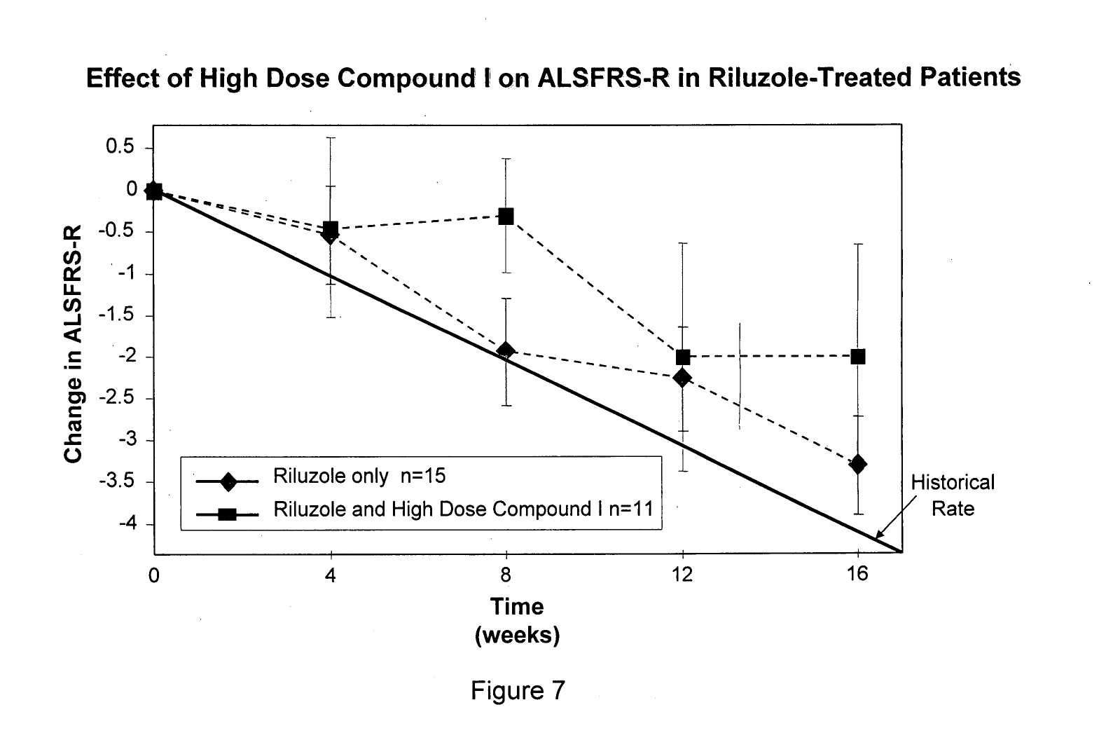

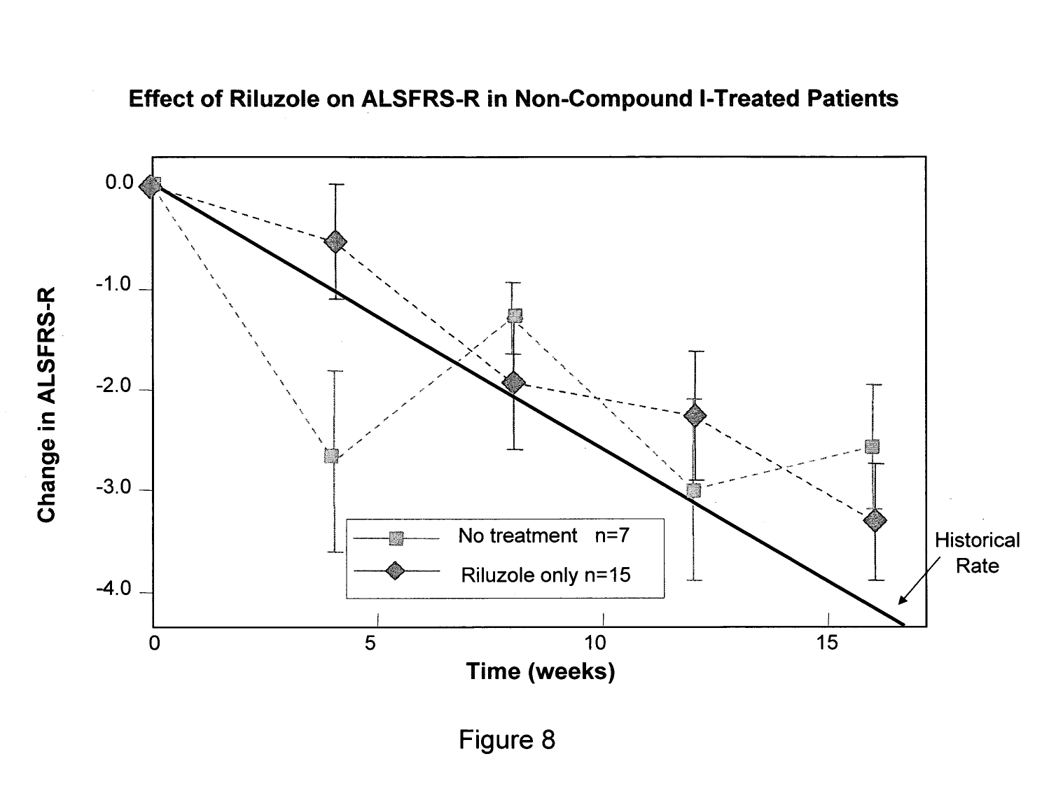

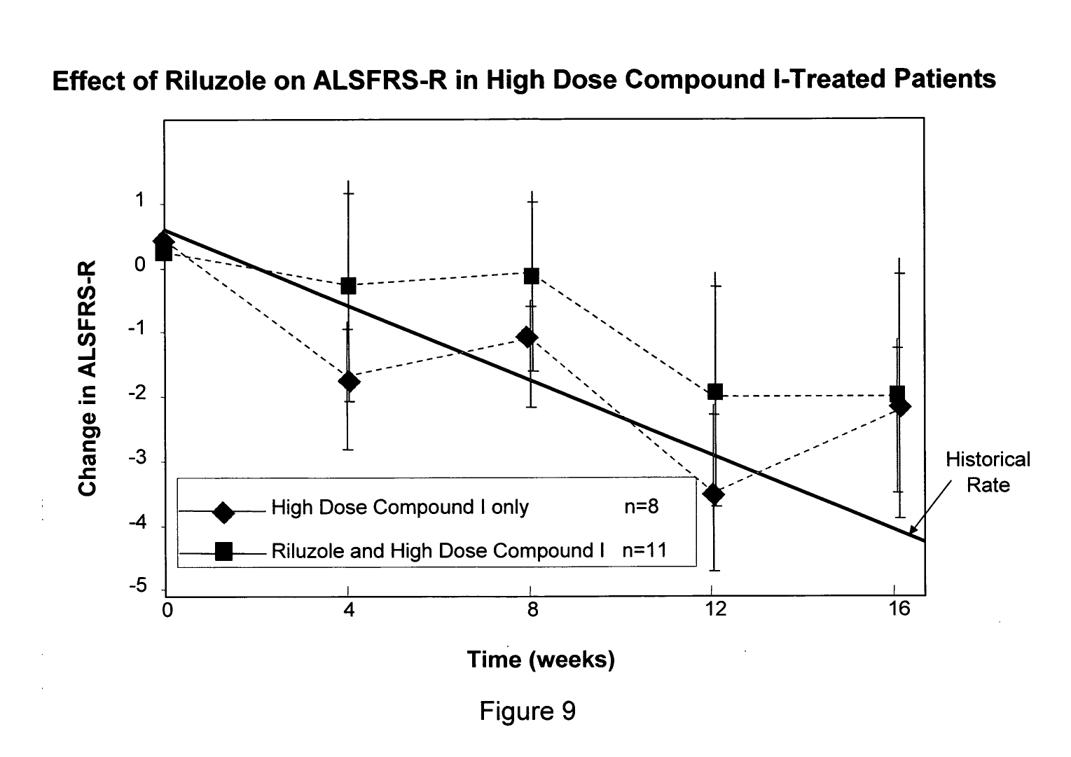

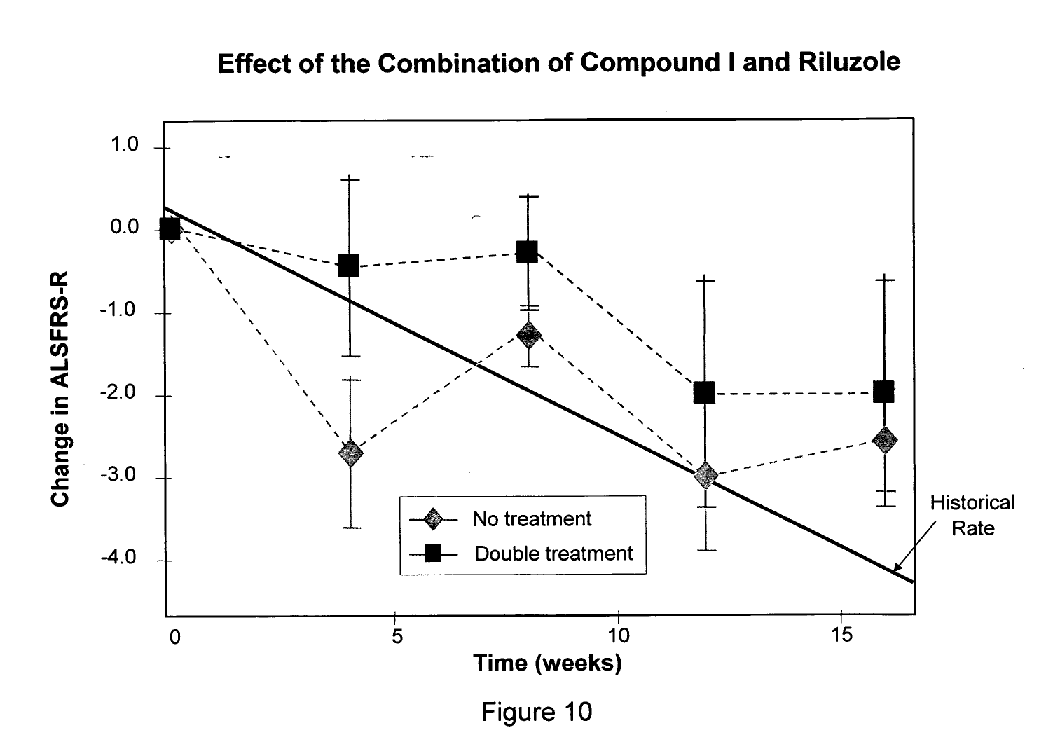

Indicators of disease progression: There were no statistically significant treatment or dose effects in ALSFRS-R (the quantitative “survey”), vital capacity (breath capacity), weight, or body mass index. As depicted in FIG. 6, there was no effect of high dose Compound I on the average ALSFRS-R score in patients who are not treated with riluzole. This effect was also seen in patients receiving riluzole only, as depicted in FIG. 8. Surprisingly, high dose Compound I may improve ALSFRS-R in patients who were also treated with riluzole. See, FIGS. 7 and 9. Thus, the combination of Compound I and riluzole may slow progression of ALS. See, FIG. 10. However, neither of these observations reached statistical significance. No similar apparent riluzole-dependent improvements in vital capacity were observed.

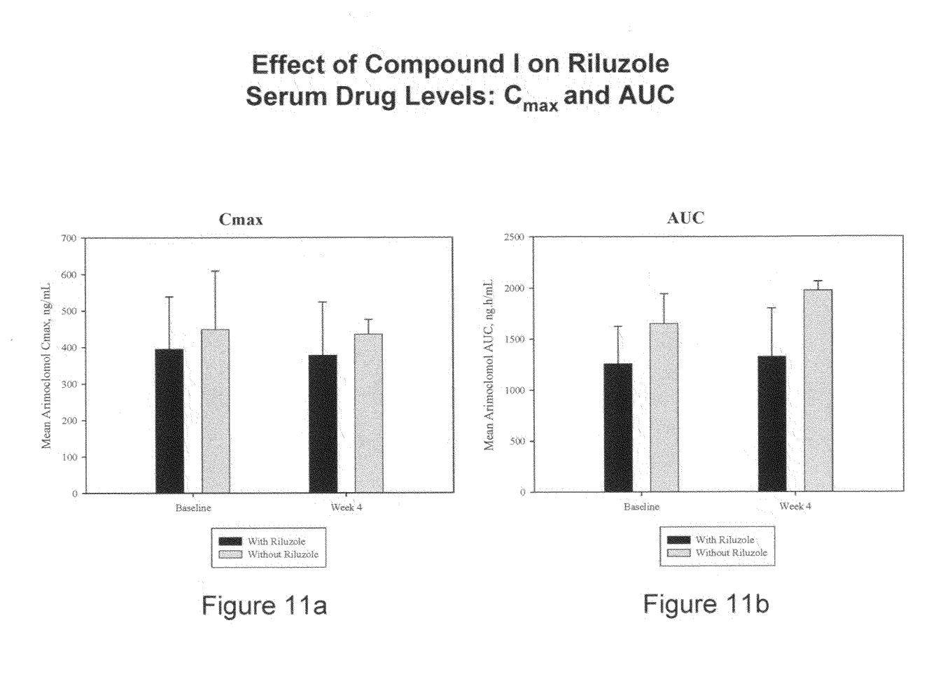

Pharmacokinetics: Although analysis of the samples is not yet complete, preliminary analysis suggests that Compound I crossed the blood:brain barrier in an apparent dose-dependent fashion. The concentrations of drug present in cerebral spinal fluid (the fluid in the spinal cord where the degenerating motor neurons are) were comparable to those in blood. As seen in FIGS. 11a-b, Compound I does not seem to interfere with the blood concentrations of riluzole as determined by either Cₘₐₓ(See FIG. 11a) or AUC (See FIG. 11b).

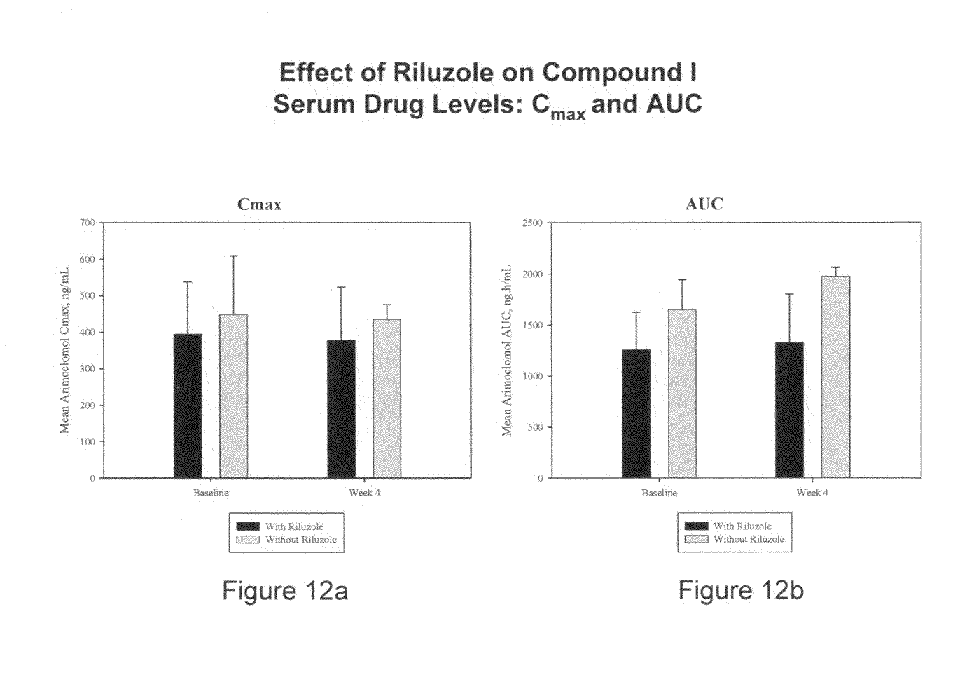

FIGS. 12a-b depict that the effect of riluzole on Compound I serum levels for a 300 mg q.d. group as determined by either Cₘₐₓ(See FIG. 12a) or AUC (See FIG. 12b). These figures indicate that there was virtually no effect of riluzole on the pharmacokinetics of Compound I.

### Example 24

### Functional Recovery Following MCA Occlusion in Rats

In this study, the efficacy of Compound I in enhancing neurological recovery in a model of permanent middle cerebral artery occlusion (MCAO) in rats was tested. Data from this study were reported in U.S. Provisional Application No. 60/920,396, which is incorporated herein by reference. The permanent MCAO is well accepted and considered to be a standard animal model for studying clinical aspects of stroke. (See, e.g., Stroke 1999; 30:2752-2758.)

Animal: 40 male Sprague Dawley Rats, 300-400 g (50 to arrive 7-10 days before surgery at 225-275 g):
10*Drug A: 200 mg/kg/d×3d, then 50 mg/kg/d thereafter, once p.o., qd,

starting at 1 h after MCAO
10*Drug B: 200 mg/kg/d×3d, then 50 mg/kg/d thereafter, once p.o., qd,

starting at 1 h after MCAO

10*Intracisternal bFGF 1 μg; at 1 h and 1 day after MCAO (positive control)
10*Vehicle, once p.o. qd, starting at 1 h after MCAO

Anesthesia: 1-3% halothane or isoflourane in N2O:O2 (2:1)

Temperature: 37.5±0.5° C.

Handling, surgery, injections and sacrifice timetable:

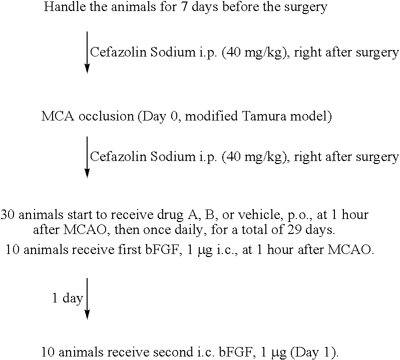

Sacrifice: On day 28 after MCAO, rats are anesthetized deeply with Chloral Hydrate and perfused transcardially with normal saline (with heparin 2 unit/ml) followed by 10% formalin for infarct volume measurement (H&E staining).

Behavioral test timetable:

Limb placing tests:
Evaluation: day-1 (pre-operation), day 1, day 7, then days 14, 21 and 28).

Forelimb placing test (0-12):
whisker tactile placing (0-2);
visual placing (forward, sideways) (0-4);
tactile placing (dorsal, lateral) (0-4);
proprioceptive placing (0-2).

Hindlimb placing test (0-6):
tactile placing (dorsal, lateral) (0-4);
proprioceptive placing (0-2).

Body swing test:

Evaluation: day-1 (pre-operation), day 1, day 7, then days 14, 21 and 28).

Infarct measurement: Sections of (compared to Bregma, respectively) 4.7, 2.7, 0.7, −1.3, −3.3, −5.3 and −7.3 is measured using an image analyzer to get an (indirect) infarct volume.

### Example 25

### Open Label Extension of Safety and Pharmacokinetics Study of Compound I in ALS

An open label extension study was conducted in patients from Example 23 who completed 12 weeks of treatment and a Week 16 Follow-up Visit. Subjects received 100 mg tid Compound I daily for approximately six months. Visits occurred at Screening, Baseline, and once a month for six months (total of 8 visits). A telephone call at Month 7 ended subject participation in the study. Because this study did not have a placebo control, results were compared with data from the placebo group from a Celebrex® trial as a historical control, where possible and appropriate.

Baseline Characteristics: In order to determine the reliability of comparing this study with the prior Celebrex® study, the baseline characteristics of the two studies were compared. The table below indicates that the characteristics of the two populations were similar. The largest differences were in the mean time from symptom onset to diagnosis (24.1% difference), % male sex (17.5%), and mean ALSFRS-R score (11.5%).

Baseline Characteristics for Open-Label Compound I and
Celebrex ® Historical Trial (placebo subjects)

| | Open-Label | Celebrex ® |
|---|---|---|
| | Compound I | Placebo |
| Variable | (n = 69) | (n = 99) |
| Mean age*, yr (SD) | 53.3 (11.6) | 55.0 (12.4) |
| Male sex, % | 57 | 67 |
| Mean time from symptom onset to | 0.87 (0.65) | 1.08 (0.92) |
| diagnosis, yr (SD) | | |
| Taking riluzole*, % | 72 | 71 |
| Mean VC % predicted* (SD) | 81.54 (23.07) | 85.36 (15.13) |
| Mean ALSFRS-R score* (SD) | 38.77 (8.01) | 43.24 (5.17) |

*Open-Label Compound I: age at OL phase screening visit, riluzole treatment from medical history form, VC % predicted and ALSFRS-R from OL phase baseline visit.

Tolerability: Tolerability of treatment with Compound I was estimated by comparing the dropout rate with that of the placebo of the Celebrex® trial. The following table indicates that the percentage dropout rate per month was less for Open-Label Compound I than it was for the placebo for the Celebrex® trial. This suggests that Compound I was extremely well tolerated.

Tolerability by Comparison of
Percent Early Study Medication Discontinuation per Month

| | | | | % Early | # Early Study |
|---|---|---|---|---|---|
| | | Completed | Early Study | Study | Medication |
| | Assigned to | Trial on | Medication | Medication | Discontinuations |
| Study | Treatment | Treatment | Discontinuation | Discontinuation | per Month* |
| Open-Label | 69 | 61 | 8 | 11.6 | 1.33 |
| Compound I | | | | | |
| Celebrex ® | 99 | 65 | 33 | 33.3 | 2.75 |
| Placebo | | | | | |

*over 6-months for Compound 1, 12-months for Celebrex ®

The reasons for early discontinuation of study medication are reported in the following table. In both trials, the most common reason for early study medication discontinuation was withdrawal of consent. It is interesting to note that there were 9 deaths in the placebo group from the Celebrex® trial (with 99 patients treated for 12 months) and only one death in Open-Label study of Compound I (69 patients treated for 6 months).

Reasons for Treatment Discontinuation

| | Adverse | | Withdrew | Other | Reason not |
|---|---|---|---|---|---|
| Study | Event | Death | Consent | Reasons | listed |
| Open-Label | 1 | 1 | 6 | 0 | 0 |
| Compound I | | | | | |
| Celebrex ® Placebo | 4 | 9 | 16 | 7 | 5 |

Safety: Renal safety was assessed by comparing the screening and 6-month laboratory results summarized in the table below. Of note, there was no significant change in serum creatinine levels during this study. This is surprising considering the small but statistically significant increases observed in serum creatinine in all dose groups in the double-blind study that then returned to pre-dose levels.

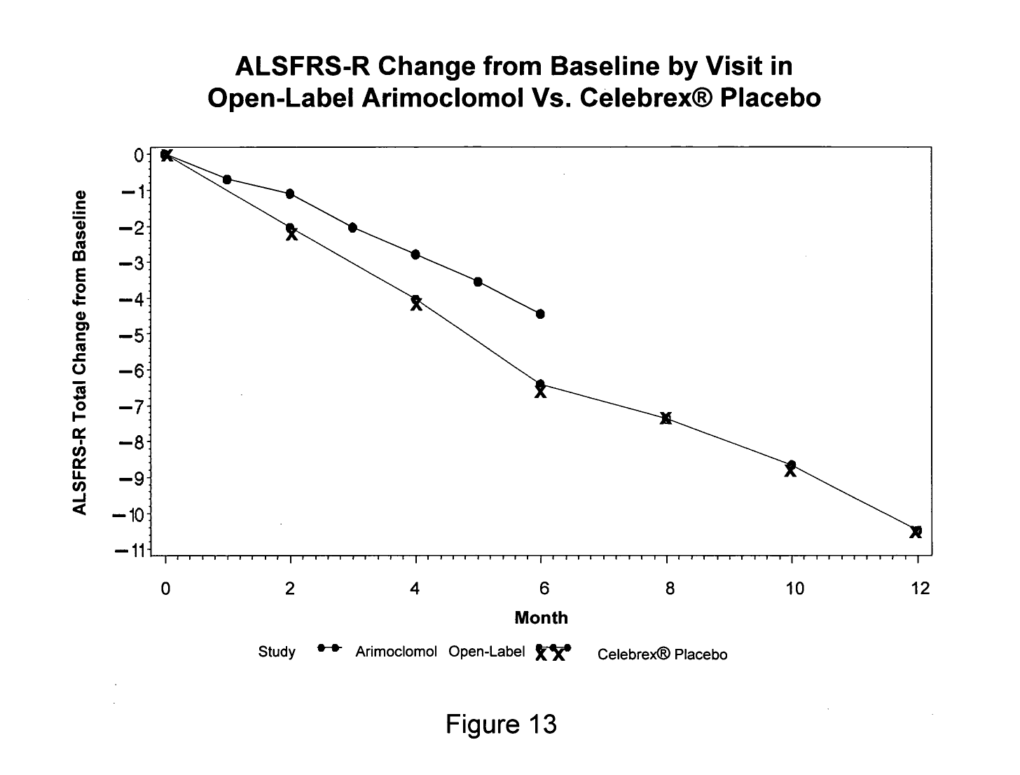

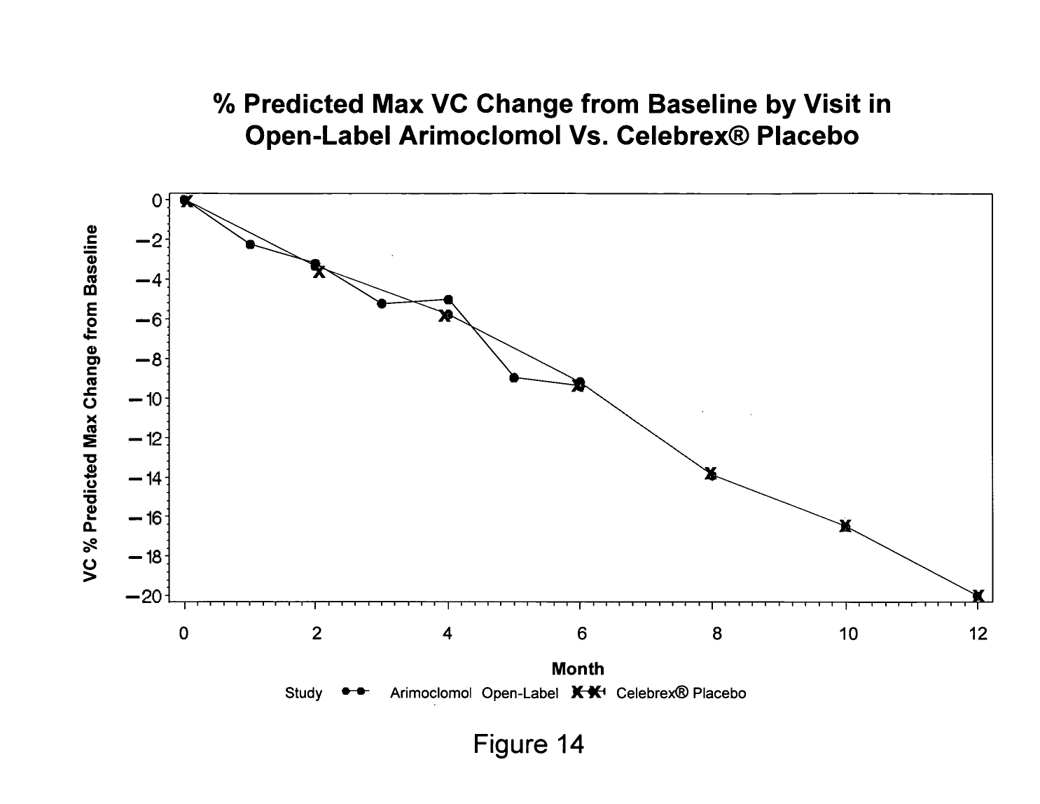

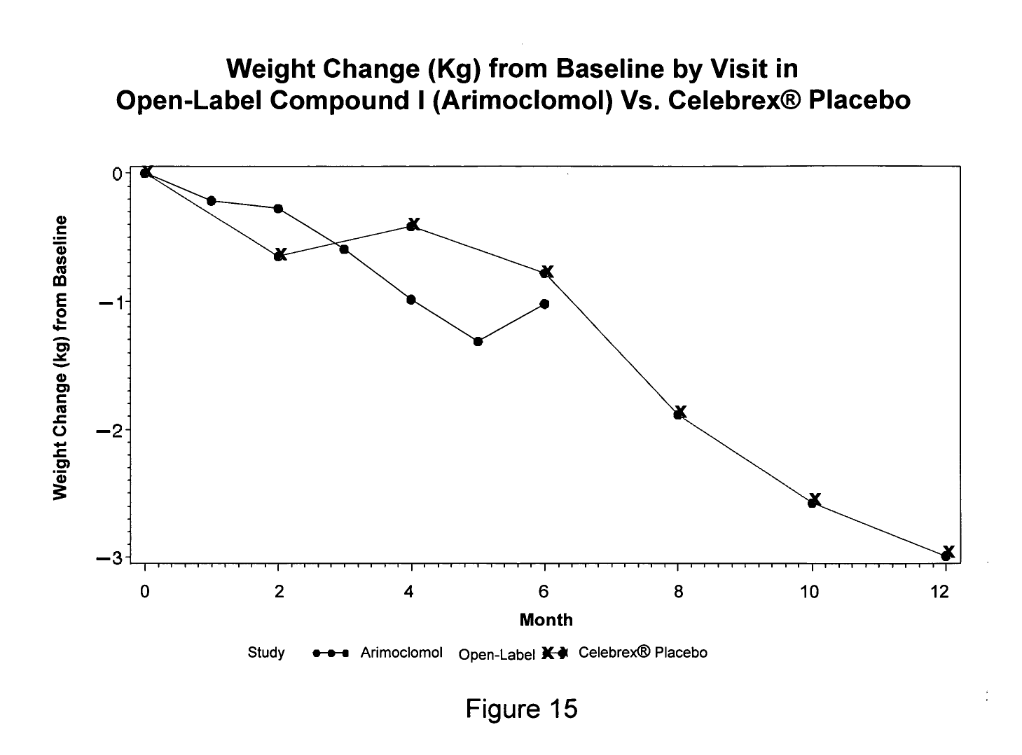

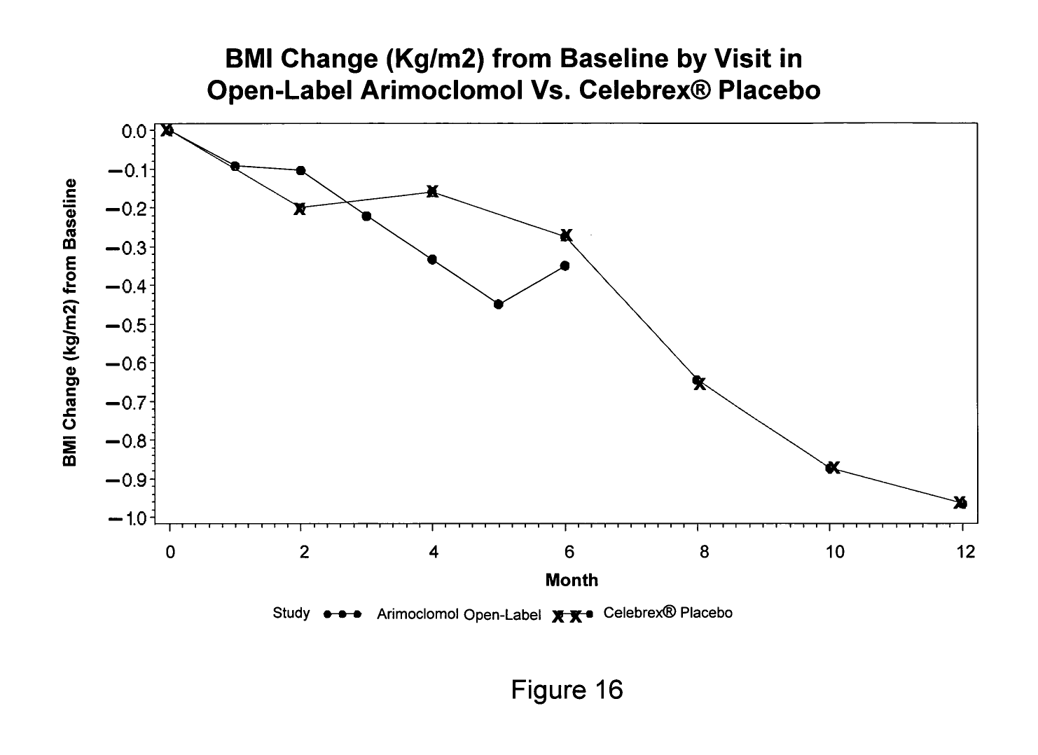

Disease Progression: There are four main indicators of disease progression that can be measured accurately in these time frames; ALSFRS-R, % VC, weight, and body mass index. The analysis assumed that any subject who dropped out (or died) had the worst possible outcome, based on an analysis by Finkelstein and Schoenfeld (Stat. Med. 1999 18:1341-54). This non-parametric analysis orders outcomes so that subjects who dropped out (or died) were considered to have a worse outcome than subjects who lived. For comparison these characteristics for the placebo group in the Celebrex® trial are included. (See FIGS. 13-16). Although there is no way to accurately determine the statistical significance of these comparisons, it is interesting to note that the rate of progression based on slope analysis is lower for every parameter in the Open-Label Compound I study compared with the placebo cohort from the Celebrex® study. Patients in the Compound I trial declined 21.4% slower by ALSFRS-R, 7.6% slower by % VC, 22.8% by weight, and 20.4% by BMI.

Slopes of Outcome Measures
6-Month Treatment for OL Compound I; 12-Month Placebo
for Celebrex ®

| | Open-Label | |
|---|---|---|
| | Compound I | Celebrex ® |
| Outcome | Slope (SE) in | Placebo Slope |
| Measure | (U/mo) | (SE) in (U/mo) |
| ALSFRS-R | −0.8476 (0.1093) | −1.0783 (0.9071) |
| VC % predicted | −2.0285 (0.2807) | −2.1947 (0.2202) |
| Weight (kg) | −0.25923 (0.1023) | −0.3356 (0.07425) |
| BMI (kg/m²) | −0.08987 (0.0347) | −0.1130 (0.0245) |

Overall, 6 month dosing with Compound I was safe and well tolerated. In addition, there are indications that Compound I may be of therapeutic benefit to patients.

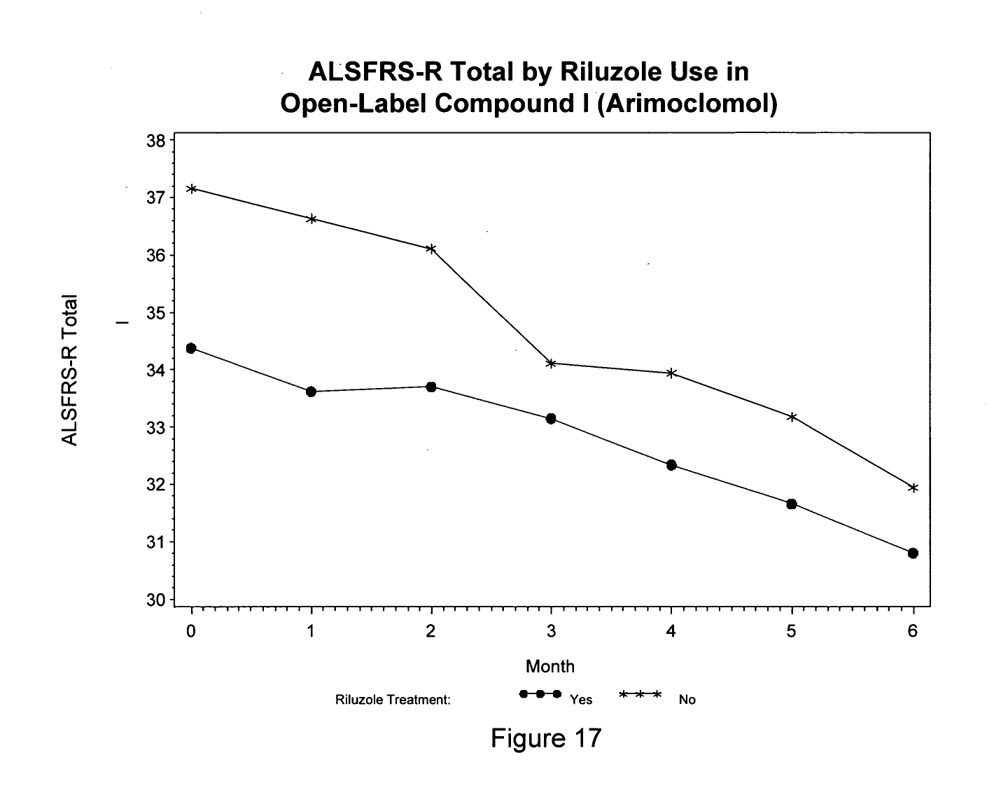

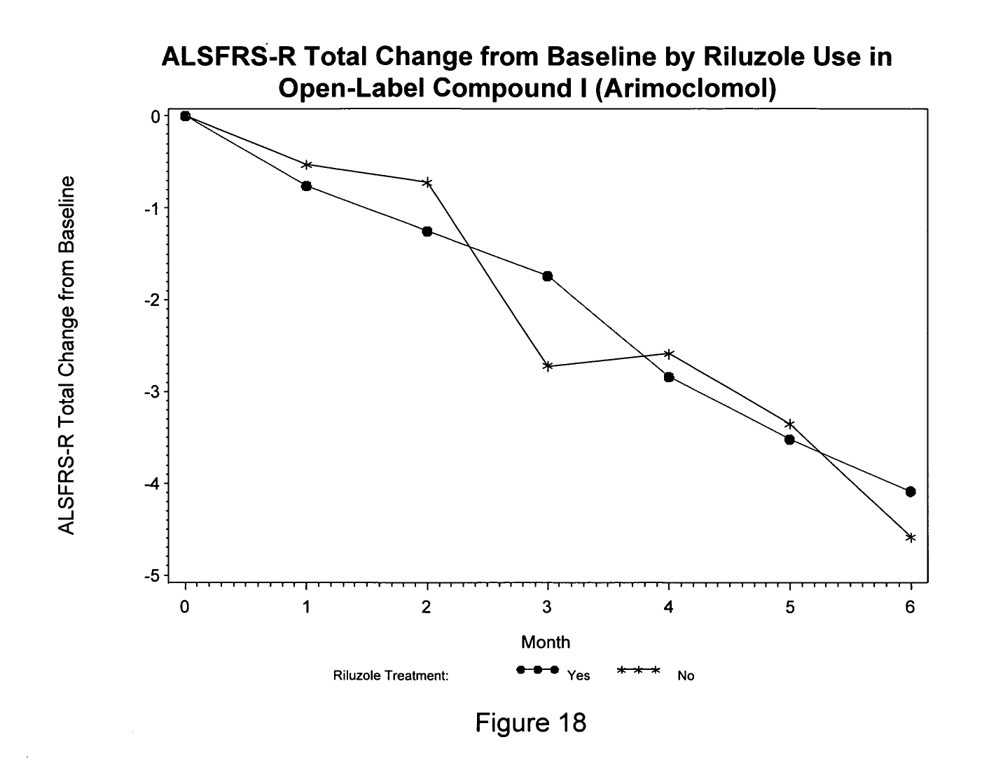

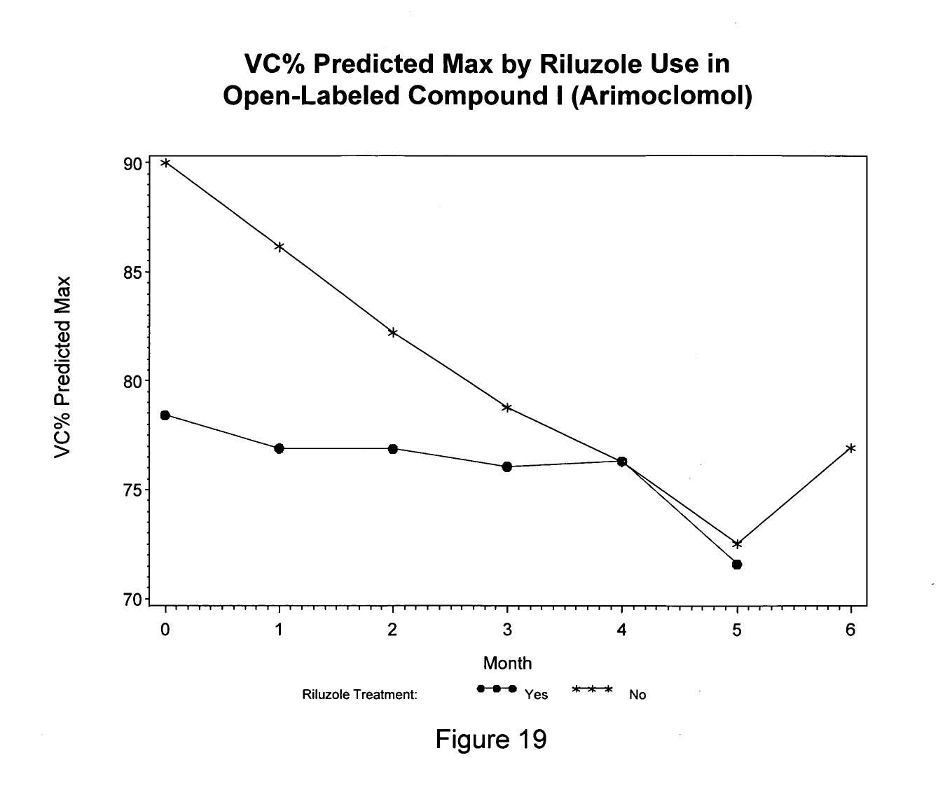

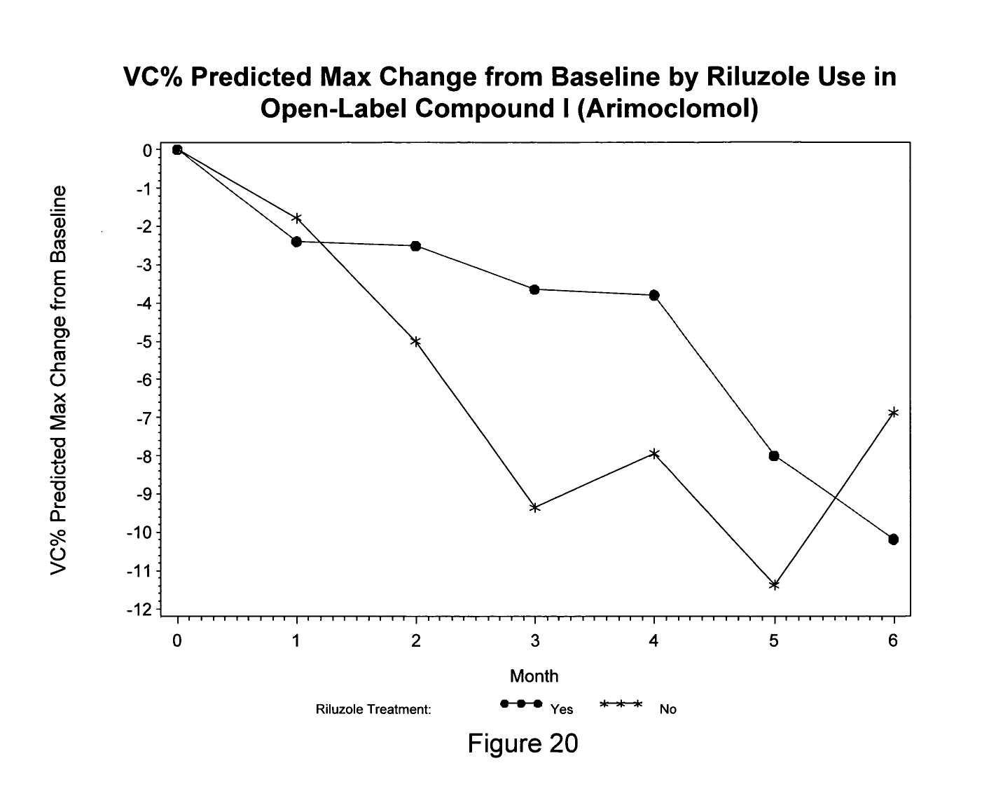

The data was also analyzed with regard to whether or not the patients were treated with 50 mg q.d. riluzole. Unlike the placebo-controlled study as disclosed in example 23, there is no apparent difference in the rate of ALSFRS-R decline in patients co-administered with riluzole. (See FIGS. 17 and 18). However, in this study those patients who were treated with riluzole in addition to Compound I appeared to progress more slowly with regard to theoretical Vital Capacity (VC) versus those not treated with riluzole. (See FIGS. 19 and 20). Previous studies have failed to demonstrate any effect by riluzole alone on VC. Therefore, these data support the conclusion that the combination of riluzole with Compound I may slow disease progression more effectively than treatment with either compound alone.

We claim:

1. A method of treating a condition, disorder or disease in a patient comprising the step of administering to a patient: (a) a pharmaceutical composition comprising a therapeutically effective amount of compound (I): 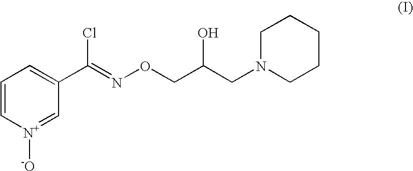
or a pharmaceutically acceptable salt thereof; (b) an additional therapeutic agent; and (c) a pharmaceutically acceptable carrier; wherein the condition, disorder or disease is associated with neurodegeneration in the central nervous system.

2. The method according to claim 1, wherein the condition, disorder or disease is selected from ALS, PD, AD, Huntington's Disease, stroke and cystic fibrosis.

3. The method according to claim 2, wherein Compound I and the additional therapeutic agent are combined into a single dosage form.

4. The method according to claim 2 or 3, wherein the additional therapeutic agent is selected from the group consisting of cholinesterase inhibitors, acetylcholinesterase inhibitors, nerve impulse inhibitors, antioxidants, nonsteroidal anti-inflammatory agents; NMDA antagonists, dopamine agonists, COMT inhibitors, anti-cholinergics, anti-psychotics, anxiolytic agents, dopamine metabolism inhibitors, neuroprotectants, neurotransmitters, neurotransmitter agonists, sedatives, anti-depression agents, neurotransmitter antagonists, stimulants, tranquilizers, and GABA agonists.

5. The method according to claim 2 or 3, wherein the additional therapeutic agent comprises lumilysergol, benzothialzole, riluzole, phenyl benzothialzole, lifarizine or α-tocopherol.

6. The method according to 5, wherein the additional therapeutic agent comprises riluzole.

7. A method of treating ALS in a patient in need thereof comprising the step of administering to a patient a pharmaceutical composition comprising: (a) a compound (I): 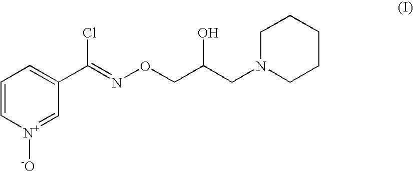
or a pharmaceutically acceptable salt thereof, (b) riluzole; and (c) a pharmaceutically acceptable carrier.

8. A pharmaceutical composition comprising:
(a) a compound represented by formula (III) or its tautomer represented by formula (IV): 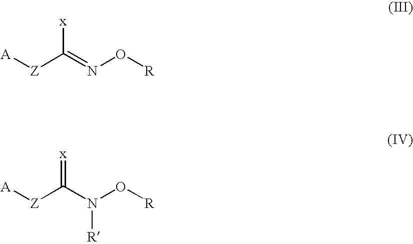
and pharmaceutically acceptable salts thereof, wherein:
A is an alkyl, substituted alkyl, aralkyl, aralkyl substituted in the aryl and/or in the alkyl moiety, aryl, substituted aryl, heteroaryl or substituted heteroaryl group;
Z is a covalent bond, oxygen or ═NR³;
R³ is selected from the group consisting of hydrogen, an alkyl, substituted alkyl, aryl, substituted aryl, aralkyl, or aralkyl substituted in the aryl and/or in the alkyl moiety;
R is an alkyl or substituted alkyl,
X, in compound of formula (III), is halogen or a substituted hydroxy or amino, monosubstituted amino or disubstituted amino group and, in compound of formula (IV), is oxygen, imino or substituted imino group; and
R′ is hydrogen, an alkyl, substituted alkyl, aryl, substituted aryl, aralkyl, aralkyl having substituted aryl and/or alkyl moiety, acyl or substituted acyl group;
(b) an additional therapeutic agent; and
(c) a pharmaceutically acceptable carrier.

9. The pharmaceutical composition according to claim 8, wherein the additional therapeutic agent is selected from an agent to treat ALS, PD, stroke, AD, Huntington's Disease or cystic fibrosis.

10. The pharmaceutical composition according to claim 8, wherein the additional therapeutic agent is selected from cholinesterase inhibitors, acetylcholinesterase inhibitors, nerve impulse inhibitors, antioxidants, nonsteroidal anti-inflammatory agents; NMDA antagonists, dopamine agonists, COMT inhibitors, anti-cholinergics, anti-psychotics, anxiolytic agents, dopamine metabolism inhibitors, neuroprotectants, neurotransmitters, neurotransmitter agonists, sedatives, anti-depression agents, neurotransmitter antagonists, stimulants, tranquilizers, and GABA agonists.

11. The pharmaceutical composition according to claim 8 or 9, wherein the additional therapeutic agent comprises lumilysergol, benzothialzole, riluzole, phenyl benzothialzole, lifarizine or α-tocopherol.

12. The pharmaceutical composition according to claim 11, wherein the additional therapeutic agent comprises riluzole.

13. A method of treating a condition, disorder or disease in a patient comprising the step of administering to a patient a pharmaceutical composition according to any one of claims 8-12; wherein the condition, disorder or disease is associated with neurodegeneration in the central nervous system.

14. The method according to claim 13, wherein the disease is selected from ALS, PD, AD, Huntington's Disease, stroke and cystic fibrosis.

15. A method of treating a condition, disorder or disease in a patient comprising the step of administering to a patient a pharmaceutical composition comprising a therapeutically effective amount of compound (II): 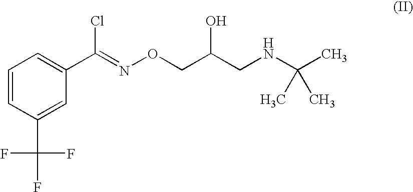
or a pharmaceutically acceptable salt thereof and a pharmaceutically acceptable carrier, wherein the condition, disorder or disease is associated with neurodegeneration in the central nervous system.

16. The method according to claim 15, wherein the condition, disorder or disease is selected from ALS, PD, AD, Huntington's Disease, stroke and cystic fibrosis.

17. The method according to claim 15 or 16, further comprising the step of administering an additional therapeutic agent.

18. The method according to claim 17, wherein Compound II and the additional therapeutic agent are combined into a single dosage form.

19. The method according to claim 17 or 18, wherein the additional therapeutic agent is selected from an agent to treat ALS, PD, stroke, AD, Huntington's Disease or cystic fibrosis.

20. The method according to claim 17 or 18, wherein the additional therapeutic agent is selected from cholinesterase inhibitors, acetylcholinesterase inhibitors, nerve impulse inhibitors, antioxidants, nonsteroidal anti-inflammatory agents; NMDA antagonists, dopamine agonists, COMT inhibitors, anti-cholinergics, anti-psychotics, anxiolytic agents, dopamine metabolism inhibitors, neuroprotectants, neurotransmitters, neurotransmitter agonists, sedatives, anti-depression agents, neurotransmitter antagonists, stimulants, tranquilizers and GABA agonists.

21. The method according to claim 17 or 18, wherein the additional therapeutic agent is lumilysergol, benzothialzole, riluzole, phenyl benzothialzole, lifarizine or α-tocopherol.

22. The method according to claim 21, wherein the additional therapeutic agent comprises riluzole.
